SEQUENCE LISTING (26 sequences)

SEQ ID NO: 1 - protein, 24 aa, Artificial Sequence
  Description of Artificial Sequence Synthetic peptide
RRWVRRVRRVWRRVVRVVRRWVRR

SEQ ID NO: 2 - protein, 14 aa, Artificial Sequence
  Description of Artificial Sequence Synthetic peptide
IRRRRRRIRRRRRR

SEQ ID NO: 3 - protein, 21 aa, Artificial Sequence
  Description of Artificial Sequence Synthetic peptide
IRRRIRRIRRRIRRIRRRIRR

SEQ ID NO: 4 - protein, 21 aa, Artificial Sequence
  Description of Artificial Sequence Synthetic peptide
IRRIIRRIRRIIRRIRRIIRR

SEQ ID NO: 5 - protein, 21 aa, Artificial Sequence
  Description of Artificial Sequence Synthetic peptide
VWRWVRRVWRWVRRVWRWVRR

SEQ ID NO: 6 - protein, 14 aa, Artificial Sequence
  Description of Artificial Sequence Synthetic peptide
VWRWVRRVWRWVRR

SEQ ID NO: 7 - protein, 14 aa, Artificial Sequence
  Description of Artificial Sequence Synthetic peptide
VVRVVRRVVRVVRR

SEQ ID NO: 8 - protein, 21 aa, Artificial Sequence
  Description of Artificial Sequence Synthetic peptide
VVRVVRVVVRVVRVVVRVVRV

SEQ ID NO: 9 - protein, 11 aa, Artificial Sequence
  Description of Artificial Sequence Synthetic peptide
RSRVVRSWSRV

SEQ ID NO: 10 - protein, 84 aa, Artificial Sequence
  Description of Artificial Sequence Synthetic polypeptide
RFVRRVRRFVRRVRRFVRRVRRFVRRVRRFVRRVRRFVRRVRRFVRRVRRFVRRVRRFVRRVRRFVRRVRRFVRRVRRFVRRVR

SEQ ID NO: 11 - protein, 20 aa, Artificial Sequence
  Description of Artificial Sequence Synthetic peptide
RRTYSRSRRTYSRSRRTYSR

SEQ ID NO: 12 - protein, 28 aa, Artificial Sequence
  Description of Artificial Sequence Synthetic peptide
KVVSSIIEIISSVVKVVSSIIEIISSVV

SEQ ID NO: 13 - protein, 20 aa, Artificial Sequence
  Description of Artificial Sequence Synthetic peptide
KKTHTKTKKTHTKTKKTHTK

SEQ ID NO: 14 - protein, 21 aa, Artificial Sequence
  Description of Artificial Sequence Synthetic peptide
VVRVVRRVVRVVRRVVRVVRR

SEQ ID NO: 15 - protein, 12 aa, Artificial Sequence
  Description of Artificial Sequence Synthetic peptide
RVVRVVRRVVRR

SEQ ID NO: 16 - protein, 12 aa, Artificial Sequence
  Description of Artificial Sequence Synthetic peptide
RVVRVVRRWVRR

SEQ ID NO: 17 - protein, 12 aa, Artificial Sequence
  Description of Artificial Sequence Synthetic peptide
RWWRWWRRWWRR

SEQ ID NO: 18 - protein, 18 aa, Artificial Sequence
  Description of Artificial Sequence Synthetic peptide
WRRWWRRWWRWWRRWWRR

SEQ ID NO: 19 - protein, 24 aa, Artificial Sequence
  Description of Artificial Sequence Synthetic peptide
RRVVRRVRRVVRRVVRVVRRVVRR

SEQ ID NO: 20 - protein, 24 aa, Artificial Sequence
  Description of Artificial Sequence Synthetic peptide
RRWWRRWRRWWRRWWRWWRRWWRR

SEQ ID NO: 21 - protein, 36 aa, Artificial Sequence
  Description of Artificial Sequence Synthetic polypeptide
VRRVVRRVVRVVRRVVRRVRRVVRRVVRVVRRVVRR

SEQ ID NO: 22 - protein, 36 aa, Artificial Sequence
  Description of Artificial Sequence Synthetic polypeptide
VRRVWRRVVRVVRRWVRRVRRVWRRVVRVVRRWVRR

SEQ ID NO: 23 - protein, 42 aa, Artificial Sequence
  Description of Artificial Sequence Synthetic polypeptide
RRVVRRVRRVVRRVVRVVRRVVRRVRRVVRRVVRVVRRVVRR

SEQ ID NO: 24 - protein, 48 aa, Artificial Sequence
  Description of Artificial Sequence Synthetic polypeptide
RVVRVVRRVVRRVRRVVRRVVRVVRRVVRRVRRVVRRVVRVVRRVVRR

SEQ ID NO: 25 - protein, 48 aa, Artificial Sequence
  Description of Artificial Sequence Synthetic polypeptide
RVVRVVRRWVRRVRRVWRRVVRVVRRWVRRVRRVWRRVVRVVRRWRVV

SEQ ID NO: 26 - protein, 6 aa, Artificial Sequence
  Description of Artificial Sequence Synthetic 6xHis tag
HHHHHH

(19) United States
(12) Patent Application Publication
(10) Pub. No.: US 2020/0277334 A1
(43) Pub. Date: Sep. 3, 2020

(54) ENGINEERED ANTIMICROBIAL AMPHIPHILIC PEPTIDES AND METHODS OF USE

(71) Applicant: PEPTILOGICS, INC., Pittsburgh, PA (US)

(72) Inventor: Jonathan D. STECKBECK, Pittsburgh, PA (US)

(21) Appl. No.: 16/801,782

(22) Filed: Feb. 26, 2020

Related U.S. Application Data
(63) Continuation of application No. 16/490,164, filed on Aug. 30, 2019.
(60) Provisional application No. 62/466,808, filed on Mar. 3, 2017.

(51) Int. Cl. C07K 14/00; A61P 31/18; A61P 31/04; A01N 33/08; A01N 47/44; A61K 31/145; A61K 38/16; A61K 45/06; A61L 27/28; A61L 27/54; A61L 31/08; A61L 31/16; C07K 1/04

(52) U.S. Cl. CPC C07K 14/001; A61L 2420/06; A61P 31/04; A01N 33/08; A01N 47/44; A61K 31/145; A61K 38/16; A61K 45/06; A61L 27/28; A61L 27/54; A61L 31/08; A61L 31/16; C07K 1/04; A61L 2300/404; A61L 2300/606; A61P 31/18

(57) ABSTRACT

Disclosed herein are novel peptides that can comprise antimicrobial, antiviral, antifungal or antitumor activity when administered to a subject.

### CROSS-REFERENCE

This application is a continuation of U.S. application Ser. No. 16/490,164, filed Aug. 30, 2019, which is the National Stage Entry of International Application No. PCT/US18/20708, filed Mar. 2, 2018, which claims the benefit of U.S. Provisional Application No. 62/466,808, filed on Mar. 3, 2017, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 24, 2020, is named 48615-701_301_SL.txt and is 9,850 bytes in size.

### SUMMARY

Disclosed herein are peptides. Peptides disclosed herein can comprise a polypeptide sequence of Formula A, Formula B, Formula C, Formula D, Formula E, Formula F, Formula G, Formula H, Formula I, Formula J, Formula K, Formula L, Formula M, Formula N, or a salt of any of these; where: Formula A can be (AA₁-AA₂-AA₃-AA₄-AA₅-AA₆-AA₇)ₙ; where AA₁ can be independently X, Ar, or Y; and AA₂, AA₃, AA₄, AA₅, AA₆, and AA₇ can be independently Y, U, $ or @; Formula B can be (AA₁-AA₂-AA₃-AA₄-AA₅-AA₆-AA₇)ₙ; where AA₁ and AA₅ can be independently X, Y, or Ar; and AA₂, AA₃, AA₄, AA₆, and AA₇ can be independently Y, U, $ or @; Formula C can be (AA₁-AA₂-AA₃-AA₄-AA₅-AA₆-AA₇)ₙ; where AA₁ and AA₄ can be independently X, Y, or Ar; and AA₂, AA₃, AA₅, AA₆, and AA₇ can be independently Y, U, $ or @; Formula D can be (AA₁-AA₂-AA₃-AA₄-AA₅-AA₆-AA₇)ₙ; where AA₁ can be independently X, Y, or Ar; AA₄ and AA₅ can be independently X or Ar; AA₂ and AA₇ can be independently U, $ or @; and AA₃ and AA₆ can be independently Y, U, $ or @; Formula E can be (AA₁-AA₂-AA₃-AA₄-AA₅-AA₆-AA₇)ₙ; where AA₁ can be independently X, Y, or Ar; AA₂, AA₄, and AA₅ can be independently X or Ar; and AA₃, AA₆, and AA₇ can be independently Y, U, $ or @; Formula F can be (AA₁-AA₂-AA₃-AA₄-AA₅-AA₆-AA₇)ₙ; where AA₁ can be independently X, Y, or Ar; AA₄, AA₅, and AA₇ can be independently X or Ar; and AA₂, AA₃, and AA₆ can be independently Y, U, $ or @; Formula G can be (AA₁-AA₂-AA₃-AA₄-AA₅-AA₆-AA₇)ₙ; where AA₁, AA₄, AA₅ can be independently X, Y, or Ar; AA₂ and AA₇ can be independently X or Ar; and AA₃ and AA₆ can be independently Y, U, $ or @; Formula H can be (AA₁-AA₂-AA₃-AA₄-AA₅-AA₆AA₇)ₙ; where AA₁ can be independently Y, U, $, or @; AA₃, AA₄, AA₅, and AA₆ can be independently X, Y, or Ar; and AA₂ and AA₇ can be independently X or Ar; Formula I can be (AA₁-AA₂-AA₃-AA₄-AA₅-AA₆AA₇)ₙ; where AA₁ and AA₅ can be independently Y, U, $, or @; AA₃, AA₄, and AA₆ can be independently X, Y, or Ar; and AA₂ and AA₇ can be independently X or Ar; Formula J can be (AA₁-AA₂-AA₃-AA₄-AA₅-AA₆AA₇)ₙ; where AA₁ and AA₄ can be independently Y, U, $, or @; AA₃, AA₅, and AA₆ can be independently X, Y, or Ar; and AA₂ and AA₇ can be independently X or Ar; Formula K can be (AA₁_AA₂-AA₃-AA₄-AA₅-AA₆-AA₇)ₙ; where AA₁, AA₄, and AA₅ can be independently Y, U, $, or @; and AA₂, AA₃, AA₆, and AA₇ can be independently X, Y, or Ar; Formula L can be (AA₁-AA₂-AA₃-AA₄-AA₅-AA₆AA₇)ₙ; where AA₁, AA₂, AA₄, and AA₅ can be independently Y, U, $, or @; and AA₃, AA₆, and AA₇ can be independently X, Y, or Ar; Formula M can be (AA₁-AA₂-AA₃-AA₄-AA₅-AA₆AA₇)ₙ; where AA₁, AA₄, AA₅, and AA₇ can be independently Y, U, $, or @; and AA₂, AA₃, and AA₆ can be independently X, Y, or Ar; and Formula N can be (AA₁-AA₂-AA₃-AA₄-AA₅-AA₆AA₇)ₙ; where AA₁, AA₂, AA₄, AA₅, and AA₇ can be independently Y, U, $, or @; and AA₃ and AA₆ can be independently X, Y, or Ar; where: X can be independently Gly, or an amino acid comprising a C₁-C₁₀ alkyl, C₁-C₁₀ alkenyl, C₁-C₁₀ alkynyl, cycloalkyl, or alkylcycloalkyl side chain; Ar can be an amino acid comprising an aromatic side chain; Y can be an amino acid comprising a side chain that can be at least partially protonated at a pH of about 7.3; U can be an amino acid comprising an amide containing side chain; $ can be an amino acid comprising an alcohol or thiol containing side chain; @ can be an amino acid comprising a side chain that can be at least partially deprotonated at a pH of about 7.3; n can be a number ranging from about 1 to about 7; where at least one AA₁ can be an N-terminal amino acid, where the amino group of the N-terminal amino acid can comprise substituents R′ and R″, where: R′ and R″ can be independently H; phosphoryl; alkyl; alkenyl; alkynyl; cycloalkyl; sulfonyl; sulfinyl; silyl; pyroglutamyl; an alkyl carbonyl which can be substituted with a halogen, an alkyl group, a cylcloalkyl group, or any combination thereof; a thioester, acetyl, a urea, a carbamate, a sulfonamide, an alkylamine, aryl, alkylaryl, a heteroaryl, alkyheteroaryl; or RC(O)—; where R can be independently H, D, alkyl, cycloalkyl, aryl, heteroaryl, alkylaryl, heteroaryl, or alkyheteroaryl; or R′ and R″ together with the nitrogen atom to which they can be attached, form a substituted or non-substituted 5, 6, or 7-membered ring; where the peptide may not comprise 3 or more contiguous arginine or lysine residues; where the peptide may not be a cyclic peptide; and where at least one of the following applies: (i) the peptide, a metabolite thereof, or salt thereof can exhibit antimicrobial activity against a bacteria with a minimum inhibitory concentration ranging from about 0.1 μg/mL to about 100 μg/mL in vitro; (ii) the peptide, a metabolite thereof, or salt thereof can exhibit antiviral activity against a virus with a minimum inhibitory concentration ranging from about 0.1 μg/mL to about 100 μg/mL in vitro; (iii) the peptide or salt thereof can exhibit antifungal activity against a fungus with a minimum inhibitory concentration ranging from about 0.1 μg/mL to about 100 μg/mL in vitro; or (iv) the peptide, a metabolite thereof, or salt thereof can exhibit antitumor activity against a tumor cell with an LD₅₀ of from about 0.01 μM to about 100 μM in vitro. In some embodiments, the peptide or salt thereof can be from about 8 to about 48 amino acids in length. In some embodiments, the peptide or salt thereof can comprise at least one amino acid that can be in a D-configuration. In some embodiments, the peptide or salt thereof may not comprise an amino acid that can be in a D-configuration. In some embodiments, the peptide or salt thereof can comprise at least one amino acid that can be in an L-configuration. In some embodiments, the peptide or salt thereof may not comprise an amino acid that can be in an L-configuration. In some embodiments, the peptide or salt thereof can comprise at least 1 amino acid that may not be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, the peptide or salt thereof can comprise at least 1 unnatural amino acid. In some embodiments, the unnatural amino acid can be a Nuclear Magnetic Resonance (NMR) promoting agent, where the NMR promoting agent can be selected from the group consisting of a spin-labeled compound, a paramagnetic metal chelating compound, a compound comprising an NMR active isotope, and any combination thereof. In some embodiments, the spin-labeled compound can be a nitroxide compound. In some embodiments, the paramagnetic metal chelating compound can be a bipyridine comprising moiety. In some embodiments, the paramagnetic metal chelating compound can be a hydroxyquinoline comprising moiety. In some embodiments, the NMR active isotope can be ¹⁵N. In some embodiments, the NMR active isotope can be ¹³C. In some embodiments, the NMR active isotope can be ³¹P. In some embodiments, the unnatural amino acid can be a fluorescent amino acid. In some embodiments, a peptide can comprise a polypeptide sequence of formula [Y-Ar-X-Y-Y-X-X]ₙ. In some embodiments, a peptide can comprise a polypeptide sequence of formula [U-Ar-X-Y-Y-X-Ar]ₙ. In some embodiments, a peptide can comprise a polypeptide sequence of formula [Y-X-X-$-$-X-X]ₙ. In some embodiments, a peptide can comprise a sequence of formula [Y-X-X-$-$-X-X-@-X-X-$-$-X-X]ₙ where n can be from about 0.5 to about 3.5.

Also disclosed herein are peptides or salts thereof comprising a polypeptide of sequence: Y-X-X-Y-X-X-Y-Y-X-X-Y-Y; Y-X-X-Y-X-X-Y-Y-Ar-X-Y-Y; Y-Ar-Ar-Y-Ar-Ar-Y-Y-Ar-Ar-Y-Y; Ar-Y-Y-Ar-Ar-Y-Y-Ar-Ar-Y-Ar-Ar-Y-Y-Ar-Ar-Y-Y; Y-Y-X-X-Y-Y-X-Y-Y-X-X-Y-Y-X-X-Y-X-X-Y-Y-X-X-Y-Y; Y-Y-Ar-X-Y-Y-X-Y-Y-X-Ar-Y-Y-X-X-Y-X-X-Y-Y-Ar-X-Y-Y; Y-Y-Ar-Ar-Y-Y-Ar-Y-Y-Ar-Ar-Y-Y-Ar-Ar-Y-Ar-Ar-Y-Y-Ar-Ar-Y-Y; X-Y-Y-X-X-Y-Y-X-X-Y-X-X-Y-Y-X-X-Y-Y-X-Y-Y-X-X-Y-Y-X-X-Y-X-X-Y-Y-X-X-Y-Y; X-Y-Y-X-Ar-Y-Y-X-X-Y-X-X-Y-Y-Ar-X-Y-Y-X-Y-Y-X-Ar-Y-Y-X-X-Y-X-X-Y-Y-Ar-X-Y-Y; Y-Y-X-X-Y-Y-X-Y-Y-X-X-Y-Y-X-X-Y-X-X-Y-Y-X-X-Y-Y-X-Y-Y-X-X-Y-Y-X-X-Y-X-X-Y-Y-X-X-Y-Y; Y-X-X-Y-X-X-Y-Y-X-X-Y-Y-X-Y-Y-X-X-Y-Y-X-X-Y-X-X-Y-Y-X-X-Y-Y-X-Y-Y-X-X-Y-Y-X-X-Y-X-X-Y-Y-X-X-Y-Y; or Y-X-X-Y-X-X-Y-Y-Ar-X-Y-Y-X-Y-Y-X-Ar-Y-Y-X-X-Y-X-X-Y-Y-Ar-X-Y-Y-X-Y-Y-X-Ar-Y-Y-X-X-Y-X-X-Y-Y-Ar-Y-X-X; where: X can be independently Gly, or an amino acid comprising a C₁-C₁₀ alkyl, C₁-C₁₀ alkenyl, C₁-C₁₀ alkynyl, cycloalkyl, or alkylcycloalkyl side chain; Ar can be an amino acid comprising an aromatic side chain; and Y can be an amino acid comprising a side chain that can be at least partially protonated at a pH of about 7.3; where the peptide or salt thereof contains at least one amino acid that may not be Val, Trp or Arg; and where the peptide may not be a cyclic peptide.

Also disclosed herein are peptides or salts thereof that can have from about 70% to about 91% homology to sequence selected from the group consisting of: Arg-Val-Val-Arg-Val-Val-Arg Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg- Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); and Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25); where the peptide may not comprise 3 or more contiguous arginine or lysine residues; and where the peptide may not be a cyclic peptide. In some embodiments, the peptide or salt thereof can have a minimum inhibitory concentration against at least one of Staphylococcus aureus, methicillin-resistant Staphylococcus aureus, carbapenem-resistant Staphylococcus aureus, colistin-resistant Staphylococcus aureus, vancomycin-resistant Staphylococcus aureus, Streptococcus pneumonia, methicillin-resistant Streptococcus pneumonia, carbapenem-resistant Streptococcus pneumonia, colistin-resistant Streptococcus pneumonia, vancomycin-resistant Streptococcus pneumonia, carbapenem-resistant Enteroacteriaceae, vancomycin-resistant Enteroacteriaceae, Staphylococcus epidermidis, methicillin-resistant Staphylococcus epidermidis, carbapenem-resistant Staphylococcus epidermidis, colistin-resistant Staphylococcus epidermidis, vancomycin-resistant Staphylococcus epidermidis, Staphylococcus salivarius, methicillin-resistant Staphylococcus salivarius, carbapenem-resistant Staphylococcus salivarius, colistin-resistant Staphylococcus salivarius, vancomycin-resistant Staphylococcus salivarius, Corynebacterium minutissium, methicillin-resistant Corynebacterium minutissium, carbapenem-resistant Corynebacterium minutissium, colistin-resistant Corynebacterium minutissium, vancomycin-resistant Corynebacterium minutissium, Corynebacterium pseudodiphtheriae, methicillin-resistant Corynebacterium pseudodiphtheriae, carbapenem-resistant Corynebacterium pseudodiphtheriae, colistin-resistant Corynebacterium pseudodiphtheriae, vancomycin-resistant Corynebacterium pseudodiphtheriae, Corynebacterium stratium, methicillin-resistant Corynebacterium stratium, carbapenem-resistant Corynebacterium stratium, colistin-resistant Corynebacterium stratium, vancomycin-resistant Corynebacterium stratium, Corynebacterium group G1, Corynebacterium group G2, Streptococcus pneumonia, methicillin-resistant Streptococcus pneumonia, carbapenem-resistant Streptococcus pneumonia, colistin-resistant Streptococcus pneumonia, vancomycin-resistant Streptococcus pneumonia, Streptococcus mitis, methicillin-resistant Streptococcus mitis, carbapenem-resistant Streptococcus mitis, colistin-resistant Streptococcus mitis, vancomycin-resistant Streptococcus mitis, Streptococcus sanguis, methicillin-resistant Streptococcus sanguis, carbapenem-resistant Streptococcus sanguis, colistin-resistant Streptococcus sanguis, vancomycin-resistant Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni ranging from about 0.1 μg/mL to about 100 μg/mL. In some embodiments, the peptide or salt thereof can have a minimum inhibitory concentration against methicillin resistant Staphylococcus aureus ranging from about 0.1 μg/mL to about 100 μg/mL. In some embodiments, the antimicrobial activity can be against a bacteria that can be resistant to an antibiotic selected from the group consisting of a cephalosporin, a fluoroquinolone, a carbapenem, a colistin, an aminoglycoside, vancomycin, streptomycin, and methicillin. In some embodiments, the peptide or salt thereof at least partially adopts an α-helical structure when contacted with a bacterial membrane, a viral envelope, or a tumor cell membrane as measured by circular dichroism or NMR spectroscopy. In some embodiments, at least a portion of the α-helical structure can be amphipathic. In some embodiments, the peptide or salt thereof can be at least partially conformationally constrained. In some embodiments, the peptide or salt thereof can be at least partially constrained as an alpha helix. In some embodiments, the peptide or salt thereof can be constrained at least in part with a disulfide bond, a staple, a stitch, or any combination thereof. In some embodiments, when the peptide or salt thereof can be administered to a primate, the peptide or salt thereof can be substantially localized in a liver, a spleen, or a kidney of the primate. In some embodiments, the peptide or salt thereof can be recombinant. In some embodiments, the peptide or salt thereof can be chemically synthesized. In some embodiments, the peptide or salt thereof can be isolated and purified. In some embodiments, the peptide or salt thereof can be in the form of a cleavable prodrug.

Also disclosed herein are pharmaceutical formulations comprising: (a) a peptide or salt thereof disclosed herein; and (b) at least one of: an excipient, a diluent, or a carrier. In some embodiments, the excipient can be a chelator. In some embodiments, the chelator can be a fungicidal chelator. In some embodiments, the diluent can be an aqueous acid. In some embodiments, the pharmaceutical formulation can further comprise cysteamine. In some embodiments, the pharmaceutical formulation can be in unit dose form. In some embodiments, the pharmaceutical formulation can be in the form of a tablet, a liquid, a syrup, an oral formulation, an intravenous formulation, an intranasal formulation, an ocular formulation, an otic formulation, a suppository, and any combination thereof.

Also disclosed herein are pharmaceutical formulations comprising: (a) a peptide or salt thereof comprising from about 70% to about 100% homology to a polypeptide of sequence: Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); or Arg-Val-Val-Arg-Val-V al-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-V al-Arg-Val-Val-Arg-Arg-Trp-Arg-Val-Val (SEQ ID NO: 25); and (b) at least one of: an excipient, a diluent, or a carrier; where the formulation can be in unit dose form, where the peptide may not comprise 3 or more contiguous arginine or lysine residues; where the peptide may not be a cyclic peptide; and where at least one of the following applies: (i) the peptide or salt thereof can exhibit antimicrobial activity against a bacteria with a minimum inhibitory concentration ranging from about 0.1 μg/mL to about 100 μg/mL in vitro; (ii) the peptide or salt thereof can exhibit antifungal activity against a fungus with a minimum inhibitory concentration ranging from about 0.1 μg/mL to about 100 μg/mL in vitro; (iii) the peptide or salt thereof can exhibit antiviral activity against a virus with a minimum inhibitory concentration ranging from about 0.1 μg/mL to about 100 μg/mL in vitro; or (iv) the peptide or salt thereof can exhibit antitumor activity against a tumor cell with an LD₅₀ of from about 0.01 μM to about 100 μM in vitro. In some embodiments, the excipient can be a chelator. In some embodiments, the chelator can be a fungicidal chelator. In some embodiments, the diluent can be an aqueous acid. In some embodiments, the pharmaceutical formulation can further comprise cysteamine. In some embodiments, a pharmaceutical formulation can further comprise a surfactant. In some embodiments, a surfactant can be selected from the group consisting of a polyoxyethylene sorbitan fatty acid ester, sodium lauryl sulphate, sodium stearyl fumarate, a polyoxyethylene alkyl ether, a sorbitan fatty acid ester, polyethylene glycols, a polyoxyethylene castor oil derivative, docusate sodium, a quaternary ammonium compound, a sugar ester of a fatty acid, a glyceride of a fatty acid, and any combination thereof. In some embodiments, a pharmaceutical formulation can further comprise a small molecule selected from the group consisting of imidazole, indole, nitric oxide, a triazole, a phenol, a sulfide, a polysaccharide, a furanone, a bromopyrrole, and any combination thereof. In some embodiments, the pharmaceutical formulation can be in the form of a tablet, a liquid, a syrup, an oral formulation, an intravenous formulation, an intranasal formulation, an ocular formulation, an otic formulation, a suppository, and any combination thereof. In some embodiments, at least about 80% by weight of the peptide or salt thereof can be present at the end of a 2 year period, as determined by (a) loading a sample of the peptide or salt thereof on an high performance liquid chromatography (HPLC) equipped with a size exclusion column that can be at least about 6 inches in length and can comprise a silica gel; and (b) performing mass spectroscopy on at least one sample eluted from the size exclusion column; where said pharmaceutical formulation can be stored in a closed container at 25° C. at 50% atmospheric relative humidity. In some embodiments, when the peptide, the salt thereof, or the pharmaceutical formulation can be administered to a primate, the peptide or salt thereof has a Tₘₐₓ of from about 1 minute to about 1 hour, a Cₘₐₓ of at least about 100 ng/mL, an AUC_{0>24 hour} of from about 0.1 μg·hr/L to about 1,000 μg·hr/L, or a combination thereof. In some embodiments, when the pharmaceutical formulation can be administered to a primate, the peptide or salt thereof can be substantially localized in a liver, a spleen, or a kidney of the primate. In some embodiments, when the pharmaceutical formulation can be administered to a primate, the peptide or salt thereof has a half-life that can be from about 2 hours to about 24 hours.

Also disclosed herein are methods of inactivating an enveloped virus comprising contacting the enveloped virus with a peptide, salt thereof or pharmaceutical formulation disclosed herein.

Also disclosed herein are methods of inhibiting the growth of or killing a bacterium comprising contacting the bacterium with a peptide, salt thereof or pharmaceutical formulation described herein.

Also disclosed herein are methods that can comprise contacting a bacterium with a composition. In some cases, a composition can comprise a peptide or salt thereof as described herein. In some cases, a composition can comprise a pharmaceutical formulation as described herein. In some cases, a composition can comprise an additional agent. In some cases, an additional agent can at least partially inhibit a formation of, or destroys, a biofilm produced by a bacterium. In some cases, an additional agent can be a surfactant. In some cases, a surfactant can be selected from the group consisting of a polyoxyethylene sorbitan fatty acid ester, sodium lauryl sulphate, sodium stearyl fumarate, a polyoxyethylene alkyl ether, a sorbitan fatty acid ester, polyethylene glycols, a polyoxyethylene castor oil derivative, docusate sodium, a quaternary ammonium compound, a sugar ester of a fatty acid, a glyceride of a fatty acid, and any combination thereof. In some cases, an additional agent can be a small molecule. In some cases, a small molecule can be selected from the group consisting of imidazole, indole, nitric oxide, a triazole, a phenol, a sulfide, a polysaccharide, a furanone, a bromopyrrole, and any combination thereof. In some cases, an additional agent is an amino acid or a derivative thereof. In some cases, an amino acid or derivative thereof can comprise L-leucine or cysteamine.

Also disclosed herein are methods of inhibiting the growth of or killing a tumor cell comprising contacting the tumor cell with a peptide, salt thereof, or pharmaceutical formulation described herein.

Also disclosed herein are methods of treating a bacterial infection comprising administering to a primate a therapeutically effective amount of a peptide, salt thereof, or pharmaceutical formulation described herein for a treatment duration. In some embodiments, the administration of the peptide, salt thereof, or pharmaceutical formulation at least partially ameliorates the bacterial infection after administration to the primate. In some embodiments, prior to the administering, at least one of the following applies: (a) the primate has been previously diagnosed as having the bacterial infection, or (b) the primate can be diagnosed with the bacterial infection. In some embodiments, the bacteria can be selected from the group consisting of an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, and any combination thereof. In some embodiments, the bacteria can secrete a biofim, can be at least partially contained in a biofilm, or a combination thereof. In some embodiments, the administration can be intra-arterial, intravenous, intramuscular, oral, subcutaneous, intranasal, inhalable, or any combination thereof. In some embodiments, the method can further comprise administering an additional antibiotic or an antiviral compound. In some embodiments, the additional antibiotic can be selected from the group consisting of Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof. In some embodiments, the antiviral compound can be selected from the group consisting of Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, a salt of any of these, and any combination thereof. In some embodiments, the treatment duration can be from about 5 days to about 30 days. In some embodiments, the administration can be performed at least once a day. In some embodiments, the administration can be performed at least twice a day. In some embodiments, the primate can be in need thereof. In some embodiments, the primate can be a human. In some embodiments, the human can be a child. In some embodiments, the human can be an adult. In some embodiments, the human can be age 0-18 years old. In some embodiments, the human can be age 18-130 years old. In some embodiments, the human can be a male. In some embodiments, the human can be a female.

Also disclosed herein are methods of treating a viral infection comprising administering to a primate a peptide, salt thereof, or pharmaceutical formulation described herein for a treatment duration. In some embodiments, the administration of the peptide, salt thereof, or pharmaceutical formulation at least partially ameliorates the viral infection after administration to the primate. In some embodiments, prior to the administering, at least one of the following applies: (a) the primate has been previously diagnosed as having the viral infection, or (b) the primate can be diagnosed with the viral infection. In some embodiments, the virus can be an enveloped virus. In some embodiments, the enveloped virus can be selected from the group consisting of a herpesvirus, a poxvirus, a hepadnavirus, a flavivirus, a togavirus, a coronavirus, hepatitis C, hepatitis D, an orthomyxovirus, a paramyxovirus, a rhabdovirus, a bunyavirus, a Filovirus, an alphavirus, an arenavirus, a lentivirus, and any combination thereof. In some embodiments, the administration can be intra-arterial, intravenous, intramuscular, oral, subcutaneous, intranasal, inhalable, or any combination thereof. In some embodiments, the method can further comprise administering an antibiotic or an additional antiviral compound. In some embodiments, the antibiotic can be selected from the group consisting of Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof. In some embodiments, the additional antiviral compound can be selected from the group consisting of Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, a salt of any of these, and any combination thereof. In some embodiments, the treatment duration can be from about 5 days to about 30 days. In some embodiments, the administration can be performed at least once a day. In some embodiments, the administration can be performed at least twice a day. In some embodiments, the primate can be in need thereof. In some embodiments, the primate can be a human. In some embodiments, the human can be a child. In some embodiments, the human can be an adult. In some embodiments, the human can be age 0-18 years old. In some embodiments, the human can be age 18-130 years old. In some embodiments, the human can be a male. In some embodiments, the human can be a female.

Also disclosed herein are methods of treating a cancer comprising administering to a primate a peptide, salt thereof, or pharmaceutical formulation described herein for a treatment duration. In some embodiments, the administration of the peptide, salt thereof, or pharmaceutical formulation at least partially inhibits the growth of a tumor after administration to the primate. In some embodiments, prior to the administering, at least one of the following applies: (a) the primate has been previously diagnosed as having the cancer, or (b) the primate can be diagnosed with the cancer. In some embodiments, the administration can be intra-arterial, intravenous, intramuscular, oral, or any combination thereof. In some embodiments, the cancer can be selected from the group consisting of: leukemia; melanoma; squamous cell carcinoma; neuroblastoma; colorectal adenocarcinoma; lymphoma; prostate; renal; glioblastoma; rhabdomyosarcoma; breast cancer; metastatic breast cancer; and astrocytoma. In some embodiments, the method can further comprise administering an additional anticancer compound or a salt thereof. In some embodiments, the additional anticancer compound can be selected from the group consisting selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof. In some embodiments, the treatment duration can be from about 5 days to about 30 days. In some embodiments, the administration can be performed at least once a day. In some embodiments, the administration can be performed at least twice a day. In some embodiments, the primate can be in need thereof. In some embodiments, the primate can be a human. In some embodiments, the human can be a child. In some embodiments, the human can be an adult. In some embodiments, the human can be age 0-18 years old. In some embodiments, the human can be age 18-130 years old. In some embodiments, the human can be a male. In some embodiments, the human can be a female.

Also disclosed herein are methods of administering a peptide or salt thereof to a subject, where the administration results in a PK profile substantially as depicted in FIG. 4 after an intravenous administration of the peptide or salt thereof at a dose of about 1 mg/kg, about 5 mg/kg, or about 10 mg/kg relative to a body weight of the subject. In some embodiments, the subject can be a rat.

Also disclosed herein are biological coatings comprising a peptide, salt thereof, or pharmaceutical formulation described herein. In some embodiments, the biological coating can be in the form of a film.

Also disclosed herein are methods of making a biological coating comprising contacting a coating material with a peptide, salt thereof, or pharmaceutical formulation described herein. In some embodiments, the coating material can be a film.

Also disclosed herein are compositions comprising: (a) an article and (b) a peptide, salt thereof, or pharmaceutical formulation described herein. In some embodiments, the article can be a medical device. In some embodiments, the medical device can be an implantable medical device. In some embodiments, the implantable device can be a prosthetic limb.

Also disclosed herein are methods of making a pharmaceutical formulation comprising contacting a peptide or salt thereof described herein with at least one of: an excipient, a diluent, or a carrier. In some embodiments, the excipient can be a fungicidal chelator. In some embodiments, the diluent can be an aqueous acid.

Also disclosed herein are kits comprising a peptide, salt thereof, or pharmaceutical formulation described herein and a container. In some embodiments, the kit can comprise instructions for use.

Also disclosed herein are methods of making a kit comprising combining a peptide, salt thereof, or pharmaceutical formulation described herein with a container. In some embodiments, the method can further comprise an addition of instructions for use.

Also disclosed here are methods of making a peptide or salt thereof comprising synthesizing the peptide or salt thereof on a solid support.

Also disclosed herein are methods of making a peptide or salt comprising synthesizing the peptide or salt thereof in a microorganism. In some embodiments, a peptide or salt thereof can be recombinantly produced.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIGS. 1A-1C depict exemplary design considerations for rational design of a peptide described herein. FIG. 1A depicts optimization of amphipathicity. FIG. 1B depicts optimization of peptide length. FIG. 1C depicts optimization of charge and polarity distribution.

FIG. 2 depicts a plot of the mean serum concentration of an exemplary peptide after administration to the cohort of male cynomolgous monkeys (Macaca fascicularis).

FIG. 3 depicts a plot of the mean serum concentration of an exemplary peptide after administration to the cohort of male CD-1 mice.

FIG. 4 depicts a plot of the mean serum concentration of an exemplary peptide after administration to the cohort of male Sprague-Dawley rats.

FIG. 5 depicts an exemplary analysis of a sample from a subject in a clinical trial.

FIG. 6 depicts MIC distributions of an exemplary peptide and comparator drugs against the E. faecium isolates.

FIG. 7 depicts MIC distributions of an exemplary peptide and comparator drugs against the S. aureus isolates.

FIG. 8 depicts MIC distributions of an exemplary peptide and comparator drugs against the K. pneumoniae isolates.

FIG. 9 depicts MIC distributions of an exemplary peptide and comparator drugs against the Acinetobacter isolates.

FIG. 10 depicts MIC distributions of an exemplary peptide and comparator drugs against the P. aeruginosa isolates.

FIG. 11 depicts MIC distributions of an exemplary peptide and comparator drugs against the E. aerogenes isolates.

FIG. 12 depicts MIC distributions of an exemplary peptide and comparator drugs against the E. coli isolates.

FIG. 13 depicts the ability of an exemplary peptide to disrupt a biofilm, as determined by a change in the absorbance at 550 nm, for P. aeruginosa.

FIG. 14 depicts the ability of an exemplary peptide to disrupt a biofilm, as determined by a change in the absorbance at 550 nm, for S. aureus.

### DETAILED DESCRIPTION

### I. Overview

Ant-infective peptides are key effector molecules of the innate immune system and integral components of the first line of defense against infections. Disclosed herein are novel peptides and variants thereof that comprise antimicrobial, antiviral, antifungal or antitumor activity when administered to a subject. A peptide described herein can be used to disrupt the integrity of a membrane by (a) binding to a negatively charged surface on a membrane; and/or (b) integrating into a membrane. The ability of a peptide disclosed herein to bind to a negatively charged surface on a membrane and/or integrate into a membrane can allow a peptide to act as a toxic agent to cells with a negatively charged surface by disrupting membrane integrity.

A peptide disclosed herein can be engineered as a novel therapeutic employing and/or designed with the consideration of one or more of the following principles:
(i) ability to adopt an α-helical structure;
(ii) localization of positively-charged moieties;
(iii) optimization of amphipathicity (or amphilicity);
(iv) optimizing the length of the peptide.

Employing at least one of the principles described can be used to rationally design peptides that are able to bind to a negatively charged surface on a membrane and/or integrate into a membrane for use as a therapeutic agent.

In some exemplary embodiments, a peptide disclosed herein can be an α-helical peptide. In the case of an alpha helix, a rational design of a peptide can employ of one or more of the following principles:
(i) ability to adopt the α-helical structure when contacted with a membrane;
(ii) localization of positively-charged moieties on a face of the helix;
(iii) optimization of amphipathicity (or amphilicity) by localizing a varying number of polar and nonpolar residues on opposing faces of the helix;
(iv) alignment of aromatic residues along an axis between the hydrophobic and hydrophilic faces;
(v) burying a positively-charged moiety on the non-polar or hydrophobic face of the helix;
(vi) optimizing the length of the peptide.

Also disclosed herein are compositions that can comprise a peptide described herein. A composition can be formulated for administration to a subject in order to treat a disease or condition. In some cases, a peptide disclosed herein can bind to a negatively charged surface on a membrane and/or integrate into a membrane to produce a therapeutically useful result.

The methods of treating a disease or condition described herein can be by administering to a subject a peptide or composition containing a peptide disclosed therein. For example, a peptide or composition comprising a peptide described herein can be administered as an antimicrobial agent in order to at least partially inhibit the growth of a pathogen such as a bacteria through disruption of the structural integrity of the bacterial cell membrane. A peptide described herein can be screened for broad spectrum activity against a variety of pathogens for broad utility when administered to a subject.

An antimicrobial peptide described herein can also be used as a means to produce an antimicrobial film for coating a device. In some instances, the peptides disclosed herein can be used to coat the interior and/or exterior of a medical device, for example, an implantable medical device. The coating of a device with a peptide disclosed herein can reduce the growth and proliferation of cells, bacteria, fungi or virus on a surface coated with a peptide. In some instances, coating an implantable medical device with a peptide disclosed herein can reduce the risk of an infection to a subject upon implanting the medical device in a subject.

It is further envisaged that a peptide described herein or composition comprising a peptide described herein can be included in a kit. The kit can be utilized, for example, by a subject or healthcare professional to coat a device or to treat a condition or disease described herein.

### II. Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, a peptide that is “substantially localized” in an organ can indicate that about 90% by weight of a peptide, salt, or metabolite is present in an organ relative to a total amount of a peptide, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances a disease or condition can be cancer. A subject can be a patient. A subject can be an individual. In some instances, a subject, patient or individual can be used interchangeably.

The terms “treat,” “treating”, “treatment,” “ameliorate” or “ameliorating” and other grammatical equivalents as used herein, can include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition.

The term “preventing” can mean preventing additional symptoms, ameliorating or preventing the underlying metabolic causes of symptoms, and can include prophylaxis.

In some instances, “treat,” “treating”, “treatment,” “ameliorate” or “ameliorating” and other grammatical equivalents can include prophylaxis. “Treat,” “treating”, “treatment,” “ameliorate” or “ameliorating” and other grammatical equivalents can further include achieving a therapeutic benefit and/or a prophylactic benefit. Therapeutic benefit can mean eradication of the underlying disease being treated. Also, a therapeutic benefit can be achieved with the eradication of one or more of the physiological symptoms associated with the underlying disease such that an improvement can be observed in a subject notwithstanding that, in some embodiments, the subject can still be afflicted with the underlying disease.

The terms “effective amount”, “therapeutically effective amount” or “pharmaceutically effective amount” as used herein, can refer to a sufficient amount of a compound being administered which will at least partially ameliorate a symptom of a disease or condition being treated.

The terms “compound”, “agent”, or “therapeutic agent” can be used to refer to a peptide as described herein. In some cases, the terms “additional compound”, “additional agent”, or “additional therapeutic agent” can be used to refer to a peptide as described herein. In some cases, the terms “additional compound”, “additional agent”, or “additional therapeutic agent” can be used to refer to a compound, agent, or therapeutic that may not be a peptide described herein. For example, an additional agent can include an antioxidant, an antibiotic, an antifungal, an antiviral, an antineoplastic, a neoadjuvant, and the like. In some instances, “compound, “agent”, and “therapeutic agent” can be used interchangeably.

The terms “peptide” and “polypeptide” can be used interchangeably to encompass both naturally-occurring and non-naturally occurring proteins, and fragments, mutants, derivatives and analogs thereof. A polypeptide may be monomeric or polymeric. Further, a polypeptide may comprise a number of different domains each of which has one or more distinct activities. For the avoidance of doubt, a “polypeptide” may be any length greater two amino acids. A peptide can comprise an overall charge based on pka of side chains of component amino acids. In some instances, a peptide can have an overall positive charge. In some instances, a peptide can have an overall negative charge. In some instances, a peptide can have an overall neutral charge. A peptide can furthermore exist as a zwitterion.

A peptide described herein can be useful as an antimicrobial peptide, for example, against bacteria, fungi, yeast, parasites, protozoa and viruses. The term, “antimicrobial peptide” can be used herein to define any peptide that has microbicidal and/or microbistatic activity and encompasses, non-exclusively, any peptide described as having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties.

The term “recombinant” can refer to a biomolecule, e.g., a gene or protein, that (1) can be removed from its naturally occurring environment, (2) can be isolated from all or a portion of a polynucleotide in which the gene may be found in nature, (3) can be operatively linked to a polynucleotide which it may not be linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism can be recombinant, for example, if it is synthesized from an mRNA synthesized from a recombinant gene present in the cell.

The term “homology” can refer to a % identity of a polypeptide to a reference polypeptide. As a practical matter, whether any particular polypeptide can be at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters can be set such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 amino acid residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

The terms “co-administration”, “administered in combination with” and their grammatical equivalents or the like, as used herein, can encompass administration of selected therapeutic agents to a subject, and can include treatment regimens in which agents are administered by the same or different route of administration or at the same or different times. In some embodiments, a peptide disclosed herein can be co-administered with other agents. These terms can encompass administration of two or more agents to a subject so that both agents and/or their metabolites are present in the subject at the same time. They can include simultaneous administration, administration at different times, and/or administration in a composition in which both agents are present. Thus, in some embodiments, a peptide and an additional agent(s) can be administered in a single composition. In some embodiments, a peptide and an additional agent(s) can be admixed in the composition. In some embodiments, a same peptide or agent can be administered via a combination of different routes of administration. In some embodiments, each agent administered can be in a therapeutically effective amount.

As used herein, the term “bioavailability” can denote the degree to which a drug such as a peptide, salt, metabolite, or other substance becomes available to the target tissue after administration.

Parameters often used in pharmacokinetic (PK) studies can include Tmax, Cmax, AUC(0-∞), AUC(0-t), and T_{1/2} and CL/F. “Tmax” can refer to the time to reach the maximal plasma concentration (“Cmax”) after administration of a therapeutic; “AUC(0-∞)” can refer to the area under the plasma concentration versus time curve from time 0 to infinity; “AUC(0-t)” can refer to the area under the plasma concentration versus time curve from time 0 to time t; “T_{1/2}” can refer to a half-life of a therapeutic in blood plasma; “T_{1/2, elim}” can refer to the half-life of elimination of the therapeutic from circulation; and “CL/F” can refer to an apparent clearance rate of a therapeutic.

### III. Peptides

Disclosed herein are novel, rationally designed peptides engineered for use as novel therapeutics. In some instances, a rationally designed peptide can be used as an antimicrobial, antiviral, antifungal, or antitumor agent when administered to a subject. In other embodiments, a peptide disclosed herein can comprise a random design and having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties.

The production of novel antimicrobial agents is paramount due to the emergence of pathogens resistant to traditional antimicrobial compounds. Thus, there is a long felt, unmet need for new and effective antimicrobial agents.

In some instances, a peptide disclosed herein can be rationally designed to mimic a host-derived peptide. The use of host-derived peptides can be advantageous in that host-derived peptides can potentially mitigate adverse host reactions upon administration to a subject. The potential of using host-derived peptides as antimicrobial agents is described in an article by Hancock et al. Such peptides are of interest due to their role in innate vertebrate immunity. In some instances, these host derived peptides can comprise a portion of neutrophil proteins involved in immunity, for example vertebrate immunity. In some instances, peptides can be cationic peptides comprising an overall positive charge on a surface of the peptide. In other instances, peptides can have an overall neutral or negative change on a surface of the peptide. In some instances, a peptide can fit into at least one structural category: (i) β-sheet structures that are stabilized by multiple disulfide bonds (e.g., human defensin-1), (ii) covalently stabilized loop structures (e.g., bactenecin), (iii) tryptophan (Trp)-rich, extended helical peptides (e.g., indolicidin), and (iv) amphipathic α-helices (e.g., the magainins and cecropins).

While host-derived peptides can be potent antimicrobials, host-derived peptides have typically evolved against specific pathogens. Such specificity can limit their use as broad spectrum antimicrobials. On the other hand, novel protein scaffolds can be designed employing a similar structural motif to a host-derived peptide for use as antimicrobial therapeutics.

The use of a protein scaffold based on lentiviral lytic proteins (LLPs) as a model for engineering broad spectrum antimicrobial compounds is described in U.S. Pat. No. 6,887,847. LLP based peptide analogs can be designed utilizing, for example, the following principles: (i) optimizing amphipathicity, (ii) substituting arginine (Arg) on the charged face and/or valine (Val) or tryptophan (Trp) on the hydrophobic face with another amino acid, and (iii) increasing peptide length.

Other peptide scaffolds can be engineered using similar concepts while employing rational design to increase overall potency and pharmacokinetics of administered agents. In some cases, a peptide that is substantially helical can be used. Examples can include a single helix, coiled-coils, 4-helix bundles, globulins, and the like. In some cases, a peptide that is substantially composed of β-strands can be used. Examples can include structures such as β-sheets, SH3 domains, β-hairpins, Greek keys, β-propellers, β-barrels, immunoglobulins, and the like. In some cases, a peptide can be composed of both α-helices and β-strands. Examples can include zinc fingers, TIM barrels, ferredoxins, SH2 domains, leucine-rich-repeat (LRR) proteins, flavodoxins, and the like. In some cases, novel, non-canonical scaffolds such as those described in U.S. Pat. Nos. 6,548,249 and 6,818,418 can be employed.

Design Principles

Disclosed herein are novel peptides for use as antimicrobial, antiviral, antifungal and/or antitumor agents. In some instances, a peptide can be a rationally designed peptide. In some cases, a rationally designed peptide can comprise a linear structure. In some cases, a linear structure can be at least transient. In some cases, a rationally designed peptide can comprise a cyclical structure. In some cases, a cyclical structure can be at least transient. In some cases, a rationally designed peptide can comprise a helical structure. In some cases, a helical structure can be at least transient. A peptide with a structure as described herein can be engineered and/or optimized to increase the potency of a therapeutic. A peptide disclosed herein can be engineered as a novel therapeutic employing and/or designed with the consideration of one or more of the following principles:
(i) ability to adopt an α-helical structure;
(ii) localization of positively-charged moieties;
(iii) optimization of amphipathicity (or amphilicity);
(iv) optimizing the length of the peptide.

Employing at least one of the principles described above can be used to rationally design peptides able to bind to a negatively charged surface on a membrane and/or integrate into a membrane for use as a therapeutic agent.

In some exemplary embodiments, a peptide disclosed herein can be an α-helical peptide. In the case of an alpha helix, a rational design of a peptide can employ of one or more of the following principles:
(i) ability to adopt the α-helical structure when contacted with a membrane;
(ii) localization of positively-charged moieties on a face of the helix;
(iii) optimization of amphipathicity (or amphilicity) by localizing a varying number of polar and nonpolar residues on opposing faces of the helix;
(iv) alignment of aromatic residues along an axis between the hydrophobic and hydrophilic faces;
(v) burying a positively-charged moiety on the non-polar or hydrophobic face of the helix;
(vi) optimizing the length of the peptide.

In some instances, an α-helical peptide described herein can conform to at least one of principles listed herein. In some instances, a peptide described herein can conform to at least 1, 2, 3, 4, 5, or all 6 of principles described herein.

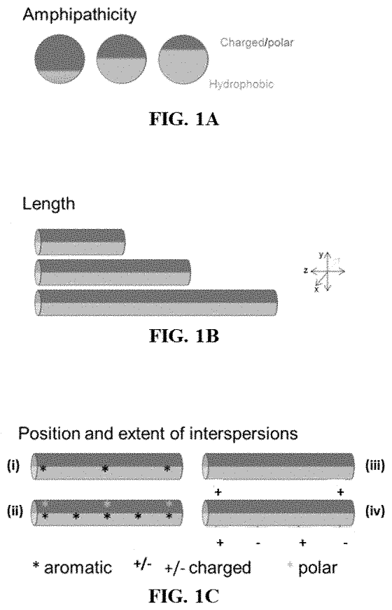

FIGS. 1A-1C depict exemplary design principles employed in the design of a polypeptide described herein.

In some cases, the peptide can be at least partially conformationally constrained. A constrained peptide can be a helical peptide, a cyclic peptide, and the like. Examples of constraining means can include a disulfide bond, a staple, a stich, and the like.

In some cases, a peptide can be engineered to modulate an overall amphipathicity of a peptide. FIG. 1A depicts an exemplary model of an α-helical peptide. In the exemplary model, the distribution of polar and non-polar residues can be arranged along a helical structure such that a distribution of the polar and non-polar residues are adjusted along a face of the helix. The three exemplary models displayed include a helix that can be mostly polar, a helix with approximately equal distribution of polar and non-polar residues on opposite face of the helix, and a helix that can be mostly hydrophobic. A person of skill in the art would be capable of modulating a helix employing this principle to construct a peptide with any such distribution of polar and non-polar residues as desired.

In some instances, amino acid substitutions can be carried out in order to modulate biological activity of a peptide disclosed herein. In some cases, a substitution described herein can be performed to at least maintain biological function of a peptide disclosed herein.

A peptide can be designed to optimize pharmacokinetic parameters. For instance, a peptide can be designed to bear a hydrophobic and/or charged surface to increase associate with a protein, for example, serum albumin. In some embodiments, such an association can increase the resident circulatory half-life of a peptide by allowing a peptide to exceed the renal filtration size cutoff when associated with a protein, for example, serum albumin. In some cases, a peptide, salt, or metabolite thereof can at least partially associate with a protein, cell, polynucleotide or a fragment thereof. In some embodiments, a peptide, salt, or metabolite thereof can be at least partially associate with serum albumin.

In some cases, a peptide can be engineered to modulate an overall length of a polypeptide described herein. FIG. 1B depicts an exemplary model of this principle, in which a peptide length can be adjusted in an α-helical peptide to increase a length of the helix.

In some instances, a peptide can be engineered to incorporate a repeating motif within a peptide. Various motifs and secondary structures have been described herein. FIG. 1C depicts exemplary, non-limiting models of α-helical peptides employing exemplary repeating motifs. FIG. 1C(i) shows a helix in which an aromatic residue can be positioned along an interface between a hydrophobic and hydrophilic face of a helix. FIG. 1C(ii) shows a helix in which a polar residue can be positioned immediately prior to an aromatic residue positioned as described in FIG. 1C(i). The exemplary helix model in FIG. 1C(ii) depicts an alternating motif in which the polar residue can be positioned immediately prior to the aromatic residue every other turn of the helix, though the motif can be adjusted accordingly within the skill of a skilled artisan. FIG. 1C(iii) shows a helix in which a pair of positively-charged amino acids are place on opposite ends of a helix on the hydrophobic face of the helix. FIG. 1C(iv) shows a motif in which positively and negatively charged amino acids are positioned on the hydrophobic face of the helix in an alternating pattern, such that each turn of the helix can comprise a positively or negatively charged amino acid. FIGS. 1A, 1B, and 1C are not meant to be limiting. A person of skill in the art would be capable of employing the principles disclosed herein to construct a peptide having a desired property and function.

In some instances, the length of a peptide can be varied or optimized to achieve enhanced, pharmacokinetics or potency. In some instances, a peptide described herein can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 150, or about at least 200 amino acids in length. In some instances, a peptide described herein can be from about 1 to about 48, from about 2 to about 48, from about 3 to about 48, from about 4 to about 48, from about 5 to about 48, from about 6 to about 48, from about 7 to about 48, from about 8 to about 48, from about 9 to about 48, from about 10 to about 48, from about 11 to about 48, from about 12 to about 48, from about 13 to about 48, from about 14 to about 48, from about 15 to about 48, from about 16 to about 48, from about 17 to about 48, from about 18 to about 48, from about 19 to about 48, from about 20 to about 48, from about 21 to about 48, from about 22 to about 48, from about 23 to about 48, from about 24 to about 48, from about 25 to about 48, from about 26 to about 48, from about 27 to about 48, from about 28 to about 48, from about 29 to about 48, from about 30 to about 48, from about 31 to about 48, from about 32 to about 48, from about 33 to about 48, from about 34 to about 48, from about 35 to about 48, from about 36 to about 48, from about 37 to about 48, from about 38 to about 48, from about 39 to about 48, from about 40 to about 48, from about 41 to about 48, from about 42 to about 48, from about 43 to about 48, from about 44 to about 48, from about 45 to about 48, from about 46 to about 48, or from about 47 to about 48 amino acids in length.

Salts

A peptide disclosed herein can be a salt thereof. In some instances, recitation of the phrases “peptide” or “polypeptide” should be construed to include a salt thereof even if not explicitly recited.

In some instances, a salt can include a carboxylate salt (e.g. formate, acetate, trifluoroacetate, trichloroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, pamoate, nicotinate, isonicotinate, cinnamate, oxalate, malonate, succinate, suberate, sebacate, fumarate, malate, maleate, hydroxymaleate, hippurate, phthalate or a terephthalate salts); a halide salt (e.g. chloride, bromide or iodide salts); a sulfonate salt (e.g. benzene sulfonate, methyl-, bromo- or chloro-benzenesulfonate, xylenesulfonate, methanesulfonate, trifluoromethanesulfonate, ethanesulfonate, propanesulfonate, hydroxyethanesulfonate, 1- or 2-naphthalene-sulfonate or 1,5-naphthalenedisulfonate salts); a sulfate salt; a pyrosulfate salt; a bisulfate salt; a sulfite salt; a bisulfite salt; a phosphate salt; a monohydrogenphosphate salt; a dihydrogenphosphate salt; a metaphosphate salt; a pyrophosphate salt; a nitrate salt; and the like.

In some instances, a salt can be a pharmaceutically acceptable salt. In some instances, a pharmaceutically acceptable salt can be a salt described in Berge et al, J. Pharm. Sci, 1977. In some instances, a pharmaceutically acceptable salts can include those salts prepared by reaction of a peptide with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

Amino Acids

In some instances, amino acids can be canonical amino acids such as the 20 proteinogenic L-amino acids. In some instances, amino acids can be unnatural amino acids. An “unnatural amino acid” as described herein can include any amino acid other than one of the 20 proteinogenic proteins in an L-configuration. Such amino acids can include amino acids with non-canonical side chains, D-amino acids, β-amino acids, and the like. Exemplary amino acids described below are depicted in the L-configuration, but can be a configuration other than an L-configuration.

In some instances, an unnatural amino acid can be an NMR-promoting agent. An unnatural amino acid for use as an NMR promoting agent can comprise an amino acid with an NMR active side chain, or a side chain capable of becoming NMR active. In some instances, an NMR-promoting agent can be selected from the group consisting of a spin-labeled compound, a paramagnetic metal chelating compound, a compound comprising an NMR active isotope, and any combination thereof.

In some cases, a spin-labeled compound can be prepared through reaction of an amino acid such asp-acetylphenylalanine with a nitroxide compound:

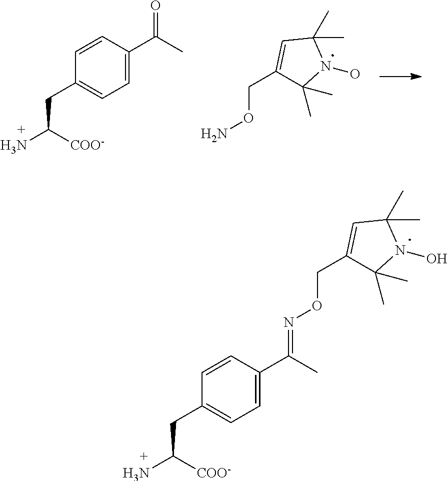

In some instances, a spin labeled compound can be 4-(3,3,5,5-tetramethyl-2,6-dioxo-4-oxylpiperazin-1-yl)-L-phenylglycine (TOPP).

In some instances, a paramagnetic metal chelating compound can include an amino acid comprising a side chain of bipyridine or hydroxyquinoline.

In some instances, an amino acid described herein can comprise an NMR active isotope. Examples can include ¹⁵N, ¹³C, and ³¹P.

In some instances, an unnatural acid can be a fluorescent amino acid comprising a fluorescent side chain. Examples can include derivative of coumarin, fluorescein, and the like.

In some instances, a peptide described herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100 or 150 amino acids in a D-configuration. In some instances, a peptide described herein can comprise from about 1 to about 48, from about 2 to about 48, from about 3 to about 48, from about 4 to about 48, from about 5 to about 48, from about 6 to about 48, from about 7 to about 48, from about 8 to about 48, from about 9 to about 48, from about 10 to about 48, from about 11 to about 48, from about 12 to about 48, from about 13 to about 48, from about 14 to about 48, from about 15 to about 48, from about 16 to about 48, from about 17 to about 48, from about 18 to about 48, from about 19 to about 48, from about 20 to about 48, from about 21 to about 48, from about 22 to about 48, from about 23 to about 48, from about 24 to about 48, from about 25 to about 48, from about 26 to about 48, from about 27 to about 48, from about 28 to about 48, from about 29 to about 48, from about 30 to about 48, from about 31 to about 48, from about 32 to about 48, from about 33 to about 48, from about 34 to about 48, from about 35 to about 48, from about 36 to about 48, from about 37 to about 48, from about 38 to about 48, from about 39 to about 48, from about 40 to about 48, from about 41 to about 48, from about 42 to about 48, from about 43 to about 48, from about 44 to about 48, from about 45 to about 48, from about 46 to about 48, or from about 47 to about 48 amino acids in a D-configuration. In some instances, a peptide described herein may not comprise an amino acid in a D-configuration.

In some instances, a peptide described herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in an L-configuration. In some instances, a peptide described herein can comprise from about 1 to about 48, from about 2 to about 48, from about 3 to about 48, from about 4 to about 48, from about 5 to about 48, from about 6 to about 48, from about 7 to about 48, from about 8 to about 48, from about 9 to about 48, from about 10 to about 48, from about 11 to about 48, from about 12 to about 48, from about 13 to about 48, from about 14 to about 48, from about 15 to about 48, from about 16 to about 48, from about 17 to about 48, from about 18 to about 48, from about 19 to about 48, from about 20 to about 48, from about 21 to about 48, from about 22 to about 48, from about 23 to about 48, from about 24 to about 48, from about 25 to about 48, from about 26 to about 48, from about 27 to about 48, from about 28 to about 48, from about 29 to about 48, from about 30 to about 48, from about 31 to about 48, from about 32 to about 48, from about 33 to about 48, from about 34 to about 48, from about 35 to about 48, from about 36 to about 48, from about 37 to about 48, from about 38 to about 48, from about 39 to about 48, from about 40 to about 48, from about 41 to about 48, from about 42 to about 48, from about 43 to about 48, from about 44 to about 48, from about 45 to about 48, from about 46 to about 48, or from about 47 to about 48 amino acids in an L-configuration. In some instances, a peptide described herein may not comprise an amino acid in an L-configuration.

In some instances, a peptide described herein can comprise only canonical amino acids. In some instances, a peptide can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75 or 150 amino acids that may not be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some instances, a peptide described herein can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, or 150 unnatural amino acids.

As used herein, the symbol “X” can refer to an amino acid that can be independently Gly, or an amino acid that can comprise a C₁-C₁₀ alkyl, C₁-C₁₀ alkenyl, C₁-C₁₀ alkynyl, cycloalkyl, or alkylcycloalkyl side chain. In some cases, this can include canonical amino acids such as glycine, alanine, valine, leucine and isoleucine. In some cases, this can include non-standard amino acids. Exemplary amino acids are depicted below:

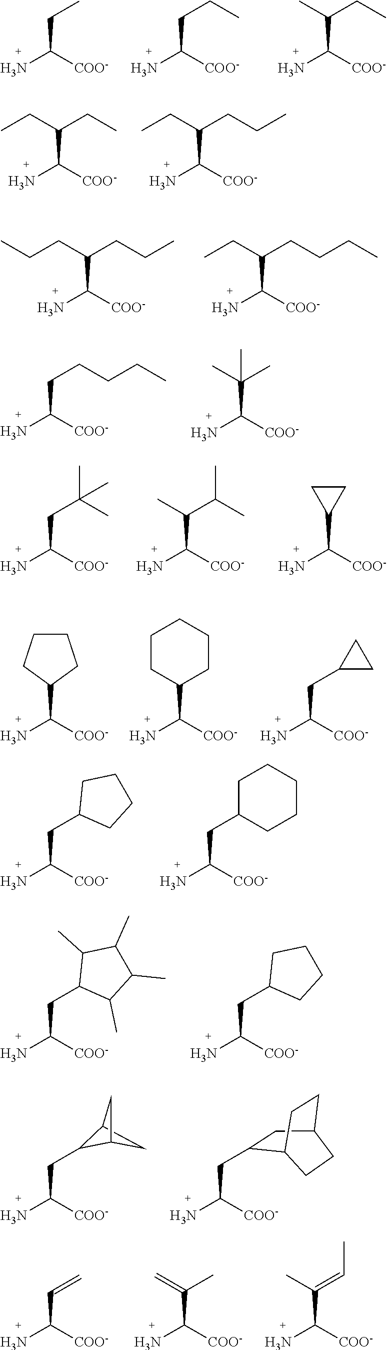

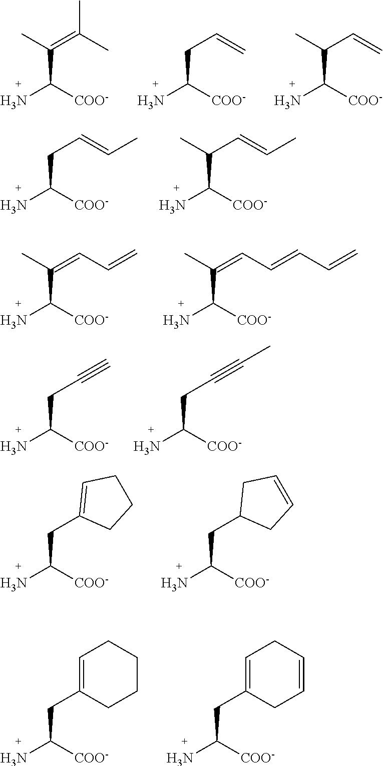

As used herein, the symbol “Ar” can refer to an amino acid that can comprise an aromatic side chain. In some cases, this can include canonical amino acids such as phenylalanine, tyrosine, tryptophan, and histidine. In some cases, this can include non-standard amino acids. Exemplary amino acids are depicted below:

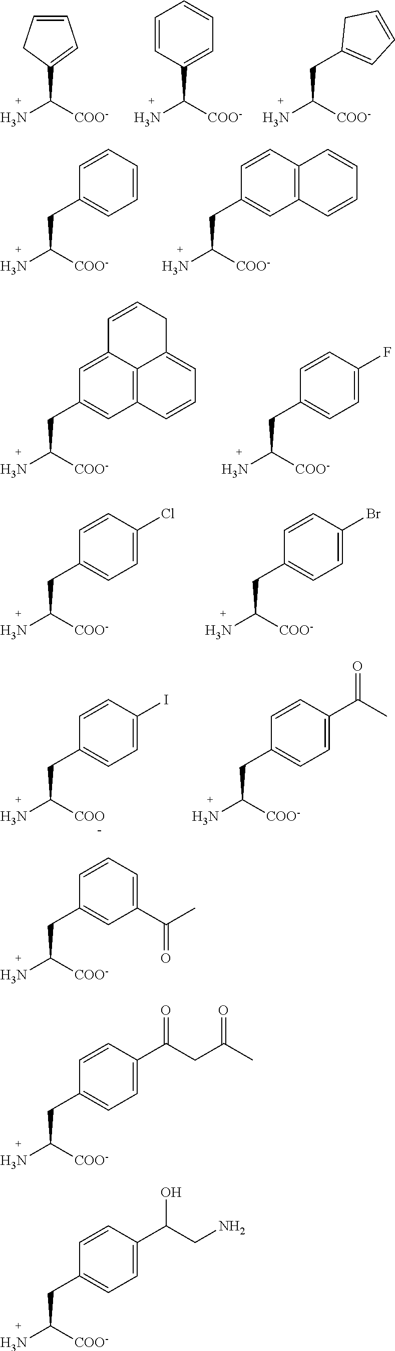

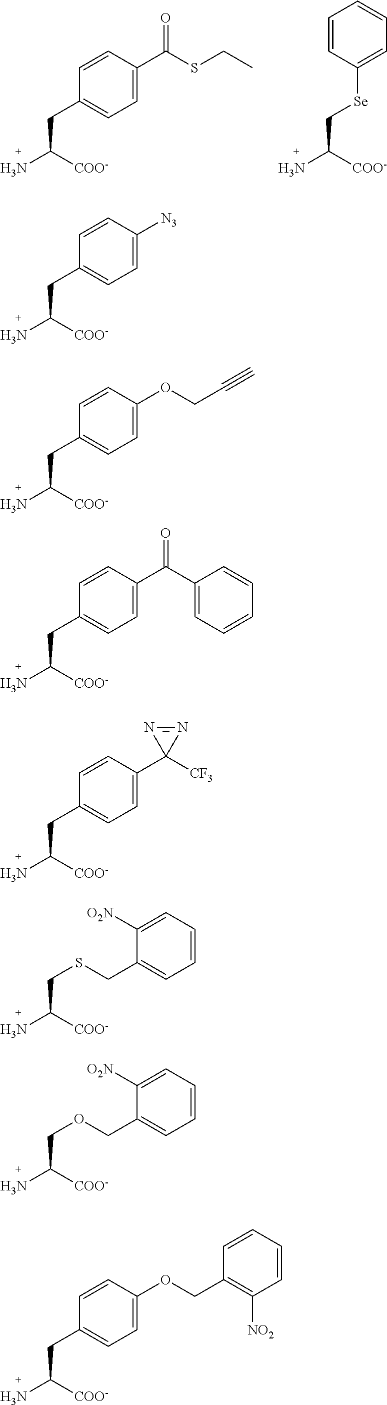

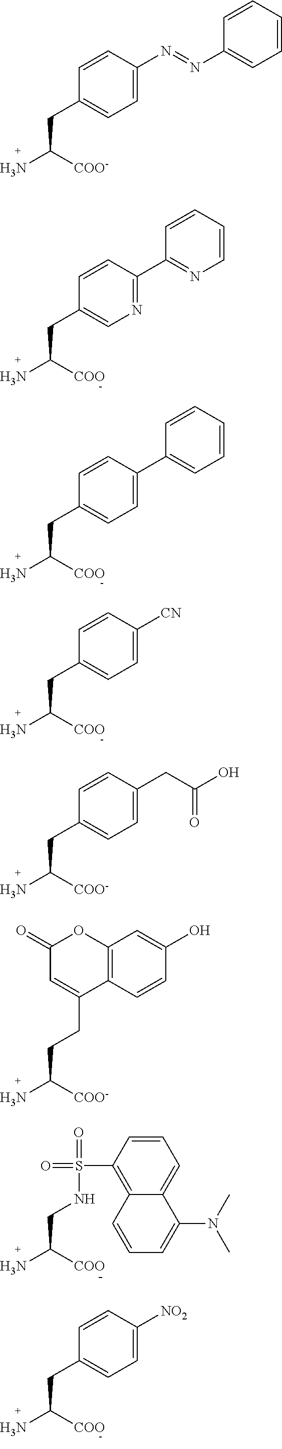

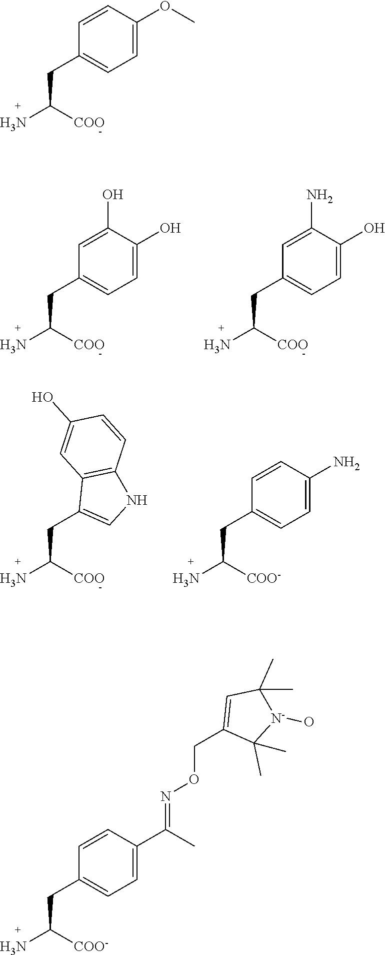

As used herein, the symbol “Y” can refer to an amino acid that can comprise a side chain that can be at least partially protonated at a pH of about 7.3. In some cases, this can include canonical amino acids such as lysine, arginine, and histidine. In some cases, this can include non-standard amino acids. Exemplary amino acids are depicted below:

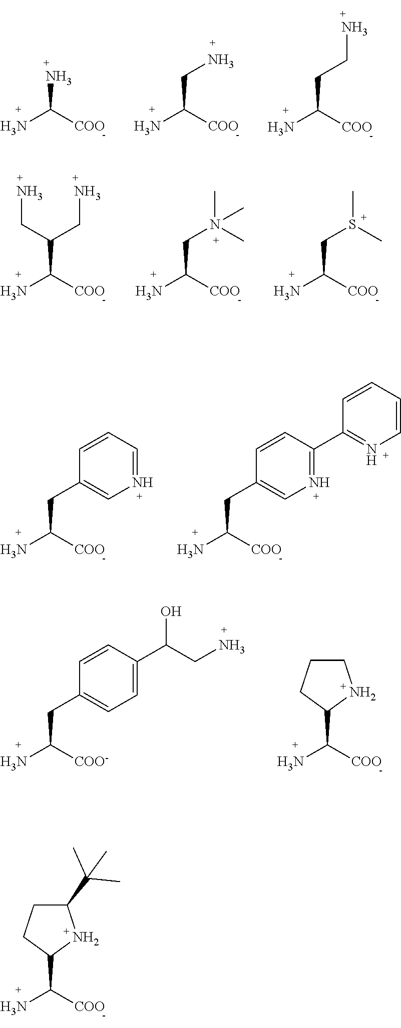

As used herein, the symbol “U” can refer to an amino acid that can comprise an amide containing side chain. In some cases, this can include canonical amino acids such as glutamine and asparagine. In some cases, this can include non-standard amino acids. Exemplary amino acids are depicted below:

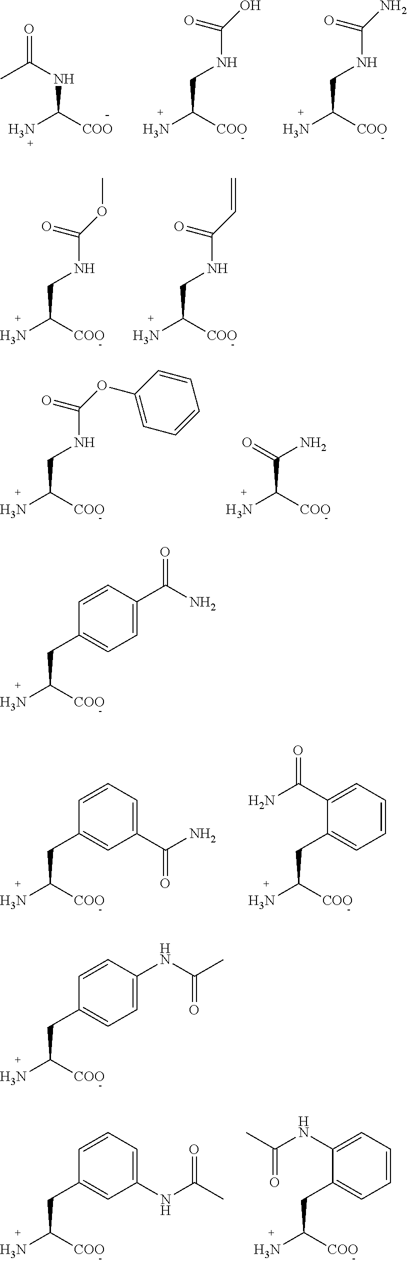

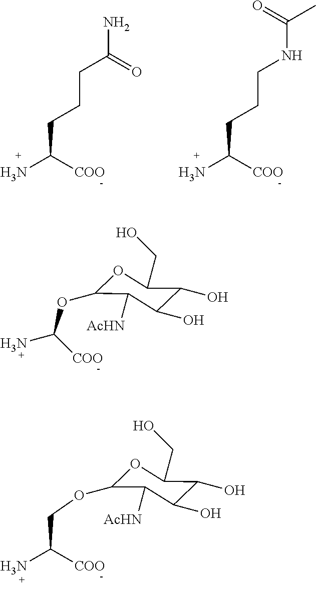

As used herein, the symbol “$” can refer to an amino acid that can comprise an alcohol or thiol containing side chain. In some cases, this can include canonical amino acids such as serine, threonine, tyrosine, cysteine, and methionine. In some cases, this can include non-standard amino acids. Exemplary amino acids are depicted below:

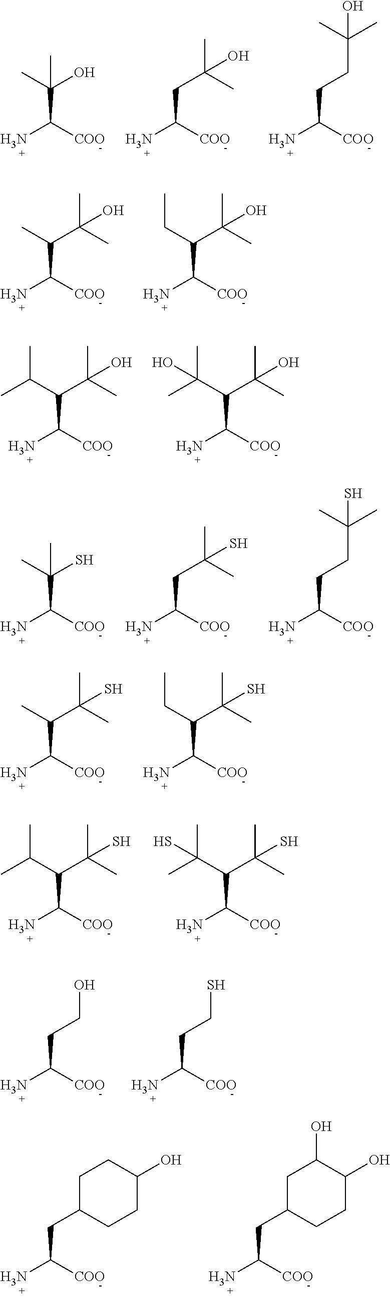

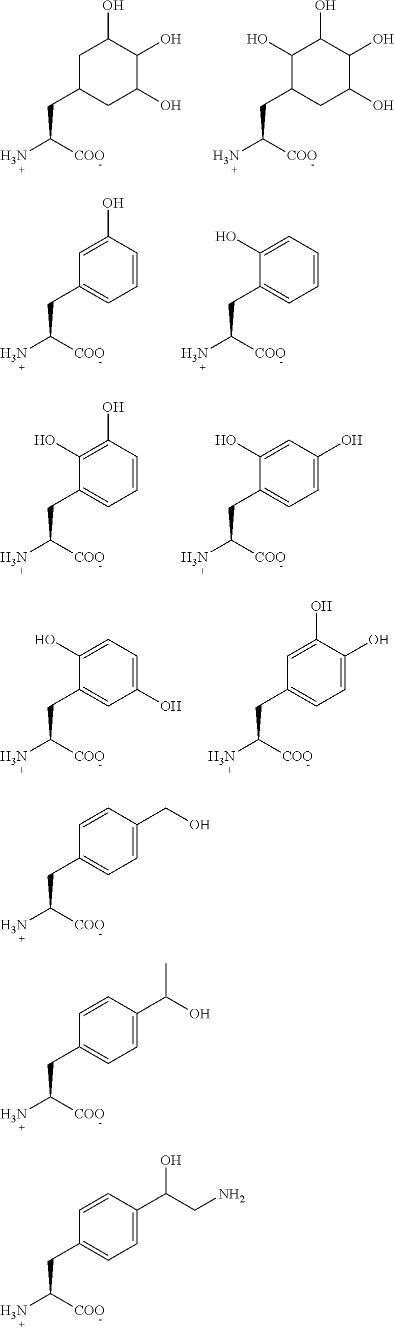

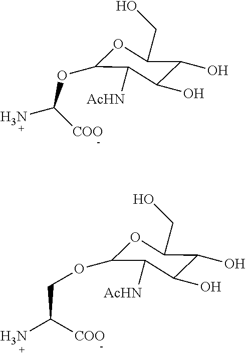

As used herein, the symbol “@” can refer to an amino acid that can comprise a side chain that can be at least partially deprotonated at a pH of about 7.3. In some cases, this can include canonical amino acids such as glutamate and aspartate. In some cases, this can include non-standard amino acids. Exemplary amino acids are depicted below:

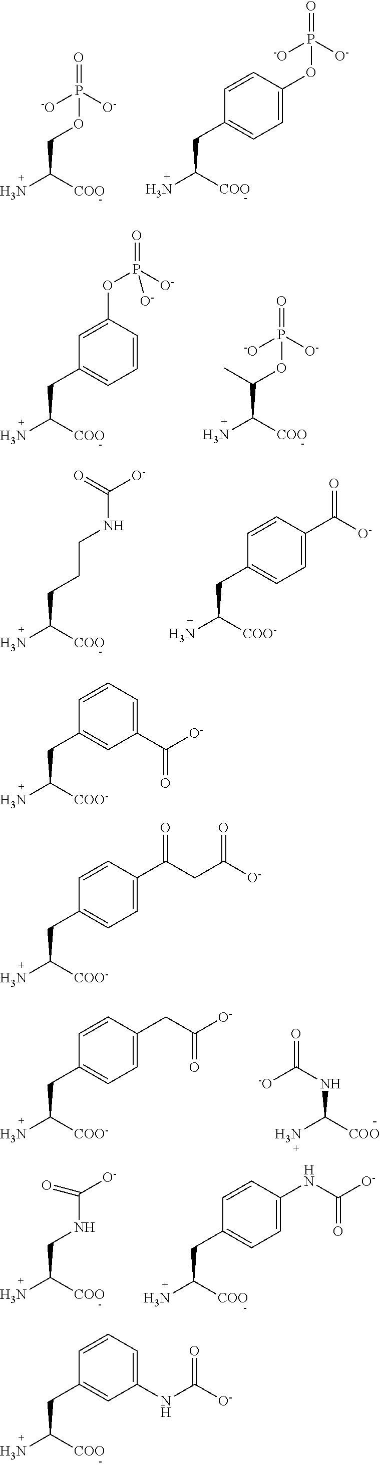

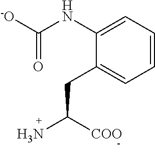

Polypeptide Formulas

In some instances, a peptide described herein can comprise a polypeptide sequence of general formula (AA₁-AA₂-AA₃-AA₄-AA₅-AA₆-AA₇)ₙ, where n can be a number ranging from about 1 to about 7. In some instances, n can be about at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 8.0, 9.0 or 10.0.

In some cases, the distribution of polar, charged, and non-polar/aromatic residues can be modulated to adjust the amphipathicity or charge distribution of a peptide. A polypeptide described herein can comprise a polypeptide sequence of Formula A, Formula B, Formula C, Formula D, Formula E, Formula F, Formula G, Formula H, Formula I, Formula J, Formula K, Formula L, Formula M, Formula N, or a salt of any of these. In some cases, at least one AA₁ can be an N-terminal amino acid. In some cases, an amino group of an N-terminal amino acid can comprise substituents R′ and R″, where: R′ and R″ can be independently H; phosphoryl; alkyl; alkenyl; alkynyl; cycloalkyl; sulfonyl; sulfinyl; silyl; a fatty acid; pyroglutamyl; an isocyanate; an alkyl carbonyl which can be substituted with a halogen, an alkyl group, a cylcloalkyl group, or any combination thereof; a thioester, acetyl, a urea, a carbamate, a sulfonamide, an alkylamine, aryl, alkylaryl, a heteroaryl, alkyheteroaryl; or RC(O)—; where R can be independently H, D, alkyl, cycloalkyl, aryl, heteroaryl, alkylaryl, heteroaryl, or alkyheteroaryl; or R′ and R″ together with the nitrogen atom to which they are attached, form a substituted or non-substituted 5, 6, or 7-membered ring.

In some cases, a peptide discloses herein may not comprise 3 or more contiguous arginine or lysine residues. In some cases, a peptide may not be a cyclic peptide. In some cases, at least one, two, or all of the following can apply to a peptide disclosed herein: (i) a peptide or salt thereof can exhibit antimicrobial activity against a bacteria with a minimum inhibitory concentration ranging from about 0.1 μg/mL to about 100 μg/mL in vitro; (ii) a peptide or salt thereof can exhibit antiviral activity against a virus with a minimum inhibitory concentration ranging from about 0.1 μg/mL to about 100 μg/mL in vitro; or (iv) a peptide or salt thereof can exhibit antitumor activity against a tumor cell with an LD50 of from about 0.01 μM to about 100 μM in vitro.

In some cases, a polypeptide can be a polypeptide of Formula A: (AA₁-AA₂-AA₃-AA₄-AA₅-AA₆AA₇)ₙ, where: AA₁ can be independently X, Ar, or Y; and AA₂, AA₃, AA₄, AA₅, AA₆, and AA₇ can be independently Y, U, $ or @.

In some cases, a polypeptide can be a polypeptide of Formula B: (AA₁-AA₂-AA₃-AA₄-AA₅-AA₆AA₇)ₙ, where: AA₁ and AA₅ can be independently X, Y, or Ar; and AA₂, AA₃, AA₄, AA₆, and AA₇ can be independently Y, U, $ or @.

In some cases, a polypeptide can be a polypeptide of Formula C: (AA₁-AA₂-AA₃-AA₄-AA₅-AA₆AA₇)ₙ, where: AA₁ and AA₄ can be independently X, Y, or Ar; and AA₂, AA₃, AA₅, AA₆, and AA₇ can be independently Y, U, $ or @.

In some cases, a polypeptide can be a polypeptide of Formula D: (AA₁-AA₂-AA₃-AA₄-AA₅-AA₆AA₇)ₙ, where: AA₁ can be independently X, Y, or Ar; AA₄ and AA₅ can be independently X or Ar; AA₂ and AA₇ can be independently U, $ or @; and AA₃ and AA₆ can be independently Y, U, $ or @.

In some cases, a polypeptide can be a polypeptide of Formula E: (AA₁-AA₂-AA₃-AA₄-AA₅-AA₆AA₇)ₙ, where: AA₁ can be independently X, Y, or Ar; AA₂, AA₄, and AA₅ can be independently X or Ar; and AA₃, AA₆, and AA₇ can be independently Y, U, $ or @.

In some cases, a polypeptide can be a polypeptide of Formula F: (AA₁-AA₂-AA₃-AA₄-AA₅-AA₆AA₇)ₙ, where: AA₁ can be independently X, Y, or Ar; AA₄, AA₅, and AA₇ can be independently X or Ar; and AA₂, AA₃, and AA₆ can be independently Y, U, $ or @.

In some cases, a polypeptide can be a polypeptide of Formula G: (AA₁-AA₂-AA₃-AA₄-AA₅-AA₆AA₇)ₙ, where: AA₁, AA₄, AA₅ can be independently X, Y, or Ar; AA₂ and AA₇ can be independently X or Ar; and AA₃ and AA₆ can be independently Y, U, $ or @.

In some cases, a polypeptide can be a polypeptide of Formula H: (AA₁-AA₂-AA₃-AA₄-AA₅-AA₆AA₇)ₙ, where: AA₁ can be independently Y, U, $, or @; AA₃, AA₄, AA₅, and AA₆ can be independently X, Y, or Ar; and AA₂ and AA₇ can be independently X or Ar.

In some cases, a polypeptide can be a polypeptide of Formula I: (AA₁-AA₂-AA₃-AA₄-AA₅-AA₆AA₇)ₙ, where: AA₁ and AA₅ can be independently Y, U, $, or @; AA₃, AA₄, and AA₆ can be independently X, Y, or Ar; and AA₂ and AA₇ can be independently X or Ar.

In some cases, a polypeptide can be a polypeptide of Formula J: (AA₁-AA₂-AA₃-AA₄-AA₅-AA₆AA₇)ₙ, where: AA₁ and AA₄ can be independently Y, U, $, or @; AA₃, AA₅, and AA₆ can be independently X, Y, or Ar; and AA₂ and AA₇ can be independently X or Ar.

In some cases, a polypeptide can be a polypeptide of Formula K: (AA₁-AA₂-AA₃-AA₄-AA₅-AA₆AA₇)ₙ, where: AA₁, AA₄, and AA₅ can be independently Y, U, $, or @; and AA₂, AA₃, AA₆, and AA₇ can be independently X, Y, or Ar.

In some cases, a polypeptide can be a polypeptide of Formula L: (AA₁-AA₂-AA₃-AA₄-AA₅-AA₆AA₇)ₙ, where: AA₁, AA₂, AA₄, and AA₅ can be independently Y, U, $, or @; and AA₃, AA₆, and AA₇ can be independently X, Y, or Ar.

In some cases, a polypeptide can be a polypeptide of Formula M: (AA₁-AA₂-AA₃-AA₄-AA₅-AA₆AA₇)ₙ, where: AA₁, AA₄, AA₅, and AA₇ can be independently Y, U, $, or @; and AA₂, AA₃, and AA₆ can be independently X, Y, or Ar.

In some cases, a polypeptide can be a polypeptide of Formula N: (AA₁-AA₂-AA₃-AA₄-AA₅-AA₆AA₇)ₙ, where: AA₁, AA₂, AA₄, AA₅, and AA₇ can be independently Y, U, $, or @; and AA₃ and AA₆ can be independently X, Y, or Ar.

In some exemplary embodiments, a peptide or salt thereof described herein can comprise a polypeptide sequence of formula [Y-Ar-X-Y-Y-X-X]ₙ. In some exemplary embodiments, a peptide or salt thereof described herein can comprise a polypeptide sequence of formula [U-Ar-X-Y-Y-X-Ar]ₙ. In some exemplary embodiments, a peptide or salt thereof described herein can comprise a polypeptide sequence of formula [Y-X-X-$-$-X-X]n. In some exemplary embodiments, a peptide or salt thereof described herein can comprise a polypeptide sequence of formula [Y-X-X-$-$-X-X-@-X-X-$-$-X-X]ₙ.

In some cases, a polypeptide can be a rational variant of a polypeptide based on an LLP scaffold. In some instances, a polypeptide can be of sequence:

(i)
Y-X-X-Y-X-X-Y-Y-X-X-Y-Y;
(ii)
Y-X-X-Y-X-X-Y-Y-Ar-X-Y-Y;
(iii)
Y-Ar-Ar-Y-Ar-Ar-Y-Y-Ar-Ar-Y-Y;
(iv)
Ar-Y-Y-Ar-Ar-Y-Y-Ar-Ar-Y-Ar-Ar-Y-Y-Ar-Ar-Y-Y;
(v)
Y-Y-X-X-Y-Y-X-Y-Y-X-X-Y-Y-X-X-Y-X-X-Y-Y-X-X-Y-Y;
(vi)
Y-Y-Ar-X-Y-Y-X-Y-Y-X-Ar-Y-Y-X-X-Y-X-X-Y-Y-Ar-X-Y-
Y;
(vii)
Y-Y-Ar-Ar-Y-Y-Ar-Y-Y-Ar-Ar-Y-Y-Ar-Ar-Y-Ar-Ar-Y-Y-
Ar-Ar-Y-Y;
(viii)
X-Y-Y-X-X-Y-Y-X-X-Y-X-X-Y-Y-X-X-Y-Y-X-Y-Y-X-X-Y-
Y-X-X-Y-X-X-Y-Y-X-X-Y-Y;
(ix)
X-Y-Y-X-Ar-Y-Y-X-X-Y-X-X-Y-Y-Ar-X-Y-Y-X-Y-Y-X-Ar-
Y-Y-X-X-Y-X-X-Y-Y-Ar-X-Y-Y;
(x)
Y-Y-X-X-Y-Y-X-Y-Y-X-X-Y-Y-X-X-Y-X-X-Y-Y-X-X-Y-Y-
X-Y-Y-X-X-Y-Y-X-X-Y-X-X-Y-Y-X-X-Y-Y;
(xi)
Y-X-X-Y-X-X-Y-Y-X-X-Y-Y-X-Y-Y-X-X-Y-Y-X-X-Y-X-X-
Y-Y-X-X-Y-Y-X-Y-Y-X-X-Y-Y-X-X-Y-X-X-Y-Y-X-X-Y-Y;
or
(xii)
Y-X-X-Y-X-X-Y-Y-Ar-X-Y-Y-X-Y-Y-X-Ar-Y-Y-X-X-Y-X-
X-Y-Y-Ar-X-Y-Y-X-Y-Y-X-Ar-Y-Y-X-X-Y-X-X-Y-Y-Ar-Y-
X-X;

where a peptide or salt thereof contains at least one amino acid that may not be Val, Trp or Arg. In some aspects, the peptide may not be a cyclic peptide.

In some specific embodiments, a peptide or salt thereof can comprise from about 60% to about 70%, from about 60% to about 80%, from about 60% to about 90%, from about 60% to about 91%, from about 60% to about 95%, or from about 60% to about 100% homology to a LLP homolog sequence selected from the group consisting of:

(i)
(SEQ ID NO: 15)
Arg-Val-Val-Arg-Val-Val-Arg-Arg-Val-Val-Arg-Arg;
(ii)
(SEQ ID NO: 16)
Arg-Val-Val-Arg-Val-Val-Arg-Arg-Trp-Val-Arg-Arg;
(iii)
(SEQ ID NO: 17)
Arg-Trp-Trp-Arg-Trp-Trp-Arg-Arg-Trp-Trp-Arg-Arg;
(iv)
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(v)
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(vi)
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(vii)
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(viii)
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(ix)
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(x)
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(xi)
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
and
(xii)
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val.

In some specific embodiments, A peptide can comprise about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% homology to a LLP homolog sequence selected from the group consisting of:

(i)
(SEQ ID NO: 15)
Arg-Val-Val-Arg-Val-Val-Arg-Arg-Val-Val-Arg-Arg;
(ii)
(SEQ ID NO: 16)
Arg-Val-Val-Arg-Val-Val-Arg-Arg-Trp-Val-Arg-Arg;
(iii)
(SEQ ID NO: 17)
Arg-Trp-Trp-Arg-Trp-Trp-Arg-Arg-Trp-Trp-Arg-Arg;
(iv)
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(v)
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(vi)
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(vii)
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(viii)
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(ix)
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(x)
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(xi)
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
and
(xii)
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val.

In some embodiments, a peptide disclosed herein may not comprise three or more contiguous arginine or lysine residues. In some instances, a peptide disclosed herein can comprise 0, 1, 2, 3, 4, 5, 6, 7, 8, 9, 10, 11, 12, 13, 14, 15, 16, 17, 18, 19, or 20 Arg. In some instances, a peptide disclosed herein can comprise 0, 1, 2, 3, 4, 5, 6, 7, 8, 9, 10, 11, 12, 13, 14, 15, 16, 17, 18, 19, or 20 Lys. In some instances, a peptide disclosed herein can comprise 0, 1, 2, 3, 4, 5, 6, 7, 8, 9, 10, 11, 12, 13, 14, 15, 16, 17, 18, 19, or 20 His. In some instances, a peptide disclosed herein can comprise 0, 1, 2, 3, 4, 5, 6, 7, 8, 9, 10, 11, 12, 13, 14, 15, 16, 17, 18, 19, or 20 Ile. In some instances, a peptide disclosed herein can comprise 0, 1, 2, 3, 4, 5, 6, 7, 8, 9, 10, 11, 12, 13, 14, 15, 16, 17, 18, 19, or 20 Leu. In some instances, a peptide disclosed herein can comprise 0, 1, 2, 3, 4, 5, 6, 7, 8, 9, 10, 11, 12, 13, 14, 15, 16, 17, 18, 19, or 20 Met. In some instances, a peptide disclosed herein can comprise 0, 1, 2, 3, 4, 5, 6, 7, 8, 9, 10, 11, 12, 13, 14, 15, 16, 17, 18, 19, or 20 Phe. In some instances, a peptide disclosed herein can comprise 0, 1, 2, 3, 4, 5, 6, 7, 8, 9, 10, 11, 12, 13, 14, 15, 16, 17, 18, 19, or 20 Thr. In some instances, a peptide disclosed herein can comprise 0, 1, 2, 3, 4, 5, 6, 7, 8, 9, 10, 11, 12, 13, 14, 15, 16, 17, 18, 19, or 20 Trp. In some instances, a peptide disclosed herein can comprise 0, 1, 2, 3, 4, 5, 6, 7, 8, 9, 10, 11, 12, 13, 14, 15, 16, 17, 18, 19, or 20 Val. In some instances, a peptide disclosed herein can comprise 0, 1, 2, 3, 4, 5, 6, 7, 8, 9, 10, 11, 12, 13, 14, 15, 16, 17, 18, 19, or 20 Cys. In some instances, a peptide disclosed herein can comprise 0, 1, 2, 3, 4, 5, 6, 7, 8, 9, 10, 11, 12, 13, 14, 15, 16, 17, 18, 19, or 20 Gln. In some instances, a peptide disclosed herein can comprise 0, 1, 2, 3, 4, 5, 6, 7, 8, 9, 10, 11, 12, 13, 14, 15, 16, 17, 18, 19, or 20 Gly. In some instances, a peptide disclosed herein can comprise 0, 1, 2, 3, 4, 5, 6, 7, 8, 9, 10, 11, 12, 13, 14, 15, 16, 17, 18, 19, or 20 Pro. In some instances, a peptide disclosed herein can comprise 0, 1, 2, 3, 4, 5, 6, 7, 8, 9, 10, 11, 12, 13, 14, 15, 16, 17, 18, 19, or 20 Ser. In some instances, a peptide disclosed herein can comprise 0, 1, 2, 3, 4, 5, 6, 7, 8, 9, 10, 11, 12, 13, 14, 15, 16, 17, 18, 19, or 20 Tyr. In some instances, a peptide disclosed herein can comprise 0, 1, 2, 3, 4, 5, 6, 7, 8, 9, 10, 11, 12, 13, 14, 15, 16, 17, 18, 19, or 20 Ala. In some instances, a peptide disclosed herein can comprise 0, 1, 2, 3, 4, 5, 6, 7, 8, 9, 10, 11, 12, 13, 14, 15, 16, 17, 18, 19, or 20 Asn. In some instances, a peptide disclosed herein can comprise 0, 1, 2, 3, 4, 5, 6, 7, 8, 9, 10, 11, 12, 13, 14, 15, 16, 17, 18, 19, or 20 Asp. In some instances, a peptide disclosed herein can comprise 0, 1, 2, 3, 4, 5, 6, 7, 8, 9, 10, 11, 12, 13, 14, 15, 16, 17, 18, 19, or 20 Glu.

In some instances, a peptide disclosed herein may not be cyclic. In other instances, a peptide disclosed herein can be a cyclic peptide.

Exemplary peptides are depicted in Table 1 below:

| SEQ ID | |
|---|---|
| NO: | Amino Acid Sequence |
| 1 | RRWVRRVRRVWRRVVRVVRRWVRR |
| 2 | IRRRRRRIRRRRRR |
| 3 | IRRRIRRIRRRIRRIRRRIRR |
| 4 | IRRIIRRIRRIIRRIRRIIRR |
| 5 | VWRWVRRVWRWVRRVWRWVRR |
| 6 | VWRWVRRVWRWVRR |
| 7 | VVRVVRRVVRVVRR |
| 8 | VVRVVRVVVRVVRVVVRVVRV |
| 9 | RSRVVRSWSRV |
| 10 | RFVRRVRRFVRRVRRFVRRVRRFVRRVRRFVRRVRRFVRRVRR |
| | FVRRVRRFVRRVRRFVRRVRRFVRRVRRFVRRVRRFVRRVR |
| 11 | RRTYSRSRRTYSRSRRTYSR |
| 12 | KVVSSHETISSVVKVVSSHETISSVV |
| 13 | KKTHTKTKKTHTKTKKTHTK |
| 14 | VVRVVRRVVRVVRRVVRVVRR |

In some specific embodiments, a peptide disclosed herein can comprise any one of SEQ ID NO:1 to SEQ ID NO:13.

A peptide can comprise from about 60% to about 70%, from about 60% to about 80%, from about 60% to about 90%, from about 60% to about 91%, from about 60% to about 95%, or from about 60% to about 100% homology to a peptide of any one of SEQ ID NO:1 to SEQ ID NO:13. A peptide can comprise about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% homology to a peptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13.

Synthesis of Peptides

Skilled artisans are aware of many suitable methods available for synthesizing peptides. Skilled artisans are aware of multiple methods for culturing recombinant cells to produce (and optionally secrete) a peptide as disclosed herein, as well as for purification and/or isolation of expressed peptide. The methods chosen for protein purification can depend on many variables, including the properties of a protein of interest, its location and form within a cell, the vector, host strain background, and the intended application for an expressed protein. Culture conditions can also have an effect on solubility and localization of a given target protein. Many approaches can be used to purify target proteins expressed in recombinant microbial cells as disclosed herein, including without limitation ion exchange and gel filtration.

In some instances, a peptide fusion tag can be added to a recombinant protein. Some peptide fusion tags such as maltose-binding protein (MBP), thioredoxin (Trx), glutathione-S-transferase (GST), poly-histidine, and chitin binding protein (CBP) can be utilized for a variety of affinity purification methods that take advantage of a peptide fusion tag. In some cases, the use of an affinity method can enable the purification of a target protein to near homogeneity in one step. Purification may include cleavage of part or all of a fusion tag with enterokinase, factor Xa, thrombin, or HRV 3C proteases, for example. In some instances, before purification or activity measurements of an expressed target protein, preliminary analysis of expression levels, cellular localization, and solubility of a target protein can be performed. A target protein may be found in any or all of the following fractions: soluble or insoluble cytoplasmic fractions, periplasm, or medium.

Epitope fusion tags can be fused to an N- or C-terminus of a peptide described herein in order to detect protein levels of a protein through a visualization method such as western blot, immunofluorescence, or immunoprecipitation. Examples can include a VHS-tag, a Myc-tag, an HA-tag, a FLAG-tag, an NE-tag, and the like.

In some cases, a fluorescent protein can be fused to an N- or C-terminus of a peptide described herein. In some cases, a fluorescent protein can be employed as a folding reporter protein in order to determine whether a particular protein scaffold is properly folded. In some cases, a fluorescent protein can be employed as a marker to allow for imaging of a fusion protein when administered to a subject. Examples can include fluorescent proteins such as green fluorescent protein (GFP), Emerald, Superfolder GFP, folding-reporter GFP, Azami Green, mWasabi, TagGFP, TurboGFP, enhanced GFP (eGFP), ZsGreen, T-Sapphire, blue-fluorescent protein (BFP), enhanced BFP (eBFP), eBFP2, Azurite, Cerulean, yellow-fluorescent protein (YFP), eYFP, Topaz, Venus, mCitrine, YPet, TagYFP, ZsYellow, PhiYFP, ZsYellow, mBanana, orange fluorescent protein (OFP), Kusabira Orange, Kusabira Orange2, mOrange, mOrange2, dTomato, mTangerine, red fluorescent protein (RFP), mRuby, mApple, mStrawberry, AsRed2, JRed, mRaspberry, dKeima-tandem, mPlum, HcRed-tandem, mCherry, mTurquoise, cyano-fluorescent protein (CFP) and the like.

In some instances, a peptide disclosed herein can be synthesized chemically without the use of a recombinant production system. Protein synthesis can be carried out in a liquid-phase system or in a solid-phase system using techniques known in the art (see, e.g., Atherton, E., Sheppard, R. C. (1989). Solid Phase peptide synthesis: a practical approach. Oxford, England: IRL Press; Stewart, J. M., Young, J. D. (1984). Solid phase peptide synthesis (2nd ed.). Rockford: Pierce Chemical Company). In some cases, a peptide can be chemically synthesized with an identification tag as described in U.S. Pat. No. 4,703,004. Peptides described herein can also be synthesized by techniques such as native chemical ligation, as described in U.S. Pat. No. 6,184,344.

### IV. Formulations

At least one peptide disclosed herein can be formulated as a pharmaceutical formulation. In some embodiments, a pharmaceutical formulation can comprise 1, 2, 3, 4, 5, 6, 7, 8, 9, 10, 11, 12, 13, 14, 15, 16, 17, 18, 19, 20 or more peptides disclosed herein. In some embodiments a pharmaceutical formulation can comprise 0, 1, 2, 3, 4, 5, 6, 7, 8, 9, 10, 11, 12, 13, 14, 15, 16, 17, 18, 19, 20 or more additional peptides or proteins. In some instances, a pharmaceutical formulation can comprise a peptide described herein and at least one of: an excipient, a diluent, or a carrier.

In some embodiments, a pharmaceutical formulation can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminium hydroxide, sodium citrate; sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A; TL-3, sodium ortho vanadate, sodium fluoride; N-a-tosyl-Phe-chloromethylketone, N-a-tosyl-Lys-chloromethylketone, aprotinin, phenyl methylsulfonyl fluoride, diisopropylfiuorophosphate, kinase inhibitor, phosphatase inhibitor, caspase inhibitor, granzyme inhibitor, cell adhesion inhibitor, cell division inhibitor, cell cycle inhibitor, lipid. signaling inhibitor, protease inhibitor, reducing agent, alkylating agent, antimicrobial agent, oxidase inhibitor, or other inhibitor.

In some embodiments a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical formulation can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical formulation can comprise a coloring agent. Non-limiting examples of suitable color agents can include food, drug and cosmetic colors (FD&C), drug and cosmetic colors (D&C), and external drug and cosmetic colors (Ext. D&C). A coloring agents can be used as dyes or their corresponding lakes.

In some instances, the pharmaceutical formulation can comprise a chelator. In some cases, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); 0,0′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

In some instances, a pharmaceutical formulation can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In some instances, a diluent can be used to titrate a pH of a peptide to a pH such as physiological pH to produce a salt as described above. In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In other embodiments, a pharmaceutical formulation can comprise a surfactant. Surfactants can be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, aminoacids such as L-leucine, sugar esters of fatty acids, glycerides of fatty acids or a combination thereof.

In some embodiments, a peptide can be formulated as a cleavable prodrug. The term “prodrug” as used herein, can refer to a drug precursor that, following administration to a subject and subsequent absorption, can be converted to an active, or a more active species via some process, such as conversion by a metabolic pathway. Thus, the term can encompass a derivative, which, upon administration to a recipient, can be capable of providing, either directly or indirectly, a peptide, salt or a metabolite or residue thereof. Some prodrugs can have a chemical group present on a prodrug that renders it less active and/or confers solubility or some other property to the drug. Once the chemical group has been cleaved and/or modified from the prodrug the active drug can be generated. a prodrugs can be a prodrug that can increase the bioavailability of a peptide when administered to a subject (e.g. by allowing an administered peptide to be more readily absorbed) or which enhance delivery of the peptide to a biological compartment (e.g. the brain or lymphatic system).

Also contemplated are combination products that include one or more peptides disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some instances, a pharmaceutical formulation can comprise an additional agent. In some cases, an additional agent can be present in a therapeutically effective amount in a pharmaceutical formulation.

In some instances, an additional pharmaceutical agent can be an antibiotic agent. An antibiotic agent can of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracycline. Alternatively or additionally an antibiotic agent may be effective against mycobacteria.

In some instances, an antibiotic agent may be an aminoglycoside such as Amikacin, Gentamicin, Kanamycin, Neomycin, Netilmicin, Tobramycin or Paromomycin. According to one embodiment, an antibiotic agent may be an Ansamycin such as Geldanamycin and Herbimycin

In some instances, an antibiotic agent may be a carbacephem such as Loracarbef.

In some instances, an antibiotic agent can be a carbapenem such as Ertapenem, Doripenem, Imipenem/Cilastatin or Meropenem.

In some instances, an antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively an antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole.

In some instances, an antibiotic agent may be a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some instances, an antibiotic agent may be a monobactams such as Aztreonam, or a nitrofuran such as Furazolidone or Nitrofurantoin.

In some instances, an antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some instances, an antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

In some instances, an antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some instances, an antibiotic agent may be a polypeptide such as Bacitracin, Colistin and Polymyxin B.

In some instances, an antibiotic agent may be a tetracycline such as Demeclocycline, Doxycycline, Minocycline and Oxytetracycline.

In some cases, an antibiotic agent may be effective against mycobacteria. An antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin.

In some exemplary embodiments, an antibiotic agent can include Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Methicillin, Linezolid, a Streptogramin, Tigecycline, Daptomycin, a salt of any of these, or any combination thereof.

In some instances, an additional pharmaceutical agent can be an antimicrobial agent disclosed herein. In some instances, an antimicrobial agent can be cysteamine or a salt thereof. While cysteamine can be typically used to treat conditions such as cystinosis that are not derived from an infection, the use of cysteamine as an antimicrobial compound has shown promise. For example, WO2010112848 describes the use of compositions containing cysteamine for as antimicrobial agents capable of inhibiting the formation of a bacterial biofilm for a broad range of bacterial strains, including Pseudomonas spp., Staphylococcus spp., Haemophilus spp., Burkholderia spp., Streptococcus spp., Propionibacterium spp.

In some instances, an additional pharmaceutical agent can be an antiviral agent. In some embodiments, an antiviral agent can be Acyclovir, Brivudine, Cidofovir, Docosanol, Famciclovir, Foscarnet, Fomivirsen, Ganciclovir, Idoxuridine, Penciclovir, Peramivir, Trifluridine, Valacyclovir, Vidarabine, Lamivudine, Ribavirin Amantadine, Rimantadine, a neuraminidase inhibitor, Oseltamivir, Zanamivir, a salt of any of these, or any combination thereof.

In some instances, an additional pharmaceutical agent can be an antineoplastic. In some embodiments, an antineoplastic can be selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

A weight fraction of an excipient or combination of excipients in a pharmaceutical formulation can be less than about 80%, 70%, 60%, 50%, 45%, 40%, 35%, 30%, 25%, 20%, 15%, 10%, 5%, 4%, 3%, 2%, or 1% as compared to a total weight of a pharmaceutical formulation.

A pharmaceutical formulation disclosed herein can be formulated into a variety of forms and administered by a number of different means. A pharmaceutical formulation can be administered orally, rectally, or parenterally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinyl acetate phthalate, vinylacetate crotonic acid copolymer, and ethylene-vinyl acetate copolymers; and shellac (purified lac). In some embodiments at least one polymer can function as taste-masking agents.

Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments the at least one of a fat and/or an oil can be high temperature melting. In some embodiments the at least one of a fat and/or an oil can be hydrogenated or partially hydrogenated. In some embodiments the at least one of a fat and/or an oil can be derived from a plant. In some embodiments the at least one of a fat and/or an oil can comprise at least one of glycerides, free fatty acids, and fatty acid esters. In some embodiments a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings.

Liquid formulations can include a syrup (for example, an oral formulation), an intravenous formulation, an intranasal formulation, an ocular formulation (e.g. for treating an eye infection), an otic formulation (e.g. for treating an ear infection), an ointment, a cream, an aerosol, and the like. In some instances, a combination of various formulations can be administered. In some embodiments, a tablet, pill, and the like can be formulated for an extended release profile.

In some instances, a peptide or salt thereof can be administered in a composition for topical administration. For topical administration, an active agent may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, aerosol formulations (e.g. sprays or foams), hydrogel, soaps, detergents, lotions or cakes of soap. Other conventional forms for this purpose include wound dressings, coated bandages or other polymer coverings, ointments, creams, lotions, pastes, jellies, sprays, and aerosols. Thus, a therapeutic peptide disclosed herein can be delivered via patches or bandages for dermal administration. Alternatively, a peptide can be formulated to be part of an adhesive polymer, such as polyacrylate or acrylate/vinyl acetate copolymer. For long-term applications it might be desirable to use microporous and/or breathable backing laminates, so hydration or maceration of a skin can be minimized. A backing layer can be any appropriate thickness that will provide a desired protective and support functions. A suitable thickness will generally be from about 1 to about 1000 microns. For example form about 10 to about 300 microns. Topical administration may be in the form of a nail coating or lacquer. For example, an antifungal peptide can be formulated in a solution for topical administration that contains ethyl acetate (NF), isopropyl alcohol (USP), and butyl monoester of poly[methylvinyl ether/maleic acid] in isopropyl alcohol.

Drops, such as eye drops or nose drops, may be formulated with one or more of a therapeutic peptide in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

A percentage by weight of a therapeutic agent in a composition can depend on various factors. In some cases, a therapeutic agent such as a peptide can be from about 0.01% to about 95%, from about 0.01% to about 90%, from about 0.01% to about 85%, from about 0.01% to about 80%, from about 0.01% to about 75%, from about 0.01% to about 70%, from about 0.01% to about 65%, from about 0.01% to about 60%, from about 0.01% to about 55%, from about 0.01% to about 50%, from about 0.01% to about 45%, from about 0.01% to about 40%, from about 0.01% to about 35%, from about 0.01% to about 30%, from about 0.01% to about 25%, from about 0.01% to about 20%, from about 0.01% to about 15%, from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, or from about 0.01% to about 0.1% by weight with respect to a total weight of a composition.

In some cases, a therapeutic agent such as a peptide can be at least about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3% 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 29%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight with respect to a total weight of a composition.

In some cases, a peptide can be administered in a composition with an additional agent as described herein that can be added to at least partially inhibit formation of, or destroy, a biological biofilm. In some cases, the additional agent can be from about 0.01% to about 95%, from about 0.01% to about 90%, from about 0.01% to about 85%, from about 0.01% to about 80%, from about 0.01% to about 75%, from about 0.01% to about 70%, from about 0.01% to about 65%, from about 0.01% to about 60%, from about 0.01% to about 55%, from about 0.01% to about 50%, from about 0.01% to about 45%, from about 0.01% to about 40%, from about 0.01% to about 35%, from about 0.01% to about 30%, from about 0.01% to about 25%, from about 0.01% to about 20%, from about 0.01% to about 15%, from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, or from about 0.01% to about 0.1% by weight with respect to a total weight of a composition.

In some cases, the additional agent can be at least about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3% 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 29%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight with respect to a total weight of a composition.

An aerosol can employed to administer a peptide or salt thereof to a respiratory tract. For administration by inhalation or insufflation, a composition may take the form of a dry powder, for example, a powder mix of a therapeutic agent and a suitable powder base such as lactose or starch. Therapeutic peptides can also be administered in an aqueous solution when administered in an aerosol or inhaled form. An inhalable formulation can be an inhalable respiratory formulation. Thus, other aerosol pharmaceutical formulations may comprise, for example, a physiologically acceptable buffered saline solution containing between about 0.001 mg/ml and about 100 mg/ml for example between 0, 1 and 100 mg/ml, such as 0.5-50 mg/ml, 0.5-20 mg/ml, 0.5-10 mg/ml, 0.5-5 mg/ml or 1-5 mg/ml of one or more of a peptide specific for an indication or disease to be treated.

In some instances, a formulation described herein can comprise a peptide or salt thereof as described above, with at least one of: an excipient, a diluent, or a carrier. In some instances, a pharmaceutical formulation can comprise: (a) a peptide or salt thereof comprising from about 70% to about 100% homology to a polypeptide of sequence:

(i)
(SEQ ID NO: 15)
Arg-Val-Val-Arg-Val-Val-Arg-Arg-Val-Val-Arg-Arg;
(ii)
(SEQ ID NO: 16)
Arg-Val-Val-Arg-Val-Val-Arg-Arg-Trp-Val-Arg-Arg;
(iii)
(SEQ ID NO: 17)
Arg-Trp-Trp-Arg-Trp-Trp-Arg-Arg-Trp-Trp-Arg-Arg;
(iv)
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(v)
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(vi)
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(vii)
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(viii)
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(ix)
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(x)
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(xi)
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
or
(xii)
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val;

and
(b) at least one of: an excipient, a diluent, or a carrier.

In some instances, a formulation described herein can comprise a peptide of general formula [AA₁-AA₂-AA₃-AA₄-AA₅-AA₆AA₇]ₙ. In some instances, a formulation can comprise a peptide or salt thereof of Formula A, Formula B, Formula C, Formula D, Formula E, Formula F, Formula G, Formula H, Formula I, Formula J, Formula K, Formula L, Formula M, or Formula N as described above.

In some specific embodiments, a formulation described herein can comprise a peptide of any one of SEQ ID NO:1 to SEQ ID NO:13 recited in Table 1.

A formulation described herein can comprise a peptide with from about 60% to about 70%, from about 60% to about 80%, from about 60% to about 90%, from about 60% to about 91%, from about 60% to about 95%, or from about 60% to about 100% homology to a peptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13. A formulation described herein can comprise a peptide with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% homology to a peptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

In some cases, a formulation described herein can be in unit dose form. In some instances, a peptide in a pharmaceutical formulation may not comprise 3 or more contiguous arginine or lysine residues. In some instances, a peptide or salt thereof in a pharmaceutical formulation may not be a cyclic peptide. In some instances, a peptide or salt thereof in a pharmaceutical formulation can exhibit antimicrobial activity against a bacteria with a minimum inhibitory concentration ranging from about 0.1 μg/mL to about 100 μg/mL in vitro. In some instances, a peptide or salt thereof in a pharmaceutical formulation can exhibit antiviral activity against a virus with a minimum inhibitory concentration ranging from about 0.1 μg/mL to about 100 μg/mL in vitro. In some instances, a peptide or salt thereof in a pharmaceutical formulation can exhibit antitumor activity against a tumor cell with an LD₅₀ of from about 0.01 μM to about 100 μM in vitro.

In some instances, a pharmaceutical formula can be lyophilized. In some exemplary embodiments, a pharmaceutical formulation can be stable for at least about 1 month, 2 months, 3 months, 4 months, 5 months, 6 months, 7 months, 8 months, 9 months, 10 months, 11 months, 1 year, 2 years, 3 years, 4 years, or 5 years when stored in a closed container at 10%, 20%, 30%, 40%, 50%, 60%, 70%, 80%, or 90% relative humidity at a temperature of from about 2° C. to about 30° C., from about 2° C. to about 29° C., from about 2° C. to about 28° C., from about 2° C. to about 27° C., from about 2° C. to about 26° C., from about 2° C. to about 25° C., from about 2° C. to about 24° C., from about 2° C. to about 23° C., from about 2° C. to about 22° C., from about 2° C. to about 21° C., from about 2° C. to about 20° C., from about 2° C. to about 19° C., from about 2° C. to about 18° C., from about 2° C. to about 17° C., from about 2° C. to about 16° C., from about 2° C. to about 15° C., from about 2° C. to about 14° C., from about 2° C. to about 13° C., from about 2° C. to about 12° C., from about 2° C. to about 11° C., from about 2° C. to about 10° C., from about 2° C. to about 9° C., from about 2° C. to about 8° C., from about 2° C. to about 7° C., from about 2° C. to about 6° C., from about 2° C. to about 5° C., from about 2° C. to about 4° C., or from about 2° C. to about 3° C. Stability can be determined by determined by an amount of peptide remaining after a period of time. In some instances, at least about 10%, 20%, 30%, 40%, 50%, 60%, 70%, 80%, 90%, or 100% remains after a time period. In some cases, an amount of peptide, salt, or metabolite remaining can be determined by: (a) loading a sample of a peptide or salt thereof on an HPLC equipped with a size exclusion column that is at least 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, 32, 33, 34, 35, or 36 inches in length and can comprise a silica gel; and (b) performing mass spectroscopy on at least one sample eluted from a size exclusion column. In some cases, an amount of peptide, salt, or metabolite remaining can be determined by performing an area under the curve (AUC) analysis of an HPLC chromatograph. In some cases, an amount of peptide, salt, or metabolite remaining can be determined by performing an area under the curve (AUC) analysis of a mass spectra.

Dosing/Pharmacokinetics

In some instances, a pharmaceutical formulation can be formulated to optimize pharmacokinetics/pharmacodynamics of a peptide or salt thereof contained therein.

In some cases, a peptide, salt thereof, or pharmaceutical composition comprising a peptide or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In some cases, a peptide, salt thereof, or pharmaceutical composition comprising a peptide or salt thereof described herein can be administered at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

In some cases, a peptide, salt thereof, or pharmaceutical composition comprising a peptide or salt thereof described herein can be administered to provide a blood plasma concentration of a peptide, a metabolite thereof, or salt thereof of from about 0.5 ng/mL to about 10 μg/mL, from about 1 ng/mL to about 10 μg/mL, from about 5 ng/mL to about 10 μg/mL, from about 10 ng/mL to about 10 μg/mL, from about 15 ng/mL to about 10 μg/mL, from about 20 ng/mL to about 10 μg/mL, from about 25 ng/mL to about 10 μg/mL, from about 30 ng/mL to about 10 μg/mL, from about 35 ng/mL to about 10 μg/mL, from about 40 ng/mL to about 10 μg/mL, from about 45 ng/mL to about 10 μg/mL, from about 50 ng/mL to about 10 μg/mL, from about 55 ng/mL to about 10 μg/mL, from about 60 ng/mL to about 10 μg/mL, from about 65 ng/mL to about 10 μg/mL, from about 70 ng/mL to about 10 μg/mL, from about 75 ng/mL to about 10 μg/mL, from about 80 ng/mL to about 10 μg/mL, from about 85 ng/mL to about 10 μg/mL, from about 90 ng/mL to about 10 μg/mL, from about 95 ng/mL to about 10 μg/mL, from about 100 ng/mL to about 10 μg/mL, from about 200 ng/mL to about 10 μg/mL, from about 300 ng/mL to about 10 μg/mL, from about 400 ng/mL to about 10 μg/mL, from about 500 ng/mL to about 10 μg/mL, from about 600 ng/mL to about 10 μg/mL, from about 700 ng/mL to about 10 μg/mL, from about 800 ng/mL to about 10 μg/mL, from about 900 ng/mL to about 10 μg/mL, or from about 1 μg/mL to about 10 μg/mL after a time period of from about 1 minute to about 1, 2, 3, 4, 5, 6, 7, or 10 or more hours.

In some cases a peptide, salt thereof, or pharmaceutical composition comprising a peptide or salt thereof described herein can be administered to provide a blood plasma concentration of a peptide, a metabolite thereof, or salt thereof after administration to a subject of at least about 200 ng/mL, 195 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, or 5 ng/mL after a time period of from about 1 minute to about 1, 2, 3, 4, 5, 6, 7, 8, 9, or 10 hours.

In some cases, a peptide, salt thereof, or pharmaceutical composition comprising a peptide or salt thereof described herein can be administered to provide a Tmax of a peptide, a metabolite thereof, or salt thereof after administration to a subject of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

In some cases, a peptide, salt thereof, or pharmaceutical composition comprising a peptide or salt thereof described herein can be administered to provide a Tmax of a peptide, a metabolite thereof, or salt thereof after administration to a subject of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some cases, a peptide, salt thereof, or pharmaceutical composition comprising a peptide or salt thereof described herein can be administered to provide a Tmax of a peptide, a metabolite thereof, or salt thereof after administration to a subject of at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours.

In some cases, a peptide, salt thereof, or pharmaceutical composition comprising a peptide or salt thereof described herein can be administered to provide a Cmax of a peptide, a metabolite thereof, or salt thereof after administration to a subject of at least about 1,000 μg/mL, 950 μg/mL, 900 μg/mL, 850 μg/mL, 800 μg/mL, 750 μg/mL, 700 μg/mL, 650 μg/mL, 600 μg/mL, 550 μg/mL, 500 μg/mL, 450 μg/mL, 400 μg/mL, 350 μg/mL, 300 μg/mL, 250 μg/mL, 200 μg/mL, 150 μg/mL, 100 μg/mL, or 50 μg/mL. In some cases, a peptide, salt thereof, or pharmaceutical composition comprising a peptide or salt thereof described herein can be administered to provide a Cmax of a peptide, a metabolite thereof, or salt thereof after administration to a subject of at least about 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 15 μg/mL, 10 μg/mL, 5 μg/mL, 4 μg/mL, 3 μg/mL, 2 μg/mL, or 1 μg/mL. In some cases, a peptide, salt thereof, or pharmaceutical composition comprising a peptide or salt thereof described herein can be administered to provide a Cmax of a peptide, a metabolite thereof, or salt thereof after administration to a subject of at least about 1,000 ng/mL, 950 ng/mL, 900 ng/mL, 850 ng/mL, 800 ng/mL, 750 ng/mL, 700 ng/mL, 650 ng/mL, 600 ng/mL, 550 ng/mL, 500 ng/mL, 450 ng/mL, 400 ng/mL, 350 ng/mL, 300 ng/mL, 250 ng/mL, 200 ng/mL, 150 ng/mL, 100 ng/mL, or 50 ng/mL. In some cases, a peptide, salt thereof, or pharmaceutical composition comprising a peptide or salt thereof described herein can be administered to provide a Cmax of a peptide, a metabolite thereof, or salt thereof after administration to a subject of at least about 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, or 5 ng/mL. In some cases, a peptide, salt thereof, or pharmaceutical composition comprising a peptide or salt thereof described herein can be administered to provide a Cmax of a peptide, a metabolite thereof, or salt thereof of at least about 50 ng/mL, 49 ng/mL, 48 ng/mL, 47 ng/mL, 46 ng/mL, 45 ng/mL, 44 ng/mL, 43 ng/mL, 42 ng/mL, 41 ng/mL, 40 ng/mL, 39 ng/mL, 38 ng/mL, 37 ng/mL, 36 ng/mL, 35 ng/mL, 34 ng/mL, 33 ng/mL, 32 ng/mL, 31 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 19 ng/mL, 18 ng/mL, 17 ng/mL, 16 ng/mL, 15 ng/mL, 14 ng/mL, 13 ng/mL, 12 ng/mL, 11 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or 0.5 ng/mL.

In some instances, a peptide, salt thereof, or pharmaceutical composition comprising a peptide or salt thereof described herein can be administered to provide a Cmax of a peptide, a metabolite thereof, or salt thereof of from about

In some instances, a peptide, salt thereof, or pharmaceutical composition comprising a peptide or salt thereof described herein can be administered to provide an AUC(0-t) of a peptide, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 hours after administration of a peptide, salt thereof, or pharmaceutical composition comprising a peptide or salt thereof.

In some instances, a peptide, salt thereof, or pharmaceutical composition comprising a peptide or salt thereof described herein can be administered to provide an AUC(0-t) of a peptide, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a peptide, salt thereof, or pharmaceutical composition comprising a peptide or salt thereof.

In some exemplary embodiments, a peptide, salt thereof, or pharmaceutical composition comprising a peptide or salt thereof described herein can be administered to provide an AUC(0-t) of a peptide, a metabolite thereof, or salt thereof after administration to a subject of from about 1,000 ng*h/mL to about 10,000 ng*h/mL, from about 1,000 ng*h/mL to about 9,000 ng*h/mL, from about 1,000 ng*h/mL to about 8,000 ng*h/mL, from about 1,000 ng*h/mL to about 7,000 ng*h/mL, from about 1,000 ng*h/mL to about 6,000 ng*h/mL, from about 1,000 ng*h/mL to about 5,000 ng*h/mL, from about 1,000 ng*h/mL to about 4,000 ng*h/mL, from about 1,000 ng*h/mL to about 3,000 ng*h/mL, or from about 1,000 ng*h/mL to about 2,000 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a peptide, salt thereof, or pharmaceutical composition comprising a peptide or salt thereof.

In some exemplary embodiments, a pharmaceutical formulation can be produced such that when a peptide, a salt thereof, or a pharmaceutical formulation can be administered to a primate, a peptide or salt thereof can have a Tₘₐₓ of from about 1 minute to about 1 hour, a Cₘₐₓ of from about 1 minute to about 8 hours, an AUC_{0>24 hour} of from about 0.1 μg·hr/L to about 1,000 μg·hr/L, a half-life of from about 2 hours to about 24 hours, or a combination thereof.

In some instances, a pharmaceutical formulation can be formulated such that, when a pharmaceutical formulation is administered to a subject, a peptide or salt thereof can be substantially localized in an organ of a subject. An organ can include, but is not limited to: a lung, a bladder, a gall bladder, a heart, a brain, an intestine, a stomach, an ovary, a testicle, a liver, a spleen, or a kidney.

In some instances, when a pharmaceutical formulation is administered to a subject, a peptide or salt thereof can have a half-life of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some instances, when a pharmaceutical formulation is administered to a subject, a peptide or salt thereof can have a half-life of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours. In some instances, when a pharmaceutical formulation is administered to a subject, a peptide or salt thereof can have a half-life of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

### V. Applications

A peptide disclosed herein, salt thereof, or a composition containing a peptide or salt thereof can be administered to a subject in order to at least partially ameliorate a disease or condition. A subject can be in need of a treatment of a disease or condition. In some cases, a subject may have been previously diagnosed with a disease or condition described herein, and/or may be at risk of developing a disease or condition as described herein.

As previously discussed, a peptide disclosed herein, salt thereof, or a composition containing a peptide or salt thereof can be engineered to provide a therapeutic effect by disruption of integrity of a membrane of a target. This disruption of structural integrity can occur through (a) binding to a negatively charged surface on a membrane; and/or (b) integrating into a membrane. The ability of a peptide to bind to a negatively charged surface on a membrane and/or integrate into a membrane can allow a peptide to act as a toxic agent through disruption of membrane integrity.

Use as an Antibiotic

In some cases, a peptide disclosed herein, salt thereof, or a composition containing a peptide or salt thereof can be an antibiotic. As previously described above, the production of novel antimicrobial agents, particularly antibiotics, is paramount due to the emergence of pathogens resistant to traditional antimicrobial compounds.

A peptide described herein can be engineered using a principle described herein to produce a peptide capable of (a) binding to a bacterial membrane, and/or (b) integrating into a bacterial membrane. As both gram-positive and gram-negative bacteria are known to display negatively-charged moieties on a surface of their bacterial membranes, a peptide containing positively-charged moieties can target negatively charged bacterial membranes specifically, and with broad spectrum activity. As host mammalian cell membranes can be uncharged overall due to the asymmetric expression of negatively charged moieties such as phosphatidylserine on the inner leaflet of a membrane, a peptide can be engineered to specifically target a negatively-charged bacterial membrane while avoiding an uncharged host membrane, thereby increasing the safety of a peptide. A peptide described herein can preferably target a bacterial cell. In some aspects, a peptide described herein can be toxic to a bacterial cell but not a host cell.

Furthermore, while a peptide as described herein can bind and/or integrate into a bacterial membrane of both gram-positive and gram-negative membranes, a peptide can also have the surprising and unexpected ability to bind to and block the action of lipopolysaccharides (LPS) on the surface gram-negative bacteria. LPS are large molecules consisting of a lipid and a polysaccharide composed of 0-antigen, outer core and inner core joined by a covalent bond. LPS can be found in the outer membrane of gram-negative bacteria. In some cases, LPS can elicit a strong immune response in animals. By binding to LPS molecules on the surface of a gram-negative bacterial strain, it is envisaged that the endotoxic activity of LPS can be mitigated.

In some cases, a peptide can at least partially adopt an α-helical structure. An α-helical structure can more effectively integrate into a membrane of the bacterial cell, thereby improving the ability of a peptide to disrupt the structural integrity of the bacterial membrane. In some cases, a peptide can adopt an α-helix upon synthesis. In some cases, a peptide can adopt an α-helix when in an aqueous environment. In some cases, a peptide can adopt an α-helix when contacted with a bacterial membrane.

A bacterial pathogen may be derived from a bacterial species selected from the group, but not exclusive to the group, consisting of: Staphylococcus spp., e.g. Staphylococcus aureus (e.g. Staphylococcus aureus NCTC 10442 and Staphylococcus aureus ATCC25923), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis complex; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori, Helicobacter felis; Neisseria spp., e.g. Neisseria gonorrhoea, Neisseria meningitidis; Borrelia burgdorferi; Shigella spp., e.g. Shigella flexneri; Escherichia coli (E. coli 0157:H7 NCTC 12900); Haemophilus spp., e.g. Haemophilus influenzae; Francisella tularensis; Bacillus spp., e.g. Bacillus anthraces; Clostridia spp., e.g. Clostridium botulinum, Clostridium difficile; Yersinia spp., e.g. Yersinia pestis; Treponema spp.; Burkholderia spp., e.g. Burkholderia cepacia complex, B. mallei, B pseudomallei; Propionibacterium spp., e.g. P. acnes, Acinetobacter species, an Actinomyces species, a Campylobacter species, a Candida species, Corynebacterium minutissium, Corynebacterium pseudodiphtherias, Corynebacterium stratium, Corynebacterium group G1, Corynebacterium group G2, Enterobacteriaceae, an Enterococcus species, Klebsiella pneumoniae, a Moraxella species, a non-tuberculous mycobacteria species, a Porphyromonas species, Prevotella melaninogenicus, Salmonella typhimurium, Serratia marcescens Streptococcus agalactiae, Staphylococcus salivarius, Streptococcus mitis, Streptococcus sanguis, Streptococcus pneumoniae, Vibrio cholerae, a Coccidioides species, or a Cryptococcus species.

A microbial biofilm, also referred to as a biological biofilm, can be a community of microbial cells embedded in an extracellular matrix of polymeric substances and adherent to a biological or a non-biotic surface. A range of microorganisms (bacteria, fungi, and/or protozoa, with associated bacteriophages and other viruses) can be found in these biofilms. Biofilms are ubiquitous in nature, are commonly found in a wide range of environments. Biofilms are being increasingly recognized by the scientific and medical community as being implicated in many infections, and especially their contribution to the recalcitrance of infection treatment. Biofilms can be etiologic agents for a number of disease states in mammals and are involved in 80% of infections in humans. Examples can include skin and wound infections, middle-ear infections, gastrointestinal tract infections, peritoneal membrane infections, urogenital tract infections, oral soft tissue infections, formation of dental plaque, eye infections (including contact lens contamination), endocarditis, infections in cystic fibrosis, and infections of indwelling medical devices such as joint prostheses, dental implants, catheters and cardiac implants. Microbes in biofilms can be significantly more resistant to antimicrobial treatment than their planktonic counterparts. Biofilm formation is not limited solely to the ability of microbes to attach to a surface. Microbes growing in a biofilm can interact more between each other than with the actual physical substratum on which the biofilm initially developed.

The suggested mechanisms by which biofilm-associated microorganisms elicit diseases in their host can include the following: (i) delayed penetration of the antimicrobial agent through the biofilm matrix, (ii) detachment of cells or cell aggregates from indwelling medical device biofilms, (iii) production of endotoxins, (iv) resistance to the host immune system, (v) provision of a niche for the generation of resistant organisms through horizontal gene transfer of antimicrobial resistance &/or virulence determinant genes, and (vi) altered growth rate (i.e. metabolic dormancy).

In some cases, bacteria, fungi, and/or protozoa, with associated bacteriophages and other viruses described herein can secrete a biofilm. In some cases, bacteria, fungi, and/or protozoa, with associated bacteriophages and other viruses described herein can form a biofilm. A peptide, salt thereof described herein, or a composition comprising a peptide or salt thereof described herein can be administered to at least partially penetrate, inhibit formation of, or destroy a biological biofilm. In some instances, additional agents can be added to at least partially inhibit formation of, or destroy, a biological biofilm. Non-limiting examples of additional agents can include a surfactant such as polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, sugar esters of fatty acids, and glycerides of fatty acids; a quaternary ammonium compound such as benzalkonium chloride, benzethonium chloride, methylbenzethonium chloride, cetalkonium chloride, cetylpyridinium chloride, cetrmonium, cetrimide, dofanium chloride, tetraethylammonium bromide, didecyldimethylammonium chloride and domiphen bromide; small molecules such as imidazole, indoles, nitric oxide, triazoles, phenols, sulfides, polysaccharides, furanones, and bromopyrroles; amino acids and their derivatives such as L-leucine, cysteamine, and additional peptides described herein. In some cases, an additional agent can be curcumin, apple cider vinegar, oregano, garlic, berberine, activated charcoal, or a proteolytic enzyme.

In some instances, a peptide or salt thereof described herein can have a minimum inhibitory concentration against a bacterial species described above of at least about 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/mL.

In some instances, a peptide or salt thereof described herein can have a minimum inhibitory concentration against a bacterial species described above of from about 0.001 μg/mL to about 10 μg/mL, from about 0.002 μg/mL to about 10 μg/mL, from about 0.003 μg/mL to about 10 μg/mL, from about 0.004 μg/mL to about 10 μg/mL, from about 0.005 μg/mL to about 10 μg/mL, from about 0.006 μg/mL to about 10 μg/mL, from about 0.007 μg/mL to about 10 μg/mL, from about 0.008 μg/mL to about 10 μg/mL, or from about 0.009 μg/mL to about 10 μg/mL. In some cases, a peptide or salt thereof described herein can have a minimum inhibitory concentration against a bacterial species described above of from about 0.01 μg/mL to about 1 μg/mL, from about 0.01 μg/mL to about 2 μg/mL, from about 0.01 μg/mL to about 3 μg/mL, from about 0.01 μg/mL to about 4 μg/mL, from about 0.01 μg/mL to about 5 μg/mL, from about 0.01 μg/mL to about 6 μg/mL, from about 0.01 μg/mL to about 7 μg/mL, from about 0.01 μg/mL to about 8 μg/mL, from about 0.01 μg/mL to about 9 μg/mL, or from about 0.01 μg/mL to about 10 μg/mL. In some cases, a peptide or salt thereof described herein can have a minimum inhibitory concentration against a bacterial species described above of from about 0.1 μg/mL to about 10 μg/mL, from about 0.1 μg/mL to about 10 μg/mL, from about 0.1 μg/mL to about 15 μg/mL, from about 0.1 μg/mL to about 20 μg/mL, from about 0.1 μg/mL to about 25 μg/mL, from about 0.1 μg/mL to about 30 μg/mL, from about 0.1 μg/mL to about 35 μg/mL, from about 0.1 μg/mL to about 40 μg/mL, from about 0.1 μg/mL to about 45 μg/mL, from about 0.1 μg/mL to about 50 μg/mL, from about 0.1 μg/mL to about 55 μg/mL, from about 0.1 μg/mL to about 60 μg/mL, from about 0.1 μg/mL to about 65 μg/mL, from about 0.1 μg/mL to about 70 μg/mL, from about 0.1 μg/mL to about 75 μg/mL, from about 0.1 μg/mL to about 80 μg/mL, from about 0.1 μg/mL to about 85 μg/mL, from about 0.1 μg/mL to about 90 μg/mL, from about 0.1 μg/mL to about 95 μg/mL, or from about 0.1 μg/mL to about 100 μg/mL. In some cases, a peptide or salt thereof described herein can have a minimum inhibitory concentration against a bacterial species described above of from about 0.5 μg/mL to about 10 μg/mL, from about 1 μg/mL to about 10 μg/mL, from about 1.5 μg/mL to about 10 μg/mL, from about 2 μg/mL to about 10 μg/mL, from about 2.5 μg/mL to about 10 μg/mL, from about 3 μg/mL to about 10 μg/mL, from about 3.5 μg/mL to about 10 μg/mL, from about 4 μg/mL to about 10 μg/mL, from about 4.5 μg/mL to about 10 μg/mL, from about 5 μg/mL to about 10 μg/mL, from about 5.5 μg/mL to about 10 μg/mL, from about 6 μg/mL to about 10 μg/mL, from about 6.5 μg/mL to about 10 μg/mL, from about 7 μg/mL to about 10 μg/mL, from about 7.5 μg/mL to about 10 μg/mL, from about 8 μg/mL to about 10 μg/mL, from about 8.5 μg/mL to about 10 μg/mL, from about 9 μg/mL to about 10 μg/mL, or from about 9.5 μg/mL to about 10 μg/mL. In some instances, a peptide or salt thereof described herein can have a minimum inhibitory concentration against a bacterial species described above of from about 1 μg/mL to about 1000 μg/mL, from about 1 μg/mL to about 950 μg/mL, from about 1 μg/mL to about 900 μg/mL, from about 1 μg/mL to about 850 μg/mL, from about 1 μg/mL to about 800 μg/mL, from about 1 μg/mL to about 750 μg/mL, from about 1 μg/mL to about 700 μg/mL, from about 1 μg/mL to about 650 μg/mL, from about 1 μg/mL to about 600 μg/mL, from about 1 μg/mL to about 550 μg/mL, from about 1 μg/mL to about 500 μg/mL, from about 1 μg/mL to about 450 μg/mL, from about 1 μg/mL to about 400 μg/mL, from about 1 μg/mL to about 350 μg/mL, from about 1 μg/mL to about 300 μg/mL, from about 1 μg/mL to about 250 μg/mL, from about 1 μg/mL to about 200 μg/mL, from about 1 μg/mL to about 150 μg/mL, from about 1 μg/mL to about 100 μg/mL, from about 1 μg/mL to about 95 μg/mL, from about 1 μg/mL to about 90 μg/mL, from about 1 μg/mL to about 85 μg/mL, from about 1 μg/mL to about 80 μg/mL, from about 1 μg/mL to about 75 μg/mL, from about 1 μg/mL to about 70 μg/mL, from about 1 μg/mL to about 60 μg/mL, from about 1 μg/mL to about 55 μg/mL, from about 1 μg/mL to about 50 μg/mL, from about 1 μg/mL to about 50 μg/mL, from about 1 μg/mL to about 45 μg/mL, from about 1 μg/mL to about 40 μg/mL, from about 1 μg/mL to about 35 μg/mL, from about 1 μg/mL to about 30 μg/mL, from about 1 μg/mL to about 25 μg/mL, from about 1 μg/mL to about 20 μg/mL, from about 1 μg/mL to about 15 μg/mL, from about 1 μg/mL to about 10 μg/mL, or from about 1 μg/mL to about 5 μg/mL.

In some instances, a peptide or salt thereof described herein can have a minimum inhibitory concentration described as herein against at least one of Staphylococcus aureus, methicillin resistant Staphylococcus aureus, Streptococcus pneumonia, carbapenem-resistant Enteroacteriaceae, Staphylococcus epidermidis, Staphylococcus salivarius, Corynebacterium minutissium, Corynebacterium pseudodiphtherias, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni.

A bacterial strain can also be an antibiotic-resistant variant or a bacterial strain described herein. In some cases, a bacterial strain can be resistant to an antibiotic described herein. In some instances, a bacterial strain can be resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof.

Administration of a peptide, salt thereof, or a composition comprising a peptide or salt thereof to a subject can be used to at least partially ameliorate a bacterial infection in a subject. Administration of a peptide, salt, or composition can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration of a peptide, salt, or composition can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration of a peptide, salt, or composition can be performed at least 1, 2, 3, 4, 5, 6, 7, 8, 9, 10, 11, 12, 13, 14, 15, 16, 17, 18, 19, 20, or 21 times a week. In some cases, administration of a peptide, salt, or composition can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a peptide, salt, or composition can be administered in combination with an additional antibiotic, antifungal or an antiviral agent described herein. In some exemplary embodiments, an additional antibiotic can be selected from the group consisting of: silver nitrate, Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof. In some cases, an antiviral compound can be selected from the group consisting of: Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, a salt of any of these, and any combination thereof.

In some exemplary embodiments, a peptide can be administered to a subject to treat a Staphylococcus aureus infection for a treatment duration of from about 5 days to about 30 days. Secession of treatment can be determined by an arresting of growth of a pathogen, or an amelioration of symptoms associated with an infection.

Use as an Antiviral

In some cases, a peptide disclosed herein, salt thereof, or a composition containing a peptide or salt thereof can be an antiviral agent. In some embodiments, a virus can be a DNA virus, a RNA virus, or a reverse transcriptase (retro) virus. A virus can be a dsDNA (double stranded DNA) virus, a ssDNA (single stranded DNA) virus, a dsRNA (double stranded RNA) virus, a +ssRNA (+strand or sense single stranded RNA) virus, a −ssRNA (− strand or antisense RNA) virus, a ssRNA-RT (single stranded RNA reverse transcriptase) virus, or a dsDNA-RT (double stranded DNA reverse transcriptase) virus. As described herein, a peptide described herein can be engineered to disrupt the integrity of a viral envelope of an enveloped virus. Such a disruption can at least partially reduce a viability of a virus, which can ameliorate an infection brought about by a virus.

A virus may be derived from the group, but not exclusive to the group, of a herpesvirus, a poxvirus, a hepadnavirus, a flavivirus, a togavirus, a coronavirus, hepatitis C, hepatitis D, an orthomyxovirus, a papillomavirus, a polyomaviridae, a parvovirus, a cytomegalovirus, an Epstein-Barr virus, a small pox virus, a cow pox virus, a sheep pox virus, an orf virus, a monkey pox virus, a vaccinia virus, a paramyxovirus, a retrovirus, an adenovirus, a rhabdovirus, a bunyavirus, a filovirus, an alphavirus, an arenavirus, a lentivirus, and any combination thereof. In some cases, the virus can be an enveloped virus. Examples of an enveloped viruses can include: a poxvirus, a hepadnavirus, a flavivirus, a togavirus, a coronavirus, hepatitis C, hepatitis D, an orthomyxovirus, a cytomegalovirus, an Epstein-Barr virus, a small pox virus, a cow pox virus, a sheep pox virus, an orf virus, a monkey pox virus, a vaccinia virus, a rhabdovirus, a bunyavirus, a filovirus, an alphavirus, an arenavirus, a lentivirus, and the like.

In some instances, a peptide or salt thereof described herein can have a minimum inhibitory concentration against a virus described above of at least about 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/mL.

In some instances, a peptide or salt thereof described herein can have a minimum inhibitory concentration against a virus described above of from about 0.001 μg/mL to about 10 μg/mL, from about 0.002 μg/mL to about 10 μg/mL, from about 0.003 μg/mL to about 10 μg/mL, from about 0.004 μg/mL to about 10 μg/mL, from about 0.005 μg/mL to about 10 μg/mL, from about 0.006 μg/mL to about 10 μg/mL, from about 0.007 μg/mL to about 10 μg/mL, from about 0.008 μg/mL to about 10 μg/mL, or from about 0.009 μg/mL to about 10 μg/mL. In some cases, a peptide or salt thereof described herein can have a minimum inhibitory concentration against a virus described above of from about 0.01 μg/mL to about 1 μg/mL, from about 0.01 μg/mL to about 2 μg/mL, from about 0.01 μg/mL to about 3 μg/mL, from about 0.01 μg/mL to about 4 μg/mL, from about 0.01 μg/mL to about 5 μg/mL, from about 0.01 μg/mL to about 6 μg/mL, from about 0.01 μg/mL to about 7 μg/mL, from about 0.01 μg/mL to about 8 μg/mL, from about 0.01 μg/mL to about 9 μg/mL, or from about 0.01 μg/mL to about 10 μg/mL. In some cases, a peptide or salt thereof described herein can have a minimum inhibitory concentration against a virus described above of from about 0.1 μg/mL to about 10 μg/mL, from about 0.1 μg/mL to about 10 μg/mL, from about 0.1 μg/mL to about 15 μg/mL, from about 0.1 μg/mL to about 20 μg/mL, from about 0.1 μg/mL to about 25 μg/mL, from about 0.1 μg/mL to about 30 μg/mL, from about 0.1 μg/mL to about 35 μg/mL, from about 0.1 μg/mL to about 40 μg/mL, from about 0.1 μg/mL to about 45 μg/mL, from about 0.1 μg/mL to about 50 μg/mL, from about 0.1 μg/mL to about 55 μg/mL, from about 0.1 μg/mL to about 60 μg/mL, from about 0.1 μg/mL to about 65 μg/mL, from about 0.1 μg/mL to about 70 μg/mL, from about 0.1 μg/mL to about 75 μg/mL, from about 0.1 μg/mL to about 80 μg/mL, from about 0.1 μg/mL to about 85 μg/mL, from about 0.1 μg/mL to about 90 μg/mL, from about 0.1 μg/mL to about 95 μg/mL, or from about 0.1 μg/mL to about 100 μg/mL. In some cases, a peptide or salt thereof described herein can have a minimum inhibitory concentration against a virus described above of from about 0.5 μg/mL to about 10 μg/mL, from about 1 μg/mL to about 10 μg/mL, from about 1.5 μg/mL to about 10 μg/mL, from about 2 μg/mL to about 10 μg/mL, from about 2.5 μg/mL to about 10 μg/mL, from about 3 μg/mL to about 10 μg/mL, from about 3.5 μg/mL to about 10 μg/mL, from about 4 μg/mL to about 10 μg/mL, from about 4.5 μg/mL to about 10 μg/mL, from about 5 μg/mL to about 10 μg/mL, from about 5.5 μg/mL to about 10 μg/mL, from about 6 μg/mL to about 10 μg/mL, from about 6.5 μg/mL to about 10 μg/mL, from about 7 μg/mL to about 10 μg/mL, from about 7.5 μg/mL to about 10 μg/mL, from about 8 μg/mL to about 10 μg/mL, from about 8.5 μg/mL to about 10 μg/mL, from about 9 μg/mL to about 10 μg/mL, or from about 9.5 μg/mL to about 10 μg/mL. In some instances, a peptide or salt thereof described herein can have a minimum inhibitory concentration against a virus described above of from about 1 μg/mL to about 1000 μg/mL, from about 1 μg/mL to about 950 μg/mL, from about 1 μg/mL to about 900 μg/mL, from about 1 μg/mL to about 850 μg/mL, from about 1 μg/mL to about 800 μg/mL, from about 1 μg/mL to about 750 μg/mL, from about 1 μg/mL to about 700 μg/mL, from about 1 μg/mL to about 650 μg/mL, from about 1 μg/mL to about 600 μg/mL, from about 1 μg/mL to about 550 μg/mL, from about 1 μg/mL to about 500 μg/mL, from about 1 μg/mL to about 450 μg/mL, from about 1 μg/mL to about 400 μg/mL, from about 1 μg/mL to about 350 μg/mL, from about 1 μg/mL to about 300 μg/mL, from about 1 μg/mL to about 250 μg/mL, from about 1 μg/mL to about 200 μg/mL, from about 1 μg/mL to about 150 μg/mL, from about 1 μg/mL to about 100 μg/mL, from about 1 μg/mL to about 95 μg/mL, from about 1 μg/mL to about 90 μg/mL, from about 1 μg/mL to about 85 μg/mL, from about 1 μg/mL to about 80 μg/mL, from about 1 μg/mL to about 75 μg/mL, from about 1 μg/mL to about 70 μg/mL, from about 1 μg/mL to about 60 μg/mL, from about 1 μg/mL to about 55 μg/mL, from about 1 μg/mL to about 50 μg/mL, from about 1 μg/mL to about 50 μg/mL, from about 1 μg/mL to about 45 μg/mL, from about 1 μg/mL to about 40 μg/mL, from about 1 μg/mL to about 35 μg/mL, from about 1 μg/mL to about 30 μg/mL, from about 1 μg/mL to about 25 μg/mL, from about 1 μg/mL to about 20 μg/mL, from about 1 μg/mL to about 15 μg/mL, from about 1 μg/mL to about 10 μg/mL, or from about 1 μg/mL to about 5 μg/mL.

Administration of a peptide, salt thereof, or a composition comprising a peptide or salt thereof to a subject can be used to at least partially ameliorate a viral infection in a subject. Administration of a peptide, salt, or composition can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration of a peptide, salt, or composition can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration of a peptide, salt, or composition can be performed at least 1, 2, 3, 4, 5, 6, 7, 8, 9, 10, 11, 12, 13, 14, 15, 16, 17, 18, 19, 20, or 21 times a week. In some cases, administration of a peptide, salt, or composition can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a peptide, salt, or composition can be administered in combination with an antibiotic or an additional antiviral agent disclosed herein. In some exemplary embodiments, an antibiotic agent can be selected from the group consisting of: Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof. In some cases, an additional antiviral agent can be selected from the group consisting of: Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, a salt of any of these, and any combination thereof.

Other Pathogens

Also envisaged are treatments of fungal, protozoal, or other parasitic infections by administration of a peptide described herein, salt thereof, or composition containing a peptide or salt thereof. In some cases, a pathogen can be a drug-resistant fungal, protozoal, or other parasitic organism.

A parasitic pathogen may be derived from a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthanioeba spp., Schistosoma spp., Plasmodium spp., Crytosporidium spp., Isospora spp., Balantidium spp., Loa Loa, Ascaris lumbricoides, Dirofilaria immitis, and Toxoplasma ssp., e.g. Toxoplasma gondii.

A fungal pathogen may be derived from a fungus (including yeast) selected from, but not limited to, the genera Candida spp., (e.g. C. albicans), Epidermophyton spp., Exophiala spp., Microsporum spp., Trichophyton spp., (e.g. T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremoniwn spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphiwn spp., Leptosphaeria spp., Malassezia spp. (e.g Malassezia Furfur), Mucor spp., Neotestudina spp., Nocardia spp., Nocardiopsis spp., Paecilomyces spp., Phoma spp., Piedraia spp., Pneunwcystis spp., Pseudallescheria spp., Pyrenochaeta spp., Rhizoinucor spp., Rhizopus spp., Rhodotorula spp., Saccharomyces spp., Scedosporium spp., Scopulariopsis spp., Sporobolomyces spp., S: yncephalastrum spp., Trichoderma spp., Trichosporon spp., Ulocladium spp., Ustilago spp., Verticillium spp., and Wangiella spp.

In some instances, a peptide or salt thereof described herein can have a minimum inhibitory concentration against a fungal species described above of at least about 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/mL.

In some instances, a peptide or salt thereof described herein can have a minimum inhibitory concentration against a fungal species described above of from about 0.001 μg/mL to about 10 μg/mL, from about 0.002 μg/mL to about 10 μg/mL, from about 0.003 μg/mL to about 10 μg/mL, from about 0.004 μg/mL to about 10 μg/mL, from about 0.005 μg/mL to about 10 μg/mL, from about 0.006 μg/mL to about 10 μg/mL, from about 0.007 μg/mL to about 10 μg/mL, from about 0.008 μg/mL to about 10 μg/mL, or from about 0.009 μg/mL to about 10 μg/mL. In some cases, a peptide or salt thereof described herein can have a minimum inhibitory concentration against a fungal species described above of from about 0.01 μg/mL to about 1 μg/mL, from about 0.01 μg/mL to about 2 μg/mL, from about 0.01 μg/mL to about 3 μg/mL, from about 0.01 μg/mL to about 4 μg/mL, from about 0.01 μg/mL to about 5 μg/mL, from about 0.01 μg/mL to about 6 μg/mL, from about 0.01 μg/mL to about 7 μg/mL, from about 0.01 μg/mL to about 8 μg/mL, from about 0.01 μg/mL to about 9 μg/mL, or from about 0.01 μg/mL to about 10 μg/mL. In some cases, a peptide or salt thereof described herein can have a minimum inhibitory concentration against a fungal species described above of from about 0.1 μg/mL to about 10 μg/mL, from about 0.1 μg/mL to about 10 μg/mL, from about 0.1 μg/mL to about 15 μg/mL, from about 0.1 μg/mL to about 20 μg/mL, from about 0.1 μg/mL to about 25 μg/mL, from about 0.1 μg/mL to about 30 μg/mL, from about 0.1 μg/mL to about 35 μg/mL, from about 0.1 μg/mL to about 40 μg/mL, from about 0.1 μg/mL to about 45 μg/mL, from about 0.1 μg/mL to about 50 μg/mL, from about 0.1 μg/mL to about 55 μg/mL, from about 0.1 μg/mL to about 60 μg/mL, from about 0.1 μg/mL to about 65 μg/mL, from about 0.1 μg/mL to about 70 μg/mL, from about 0.1 μg/mL to about 75 μg/mL, from about 0.1 μg/mL to about 80 μg/mL, from about 0.1 μg/mL to about 85 μg/mL, from about 0.1 μg/mL to about 90 μg/mL, from about 0.1 μg/mL to about 95 μg/mL, or from about 0.1 μg/mL to about 100 μg/mL. In some cases, a peptide or salt thereof described herein can have a minimum inhibitory concentration against a fungal species described above of from about 0.5 μg/mL to about 10 μg/mL, from about 1 μg/mL to about 10 μg/mL, from about 1.5 μg/mL to about 10 μg/mL, from about 2 μg/mL to about 10 μg/mL, from about 2.5 μg/mL to about 10 μg/mL, from about 3 μg/mL to about 10 μg/mL, from about 3.5 μg/mL to about 10 μg/mL, from about 4 μg/mL to about 10 μg/mL, from about 4.5 μg/mL to about 10 μg/mL, from about 5 μg/mL to about 10 μg/mL, from about 5.5 μg/mL to about 10 μg/mL, from about 6 μg/mL to about 10 μg/mL, from about 6.5 μg/mL to about 10 μg/mL, from about 7 μg/mL to about 10 μg/mL, from about 7.5 μg/mL to about 10 μg/mL, from about 8 μg/mL to about 10 μg/mL, from about 8.5 μg/mL to about 10 μg/mL, from about 9 μg/mL to about 10 μg/mL, or from about 9.5 μg/mL to about 10 μg/mL. In some instances, a peptide or salt thereof described herein can have a minimum inhibitory concentration against a fungal species described above of from about 1 μg/mL to about 1000 μg/mL, from about 1 μg/mL to about 950 μg/mL, from about 1 μg/mL to about 900 μg/mL, from about 1 μg/mL to about 850 μg/mL, from about 1 μg/mL to about 800 μg/mL, from about 1 μg/mL to about 750 μg/mL, from about 1 μg/mL to about 700 μg/mL, from about 1 μg/mL to about 650 μg/mL, from about 1 μg/mL to about 600 μg/mL, from about 1 μg/mL to about 550 μg/mL, from about 1 μg/mL to about 500 μg/mL, from about 1 μg/mL to about 450 μg/mL, from about 1 μg/mL to about 400 μg/mL, from about 1 μg/mL to about 350 μg/mL, from about 1 μg/mL to about 300 μg/mL, from about 1 μg/mL to about 250 μg/mL, from about 1 μg/mL to about 200 μg/mL, from about 1 μg/mL to about 150 μg/mL, from about 1 μg/mL to about 100 μg/mL, from about 1 μg/mL to about 95 μg/mL, from about 1 μg/mL to about 90 μg/mL, from about 1 μg/mL to about 85 μg/mL, from about 1 μg/mL to about 80 μg/mL, from about 1 μg/mL to about 75 μg/mL, from about 1 μg/mL to about 70 μg/mL, from about 1 μg/mL to about 60 μg/mL, from about 1 μg/mL to about 55 μg/mL, from about 1 μg/mL to about 50 μg/mL, from about 1 μg/mL to about 50 μg/mL, from about 1 μg/mL to about 45 μg/mL, from about 1 μg/mL to about 40 μg/mL, from about 1 μg/mL to about 35 μg/mL, from about 1 μg/mL to about 30 μg/mL, from about 1 μg/mL to about 25 μg/mL, from about 1 μg/mL to about 20 μg/mL, from about 1 μg/mL to about 15 μg/mL, from about 1 μg/mL to about 10 μg/mL, or from about 1 μg/mL to about 5 μg/mL.

A fungal, bacterial, or viral infection may be a systemic, topical, subcutaneous, cutaneous or mucosal infection. Topical fungal infections of nails and skin are generally caused by detinatophytes although some non-dermatophytes such as yeast can also cause skin infections. A dermatophyte infection may include a Tinea infection for example Tinea barbae (beard), Tinea capitis (head), Tinea corporis (body), Tinea cruris (groin), Tinea faciei (face), Tinea manuum (hand), Tinea pedis (foot) Tinea unguium (nail), Tinea (Pityriasis) versicolor, Tinea incognito or Tinea nigra. An infection may be derived from fungi of the genera Epidermophyton, Microsporum or Trichophyton spp. (e.g. T. rubrum and T interdigitale).

Exemplary Treatment

In some cases, a peptide, salt thereof, or composition containing a peptide or salt thereof can be administered to a subject for treatment of a dermatophytic infection. A dermatophytic infection may be an infection of a skin, lamina, stratum corneum, nails (fingernails and toenails) or hair. Of particular mention are dermatophytic infections caused by a dermatophyte of the genera Trichophyton, Epidermophyton or Microsporum. Exemplary dermatophytes can include Epidermophyton floccosum, Microsporum canis, Microsporum audouinii, Microsporum gypseum, Microsporum nanum, Microsporum ferrugineum, Microsporum distortum, Microsporum fulvum, Trichophyton rubrum, Trichophyton tnentagrophytes var. interdigitale, Trichophyton mentagrophytes var. nodulare, Trichophyton tonsurans, Trichophyton soudanese, Trichophyton violaceum, Trichophyton megnini, Trichophyton schoenlenii, Trichophyton gallinae, Trichophyton kraj denii, Trichophyton yaoundei, Trichophyton equinum, Trichophyton erinacei and Trichophyton verrucosum.

In some cases, a dermatophytic infection can be onychomycosis. The term “onychomycosis” can include, but is not limited to, distal lateral subungual, superficial white, proximal white subungual, secondary dystrophic, primary dystrophic, endonyx, candidal (e.g. onycholysis & chronic mucocutaneous disease) types of onychomycosis and Tinea ungium. Non-dermatophytic fungi associated with onychomycosis can include Aspergillus spp. Cephalosporum spp., Fusarium oxysporum, Scopularis brevicaulis, and Scytalidium spp.

A peptide described herein can be a potent antimicrobial peptides for a wide variety of pathogenic organisms. However, a peptide described herein may also be useful in a treatment of other conditions including, but not limited to, conditions associated with mucosal infections, for example, cystic fibrosis, gastrointestinal, urogenital, urinary (e.g kidney infection or cystitis) or respiratory infections.

Use as an Anti-Cancer Agent

In some cases, a peptide disclosed herein, salt thereof, or a composition containing a peptide or salt thereof can be an anticancer agent.

Rather than taking a kill all approach, as is the case for many forms of cancer treatment such as chemotherapy and radiation treatment, there is an unmet need for the production of therapeutics that specifically target cancer or tumor cells without having a toxic effect on normal, healthy cells.

One treatment strategy can be to target the vasculature of solid tumors. Destruction of the blood vessels can lead to an amplification of the anti-tumor effect, as many tumor cells rely on a single vessel for their oxygen and nutrients. Exemplary vascular targeting agents (VTAs) are described in U.S. Pat. Nos. 5,855,866, 5,965,132, 6,261,535, 6,051,230 and 6,451,312, which describe the targeted delivery of anti-cellular agents and toxins to markers of tumor vasculature.

Recently, phosphatidylserine (PS) was identified as a specific marker of tumor vasculature (Ran et al., 1998). This led to the development of new anti-PS immunoconjugates for delivering anti-cellular agents, toxins and coagulation factors to tumor blood vessels (U.S. Pat. Nos. 6,312,694, 6,783,760 and 6,818,213). As PS can be specifically overpopulated on the outer leaflet of the plasma membrane relative to normal cells, PS can be used as a marker for specific cancers. A peptide described herein can be an ideal candidate for the disruption of a structural integrity of a membrane in cancer cells in the same manner as described with respect to bacterial membranes. This can be due to the overall negative charge of the PS moiety, which can allow a peptide described herein to bind to said PS moiety and/or integrate into the membrane of the tumor cell.

A peptide described herein can be engineered to target a cancer or tumor cell, thereby alleviating cancer in a subject through at least partially arresting a growth rate of a cancer cell or tumor. In some cases, a cancer can be leukemia; melanoma; squamous cell carcinoma; neuroblastoma; colorectal adenocarcinoma; lymphoma; prostate; renal; glioblastoma; rhabdomyosarcoma; breast cancer; metastatic breast cancer; or astrocytoma. In some cases, a cancer can be a cancer that can be resistant to an existing therapeutic.

In some instances, a peptide or salt thereof described herein can exhibit antitumor activity against a tumor cell with an LD₅₀ of at least about 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/mL.

In some instances, a peptide or salt thereof described herein can exhibit antitumor activity against a tumor cell with an LD₅₀ of from about 0.001 μg/mL to about 10 μg/mL, from about 0.002 μg/mL to about 10 μg/mL, from about 0.003 μg/mL to about 10 μg/mL, from about 0.004 μg/mL to about 10 μg/mL, from about 0.005 μg/mL to about 10 μg/mL, from about 0.006 μg/mL to about 10 μg/mL, from about 0.007 μg/mL to about 10 μg/mL, from about 0.008 μg/mL to about 10 μg/mL, or from about 0.009 μg/mL to about 10 μg/mL. In some instances, a peptide or salt thereof described herein can exhibit antitumor activity against a tumor cell with an LD₅₀ of from about 0.01 μg/mL to about 1 μg/mL, from about 0.01 μg/mL to about 2 μg/mL, from about 0.01 μg/mL to about 3 μg/mL, from about 0.01 μg/mL to about 4 μg/mL, from about 0.01 μg/mL to about 5 μg/mL, from about 0.01 μg/mL to about 6 μg/mL, from about 0.01 μg/mL to about 7 μg/mL, from about 0.01 μg/mL to about 8 μg/mL, from about 0.01 μg/mL to about 9 μg/mL, or from about 0.01 μg/mL to about 10 μg/mL. In some instances, a peptide or salt thereof described herein can exhibit antitumor activity against a tumor cell with an LD₅₀ of from about 0.1 μg/mL to about 10 μg/mL, from about 0.1 μg/mL to about 10 μg/mL, from about 0.1 μg/mL to about 15 μg/mL, from about 0.1 μg/mL to about 20 μg/mL, from about 0.1 μg/mL to about 25 μg/mL, from about 0.1 μg/mL to about 30 μg/mL, from about 0.1 μg/mL to about 35 μg/mL, from about 0.1 μg/mL to about 40 μg/mL, from about 0.1 μg/mL to about 45 μg/mL, from about 0.1 μg/mL to about 50 μg/mL, from about 0.1 μg/mL to about 55 μg/mL, from about 0.1 μg/mL to about 60 μg/mL, from about 0.1 μg/mL to about 65 μg/mL, from about 0.1 μg/mL to about 70 μg/mL, from about 0.1 μg/mL to about 75 μg/mL, from about 0.1 μg/mL to about 80 μg/mL, from about 0.1 μg/mL to about 85 μg/mL, from about 0.1 μg/mL to about 90 μg/mL, from about 0.1 μg/mL to about 95 μg/mL, or from about 0.1 μg/mL to about 100 μg/mL. In some instances, a peptide or salt thereof described herein can exhibit antitumor activity against a tumor cell with an LD₅₀ of from about 0.5 μg/mL to about 10 μg/mL, from about 1 μg/mL to about 10 μg/mL, from about 1.5 μg/mL to about 10 μg/mL, from about 2 μg/mL to about 10 μg/mL, from about 2.5 μg/mL to about 10 μg/mL, from about 3 μg/mL to about 10 μg/mL, from about 3.5 μg/mL to about 10 μg/mL, from about 4 μg/mL to about 10 μg/mL, from about 4.5 μg/mL to about 10 μg/mL, from about 5 μg/mL to about 10 μg/mL, from about 5.5 μg/mL to about 10 μg/mL, from about 6 μg/mL to about 10 μg/mL, from about 6.5 μg/mL to about 10 μg/mL, from about 7 μg/mL to about 10 μg/mL, from about 7.5 μg/mL to about 10 μg/mL, from about 8 μg/mL to about 10 μg/mL, from about 8.5 μg/mL to about 10 μg/mL, from about 9 μg/mL to about 10 μg/mL, or from about 9.5 μg/mL to about 10 μg/mL. In some instances, a peptide or salt thereof described herein can exhibit antitumor activity against a tumor cell with an LD₅₀ of from about 1 μg/mL to about 1000 μg/mL, from about 1 μg/mL to about 950 μg/mL, from about 1 μg/mL to about 900 μg/mL, from about 1 μg/mL to about 850 μg/mL, from about 1 μg/mL to about 800 μg/mL, from about 1 μg/mL to about 750 μg/mL, from about 1 μg/mL to about 700 μg/mL, from about 1 μg/mL to about 650 μg/mL, from about 1 μg/mL to about 600 μg/mL, from about 1 μg/mL to about 550 μg/mL, from about 1 μg/mL to about 500 μg/mL, from about 1 μg/mL to about 450 μg/mL, from about 1 μg/mL to about 400 μg/mL, from about 1 μg/mL to about 350 μg/mL, from about 1 μg/mL to about 300 μg/mL, from about 1 μg/mL to about 250 μg/mL, from about 1 μg/mL to about 200 μg/mL, from about 1 μg/mL to about 150 μg/mL, from about 1 μg/mL to about 100 μg/mL, from about 1 μg/mL to about 95 μg/mL, from about 1 μg/mL to about 90 μg/mL, from about 1 μg/mL to about 85 μg/mL, from about 1 μg/mL to about 80 μg/mL, from about 1 μg/mL to about 75 μg/mL, from about 1 μg/mL to about 70 μg/mL, from about 1 μg/mL to about 60 μg/mL, from about 1 μg/mL to about 55 μg/mL, from about 1 μg/mL to about 50 μg/mL, from about 1 μg/mL to about 50 μg/mL, from about 1 μg/mL to about 45 μg/mL, from about 1 μg/mL to about 40 μg/mL, from about 1 μg/mL to about 35 μg/mL, from about 1 μg/mL to about 30 μg/mL, from about 1 μg/mL to about 25 μg/mL, from about 1 μg/mL to about 20 μg/mL, from about 1 μg/mL to about 15 μg/mL, from about 1 μg/mL to about 10 μg/mL, or from about 1 μg/mL to about 5 μg/mL.

Administration of a peptide, salt thereof, or a composition comprising a peptide or salt thereof to a subject can be used to at least partially ameliorate a cancer in a subject. Administration of a peptide, salt, or composition described herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 500, 1000, 1500, 2000, 3000, 5000, or 10000 consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, from about 29 to about 30 days, from about 40 to about 50 days, from about 50 to about 100 days, from about 100 to about 150 days, or from about 150 to about 300 days.

Administration of a peptide, salt, or composition thereof can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration of a peptide, salt, or composition can be performed at least 1, 2, 3, 4, 5, 6, 7, 8, 9, 10, 11, 12, 13, 14, 15, 16, 17, 18, 19, 20, or 21 times a week. In some cases, administration of a peptide, salt, or composition can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a peptide, salt, or composition can be administered in combination with an additional intervention for cancer therapy. In some cases, a peptide, salt, or composition thereof disclosed herein can be administered in combination with surgery, chemotherapy, radiation therapy, targeted therapy, immunotherapy, stem cell transplantation, hyperthermia treatment, photodynamic therapy, blood product donation and transfusion therapy, laser therapy or a combination thereof. In some embodiments, interventions can include antineoplastics, neoadjuvants, and the like. In some exemplary embodiments, an anticancer agent/compound can be selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

Clinical Trials

In some cases, a subject can be a subject in a clinical trial. A clinical trial can include a preclinical analysis, in which a subject can be screened for enrollment in a clinical trial.

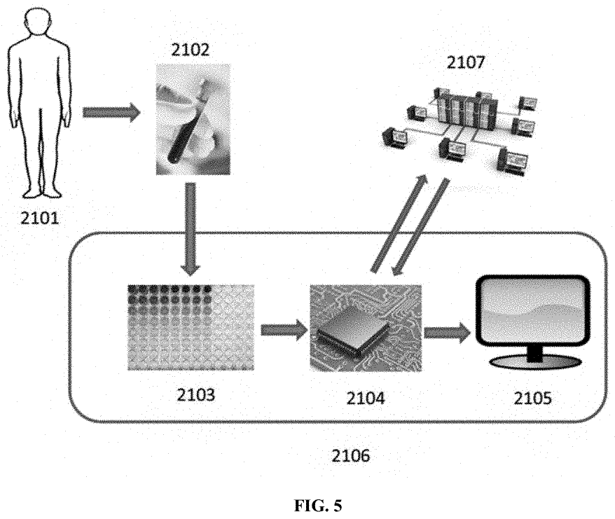

FIG. 5 depicts an exemplary an analysis of efficacy of a therapeutic in a patient from a clinical trial. After administration of a therapeutic to a patient, a patient sample 2102 such as blood can be collected from a subject 2101. An instrument 2106 can be employed to analyze a patient sample to determine the efficacy of the therapeutic. An in vitro assay 2103 can be employed to detect the presence or absence of a marker indicative of the disease or condition. For example, a level of a prostate-specific antigen (PSA) can be used to monitor a progression of prostate cancer. The results of the clinical trial can be stored locally on a storage means 2104, or a wireless storage means 2107 such as an external hard drive or cloud based storage network. The results can be displayed and analyzed on an output means 2105, which can be used, for example, by a health care professional or laboratory technician to determine the efficacy of the therapeutic in a clinical trial.

In some instances, a subject can be in a treatment facility such as a hospital, doctor's office, urgent care or outpatient clinic, or hospice. In some embodiments, a healthcare professional in a treatment facility can administer a peptide disclosed herein, salt thereof, or a composition containing a peptide or salt thereof to a subject. In some cases, a healthcare professional can diagnose a subject prior to an administration. In some cases, a healthcare professional can administer a peptide, salt thereof, or a composition containing a peptide or salt thereof to a subject as a prophylactic. A prophylactic can be administered to a subject at risk of developing a disease or condition that can be at least partially treatable by a peptide disclosed herein, salt thereof, or a composition containing a peptide or salt thereof. A subject at risk can include a subject that with a predisposition to a disease or condition as determined by an in vitro assay such as genetic testing. A subject at risk can also include a subject that can be exposed through occupation to a pathogen treatable with a peptide, salt thereof, or a composition containing a peptide or salt thereof. This can include laboratory technicians, healthcare professionals, military personnel, or law enforcement professionals.

In some cases, a subject can be in an alternative care facility. Examples of alternative care facilities can include a holistic care facility, a nursing home, or a retirement home. In some cases, a subject can be under home care. In such examples, a peptide, salt thereof, or a composition containing a peptide or salt thereof can be administered by a non-licensed healthcare professional to include a subject themselves.

Other Applications

Also disclosed herein are methods of producing a coating comprising a peptide disclosed herein, salt thereof, or a composition containing a peptide or salt thereof. A coating can be an antimicrobial coating that can be applied to a surface to remove contaminants from a surface, or to prevent contamination in the first instance. A coating can comprise an antimicrobial peptide disclosed herein. A coating can generally be prepared by contacting a coating material with a peptide disclosed herein, salt thereof, or a composition containing a peptide or salt thereof.

In some cases, a coating can be in the form of a film, sheet, liquid, or aerosol used to coat a biological or non biological surface. A film can be prepared by coating material capable of producing a film with a peptide disclosed herein, salt thereof, or a composition containing a peptide or salt thereof. A coating material capable of producing a film can be an adhesive compound, such as a mucoadhesive, used to bind a compound to a biological surface. An exemplary mucoadhesive can be a highly negatively charged polymer such as polycarbophil. A coating material capable of producing a film can be adhered to a biological surface to treat or prevent an infection on a biological surface. For example, a peptide described herein can be formulated as a coating for adherence onto an open wound, thereby eliminating a need for a bandage by directly adhering an antimicrobial compound to a site of action. Further applications can include adhering a coating onto a transplanted organ to prevent infection by a pathogen during a transplant process.

In some cases, a coating can comprise a peptide disclosed herein, salt thereof, or a composition containing a peptide or salt thereof can be used to sterilize a surface. For example, a coating can be applied to surgical equipment, and any surface in contact with surgical equipment, prior to an operation. Such practice can mitigate a risk contamination of the surgical equipment during transport. Scientific equipment can also be coated with such a coating to prevent cross contamination of certain microbes that could interfere with a measurement to be taken with the equipment.

Further examples of the use of a coating containing a peptide described herein can include coating an article such as a medical device. In some cases, the medical device can be an implantable medical device. For example, a medical device such as a catheter or prosthetic limb can be coated with a coating as described above to prevent contamination during packaging, storage, or during a transplant operation. In some cases, a peptide can be the sole antimicrobial compound in a coating. In other instances, a coating can comprise other antimicrobial compounds such as those described herein. Metallic antimicrobial compounds such as silver nitrate can also be used in combination with a peptide scribed herein.

An article for implant in contact with a coating containing a peptide, salt thereof, or pharmaceutical composition can be assembled as a composition containing an article and coating.

### VI. Kits

Disclosed herein are kits. A kit can comprise a peptide, salt thereof, formulation, or composition described herein. In some aspects, a peptide, formulation, or composition can be packaged in a container. In some aspects, a kit can further comprise instructions that direct administration of a unit dose of a peptide or formulation to a subject. In some aspects, a kit can comprise a peptide disclosed herein and instructions for the use thereof.

Methods of making a kit can include placing a peptide, salt thereof, formulation, or composition described herein in a container for packaging. A method can further comprise an inclusion of instructions for use. In some cases, instructions for use can direct administration of a unit dose of a peptide or formulation to a subject.

### VII. Examples

### Example 1: In Vitro Efficacy of Exemplary Compounds

Exemplary Peptides

Peptides were synthesized by standard peptide synthesis. Exemplary peptides screened include RRWVRRVRRVWRRVVRVVRRWVRR (SEQ ID NO:1); IRRRRRRIRRRRRR (SEQ ID NO:2); IRRRIRRIRRRIRRIRRRIRR (SEQ ID NO:3); IRRIIRRIRRIIRRIRRIIRR (SEQ ID NO:4); VWRWVRRVWRWVRRVWRWVRR (SEQ ID NO: 5); VWRWVRRVWRWVRR (SEQ ID NO:6); VVRVVRRVVRVVRR (SEQ ID NO:7); and VVRVVRVVVRVVRVVVRVVRV (SEQ ID NO:8).

Each peptide was formulated in PBS buffer prior to testing. Peptides of SEQ ID NO:5 and SEQ ID NO:8 were brought up in 100% DMSO with 0.002% polysorbate-80 at 1.28 mg/mL (40× the highest final test concentration of 32 mcg/mL). Final test concentration of DMSO was 2.5% in the assay at the first dilution for those two compounds.

Test Medium

Mueller Hinton II broth (MHB II; BD; Lot Nos. 6258541 and 7143896) was used for MIC testing of aerobic organisms. 0.002% polysorbate 80 (P80) was added to the test medium.

Broth Microdilution MIC Methodology

MIC values were determined using a broth microdilution procedure described by CLSI (1, 3). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

To prepare the drug mother plates, which would provide the serial peptide dilutions for the replicate daughter plates, the wells of columns 2-12 of standard 96-well microdilution plates (Costar 3795) were filled with 150 μl of the designated diluent for each row of peptide. The test articles and comparator compounds (300 μl at 40× the highest concentration to be tested) were dispensed into the appropriate wells in column 1. The Biomek 2000 was then used to make 2-fold serial dilutions in the mother plates from column 1 through column 11. The wells of Column 12 contained no drug and served as the organism growth control wells for the assay.

The daughter plates were loaded with 185 μL per well of MHB II using the Multidrop 384. The daughter plates were completed on the Biomek FX instrument which transferred 5 uL of peptide solution from each well of a mother plate to the corresponding well of each daughter plate in a single step. A standardized inoculum of each test organism was prepared per CLSI methods. The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. The plates were then inoculated with 10 μL of the inoculum resulting in a final cell density of approximately 5×10⁵ CFU/mL, per well.

Plates were stacked 3-4 high, covered with a lid on the top plate, placed in plastic bags, and incubated at 35° C. for 16 to 20 hrs. Following incubation, the microplates were removed from the incubator and viewed from the bottom using a plate viewer. For each date of assay, an uninoculated solubility control plate was observed for sterility and evidence of drug precipitation. The MIC was read and recorded as the lowest concentration of peptide that inhibited visible growth of the organism.

The results of the bacterial inhibition study are depicted in Table 2.

TABLE 2

| | MIC (μg/mL) | | | | | | | | | | | | |
|---|---|---|---|---|---|---|---|---|---|---|---|---|---|
| | | Formu- | Formu- | Formu- | Formu- | Formu- | Formu- | Formu- | | | | | |
| | | la A | la B | la D | la E1 | la E3 | la E4 | la G | | | | | |
| | SEQ ID | SEQ ID | SEQ ID | SEQ ID | SEQ ID | SEQ ID | SEQ ID | SEQ ID | | | | | |
| Isolate | NO: 1 | NO: 2 | NO: 3 | NO: 4 | NO: 5 | NO: 6 | NO: 7 | NO: 8 | Colistin | Levofloxacin | | Meropenem | |
| Staphylococcus | 8 | 16 | 8 | 8 | 16 | 0.5 | >32 | >32 | >32 | 0.25¹ | (0.06-0.5)² | 0.12 | (0.03-0.12) |
| aureus | | | | | | | | | | | | | |
| ATCC 29213 | | | | | | | | | | | | | |
| (MSSA; QC) | | | | | | | | | | | | | |
| Staphylococcus | 4 | 16 | 8 | 4 | 8 | 0.5 | >32 | 32 | >32 | 0.25 | | >4 | |
| aureus | | | | | | | | | | | | | |
| ATCC 43300 | | | | | | | | | | | | | |
| (MRSA) | | | | | | | | | | | | | |
| Staphylococcus | 4 | 8 | 8 | 4 | 8 | 0.5 | >32 | >32 | >32 | 8 | | >4 | |
| aureus | | | | | | | | | | | | | |
| NRS382 | | | | | | | | | | | | | |
| (USA100; | | | | | | | | | | | | | |
| HA-MRSA) | | | | | | | | | | | | | |
| Streptococcus | 16 | >32 | 32 | 16 | 32 | 8 | >32 | >32 | >32 | 1 | (0.5-2) | 0.03 | (0.03-0.25) |
| pneumoniae | | | | | | | | | | | | | |
| ATCC 49619 | | | | | | | | | | | | | |
| (QC) | | | | | | | | | | | | | |
| Streptococcus | 8 | 32 | 16 | 16 | 32 | 4 | 16 | 16 | >32 | 0.5 | | ≤0.004 | |
| pyogenes | | | | | | | | | | | | | |
| ATCC 49399 | | | | | | | | | | | | | |
| Streptococcus | 8 | >32 | 32 | 8 | 32 | 8 | >32 | 32 | >32 | 0.5 | | 0.008 | |
| agalactiae | | | | | | | | | | | | | |
| ATCC 13813 | | | | | | | | | | | | | |
| Enterococcus | 8 | >32 | 32 | 8 | 16 | 2 | >32 | 4 | >32 | 1 | (0.25-2) | 4 | (2-8) |
| faecalis | | | | | | | | | | | | | |
| ATCC 29212 | | | | | | | | | | | | | |
| (VSE; QC) | | | | | | | | | | | | | |
| Enterococcus | 4 | >32 | >32 | 8 | 16 | 4 | >32 | 4 | >32 | 0.5 | | 4 | |
| faecalis | | | | | | | | | | | | | |
| ATCC 700802 | | | | | | | | | | | | | |
| (V583 VRE) | | | | | | | | | | | | | |
| Enterococcus | 2 | 16 | 4 | 4 | 8 | 0.5 | 16 | 0.25 | >32 | 16 ³ | | >4 | |
| faecium | | | | | | | | | | | | | |
| ATCC 51559 | | | | | | | | | | | | | |
| (VRE) | | | | | | | | | | | | | |
| Escherichia | 4 | 16 | 4 | 4 | 16 | 2 | >32 | >32 | 0.12 (0.25-2) | 0.015 | (0.008-0.06) | 0.015 | (0.008-0.06) |
| coli | | | | | | | | | | | | | |
| ATCC 25922 | | | | | | | | | | | | | |
| (QC) | | | | | | | | | | | | | |
| Escherichia | 8 | 16 | 4 | 8 | 16 | 1 | 32 | >32 | 0.12 | 0.03 | | 0.015 | |
| coli | | | | | | | | | | | | | |
| ATCC 35218 | | | | | | | | | | | | | |
| (ESBL) | | | | | | | | | | | | | |
| Escherichia | 2 | 32 | 4 | 4 | 32 | 1 | 16 | >32 | 0.06 | 64 | | >4 | |
| coli | | | | | | | | | | | | | |
| ATCC | | | | | | | | | | | | | |
| BAA-2471 | | | | | | | | | | | | | |
| (NDM-1) | | | | | | | | | | | | | |
| Klebsiella | 8 | >32 | 32 | 32 | >32 | 4 | >32 | >32 | 0.12 | 64 | | >4 | |
| pneumoniae | | | | | | | | | | | | | |
| ATCC | | | | | | | | | | | | | |
| BAA-1705 | | | | | | | | | | | | | |
| (KPC) | | | | | | | | | | | | | |
| Pseudomonas | 8 | >32 | 4 | 8 | >32 | 16 | >32 | >32 | 0.25 (0.5-4) | 1 | (0.5-4) | 0.25 | (0.12-1) |
| aeruginosa | | | | | | | | | | | | | |
| ATCC 27853 | | | | | | | | | | | | | |
| (non-MDR; | | | | | | | | | | | | | |
| QC) | | | | | | | | | | | | | |

| | | | | | | | | | | | |
|---|---|---|---|---|---|---|---|---|---|---|---|
| Pseudomonas | 4 | 8 | 2 | 4 | 16 | 8 | >32 | 16 | 0.12 | 1 | 0.015 |
| aeruginosa | | | | | | | | | | | |
| ATCC | | | | | | | | | | | |
| BAA-2109 | | | | | | | | | | | |
| (MDR) | | | | | | | | | | | |
| Acinetobacter | 2 | 32 | 8 | 8 | 16 | 0.5 | >32 | 32 | 0.25 | 0.25 | 0.5 |
| baumannii | | | | | | | | | | | |
| ATCC 19606 | | | | | | | | | | | |
| (non-MDR) | | | | | | | | | | | |
| Acinetobacter | 4 | >32 | 16 | 8 | 32 | 0.5 | >32 | >32 | 0.5 | 4 | >4 |
| baumannii | | | | | | | | | | | |
| ATCC | | | | | | | | | | | |
| BAA-1797 | | | | | | | | | | | |
| (MDR) | | | | | | | | | | | |

Peptides described herein displayed superior antimicrobial activity as determined by MIC against the bacteria species tested when compared to traditional antibiotics Colistin, Levofloxacin, and Meropenem.

### Example 2: Exemplary In Vitro Data for SEQ ID NO:1

Test Compounds and Comparators

An exemplary peptide of SEQ ID NO:1 was employed for this study. SEQ ID NO:1 was stored at −20° C. prior to testing. A stock solution of SEQ ID NO:1 was prepared at 40× the final testing concentration, aliquoted and stored at −20° C. prior to testing. Comparator antibiotics were supplied by Micromyx, and stock solutions were prepared on the first day of testing using solvents recommended by CLSI (1). Stock solutions of all compounds were made at 40× the final testing concentration and stored at −80° C. prior to testing. Information regarding compound source, catalog and lot number, testing concentrations and drug diluent for the comparators and test agent are detailed below in Table 3.

| Test agent | Supplier | Cat. No. | Lot number | Testing (μg/mL) | Solvent/Diluent |
|---|---|---|---|---|---|
| SEQ ID NO: 1 | N/A | N/A | N/A | 128-0.12 | PBS-0.002% P80/ |
| | | | | | PBS-0.002% P80 |
| Amikacin | Sigma | A2324 | 036K1588 | 64-0.06 | Water/Water |
| Ceftolozane | Remal | — | M17223A, M17424 | 32-0.03 | NA/Water |
| Piperacillin | Sigma | P8396 | SLBD9664V | 128-0.12 | Water/Water |
| Tazobactam | USP | 1643383 | HOM200 | 4 | Water/Water |
| Meropenem | USP | 1392454 | J0K434 | 8-0.008 | Water/Water |
| Tobramycin | Sigma | T1783 | 109K1184 | 64-0.06 | Water/Water |
| Ceftazidime | Sigma | C3809 | 076M4770V | 32-0.03 | Water/Water |
| Colistin | Sigma | C4461 | SLBN5158V | 32-0.03 | Water/Water |
| Levofloxacin | Sigma | 28266 | BCBC2112V | 8-0.008 | Water and 0.1M |
| | | | | | NaOH/Water |
| Linezolid | ChemPacific | 35710 | CHPC091007-01 | 64-0.06 | Water/Water |
| | | | | 32-0.03 | |
| Daptomycin | Cubist | — | MCB2009 | 32-0.03 | Water/Water |
| Vancomycin | Sigma | V2002 | 080M1341V | 32-0.03 | Water/Water |
| Doxycycline | Sigma | D9891 | BCBF9827V | 32-0.03 | Water/Water |
| Trimethoprim | Sigma | T0667 | 082M4076V | 8-0.008 | Water/Water |
| Sulfamethoxazole | Fluka | S7507 | BCBC7096V | 144-0.14 | Hot water and 2.5M |
| | | | | | NaOH/Water |
| Clindamycin | Sigma | C5269 | 021M1533 | 16-0.015 | Water/Water |

Test Organisms

The test organisms evaluated in this study consisted of clinical isolates from the Micromyx Repository and reference isolates from the American Type Culture Collection (ATCC; Manassas, Va.). The clinical isolates were obtained from USA hospitals. Upon initial receipt at Micromyx, the organisms were sub-cultured onto an appropriate agar medium. Following incubation, colonies were harvested from these plates and cell suspensions prepared and frozen at −80° C. with a cryoprotectant. Prior to testing, the isolates were streaked from frozen vials onto Trypticase Soy Agar with 5% sheep blood (Becton Dickenson [BD]; Sparks, Md.; Lot Nos. 7173618, 7166759, 7208688, 72144895, 7228505 and Remel Lenexa, Kans. Lot No. 212574). Plates were incubated at 35° C. overnight.

Test Medium

Mueller Hinton II broth (MHB II; BD; Lot Nos. 6258541 and 7143896) was used for MIC testing of aerobic organisms. For SEQ ID NO:1 0.002% polysorbate 80 (P80) was added to the test medium.

Broth Microdilution MIC Methodology

MIC values were determined using a broth microdilution procedure described by CLSI (1, 3). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

To prepare the drug mother plates, which would provide the serial drug dilutions for the replicate daughter plates, the wells of columns 2-12 of standard 96-well microdilution plates (Costar 3795) were filled with 150 μl of the designated diluent for each row of drug. The test articles and comparator compounds (300 μl at 40× the highest concentration to be tested) were dispensed into the appropriate wells in column 1. The Biomek 2000 was then used to make 2-fold serial dilutions in the mother plates from column 1 through column 11. The wells of Column 12 contained no drug and served as the organism growth control wells for the assay.

The daughter plates were loaded with 185 μL per well of MHB II using the Multidrop 384. The daughter plates were completed on the Biomek FX instrument which transferred 5 □L of drug solution from each well of a mother plate to the corresponding well of each daughter plate in a single step. A standardized inoculum of each test organism was prepared per CLSI methods (1). The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. The plates were then inoculated with 10 μL of the inoculum resulting in a final cell density of approximately 5×105 CFU/mL, per well.

Plates were stacked 3-4 high, covered with a lid on the top plate, placed in plastic bags, and incubated at 35° C. for 16 to 20 hrs. Following incubation, the microplates were removed from the incubator and viewed from the bottom using a plate viewer. For each date of assay, an uninoculated solubility control plate was observed for sterility and evidence of drug precipitation. The MIC was read and recorded as the lowest concentration of drug that inhibited visible growth of the organism.

Results and Discussion

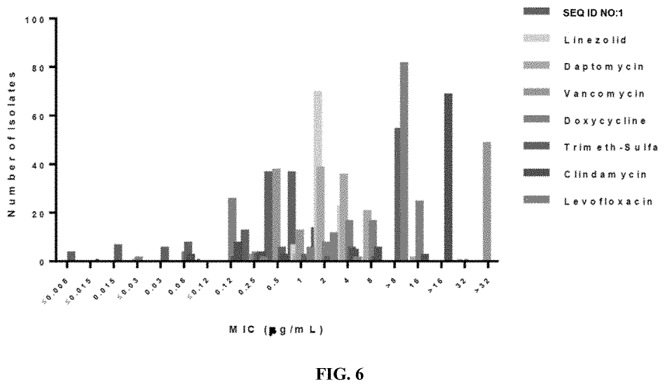

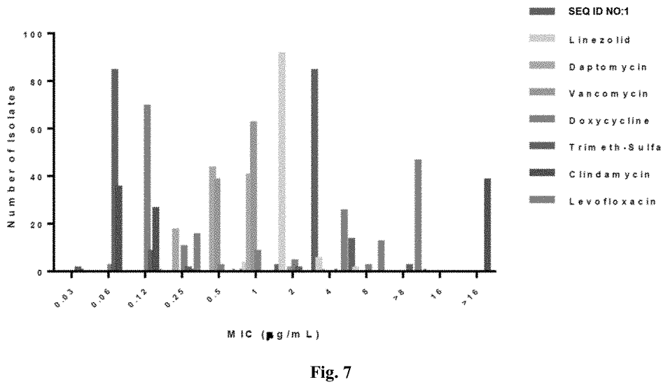

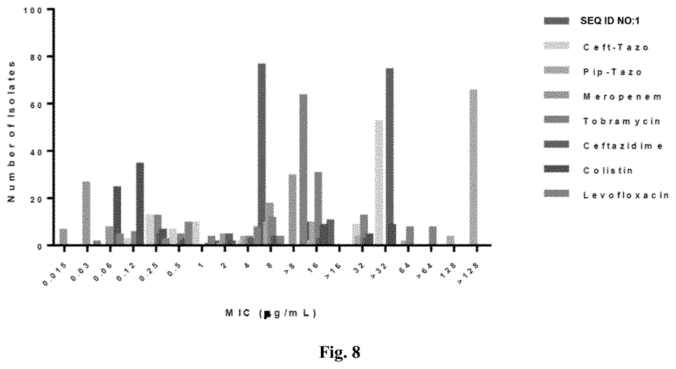

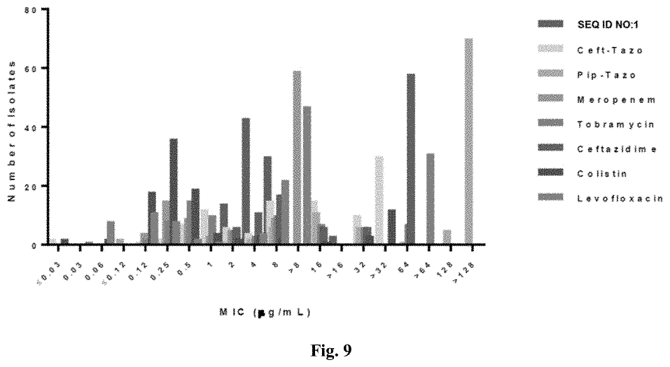

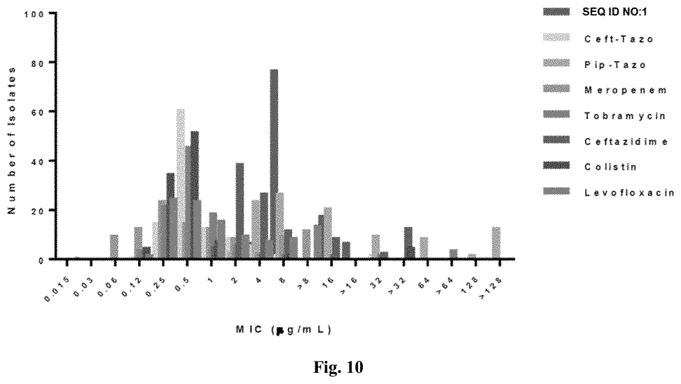

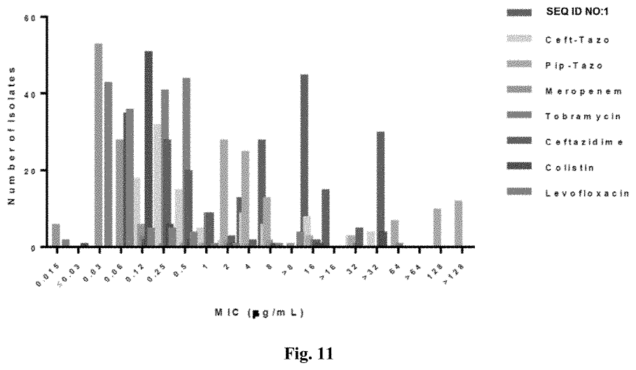

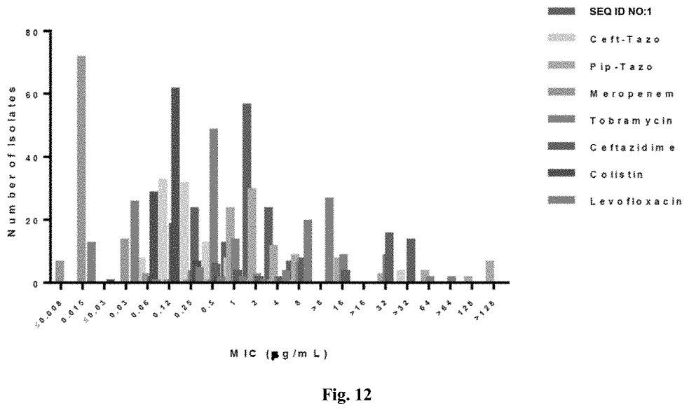

The summary data for evaluating SEQ ID NO:1 against the ESKAPE pathogens and E. coli are shown in Tables 4-10 and FIGS. 6-12. The tables contain the MIC ranges, modes, MIC50 and MIC90 values, whereas the figures show the MIC distributions, for SEQ ID NO:1 and each comparator against Enterococcus faecium (Table 4 and FIG. 6), Staphylococcus aureus (Table 5 and FIG. 7), Klebsiella pneumoniae (Table 6 and FIG. 8), Acinetobacter (Table 7 and FIG. 9), Pseudomonas aeruginosa (Table 8 and FIG. 10), Enterobacter (Table 9 and FIG. 11) and Escherichia coli (Table 10 and FIG. 12). The MIC values for the control drugs against the QC organisms were within established CLSI QC ranges, with the exception of colistin on two test days. Clinical breakpoints to determine percent resistance within a given organism group were obtained from CLSI (3), with the exception of colistin for K. pneumoniae, Enterobacter spp., and E. coli, which are only available from EUCAST.

Significant precipitation was observed during broth dilution testing of SEQ ID NO:1 at the concentrations of 128, 64 and 32 μg/mL, and slight precipitation was observed at 16 and 8 μg/mL. Depending on the organism and test day, MICs could be read occasionally with either of these two concentrations.

As shown in Table 4, against E. faecium (n=104) SEQ ID NO:1 had an MIC50/90 value of 1/2 μg/mL, with a range of <0.12 to 4 μg/mL. Nearly half of these isolates were vancomycin-resistant and four were linezolid-resistant. In addition, 25% were resistant to doxycycline and 78.8% were levofloxacin-resistant. SEQ ID NO:1 was the most active agent against, followed by linezolid with MIC50/90 values of 2/4 μg/mL and an MIC range of 1 to 32 μg/mL; vancomycin was the least active with MIC50/90 values of 1/>32 and an MIC range of 0.25 to >32 μg/mL. FIG. 6 shows the MIC distributions of SEQ ID NO:1 and the comparator drugs against the E. faecium isolates, with SEQ ID NO:1 displaying a fairly narrow distribution against the majority of isolates

TABLE 4

| | Drug | MIC range | Mode | MIC₅₀ | MIC₉₀ | % R¹ |
|---|---|---|---|---|---|---|
| Enterococcus | SEQ ID NO: 1 | ≤0.12-4 | 1 | 1 | 2 | — |
| faecium | Linezolid | 1-32 | 2 | 2 | 4 | 3.8 |
| (n = 104) | Daptomycin | 0.06->32 | 2 | 4 | 8 | — |
| | Vancomycin | 0.25->32 | >32 | 1 | >32 | 47.1 |
| | Doxycycline | ≤0.03-32 | 0.12 | 4 | 16 | 25 |
| | Trimethoprim - | ≤0.008->8 | >8 | >8 | >8 | — |
| | Sulfamethoxazole | | | | | |
| | Clindamycin | ≤0.15->16 | >16 | >16 | >16 | — |
| | Levofloxacin | 0.25->8 | >8 | >8 | >8 | 78.8 |

Against the collection of S. aureus isolates (Table 5; n=104), SEQ ID NO:1 had an MIC range of 1-16 μg/mL with an MIC50 of 4 μg/mL and an MIC90 of 8 μg/mL. All of these isolates were methicillin-resistant; there were two that were also linezolid-resistant. Over 80% were resistant to levofloxacin and 37.5% of these S. aureus isolates were resistant to clindamycin. Trimethoprim-sulfamethoxazole was the most active agent against this set of organisms, with MIC50/90 values of 0.06/0.12 μg/mL and an MIC range of 0.03 to >8 μg/mL. Levofloxacin and clindamycin were the least active agents against this set of S. aureus (MIC50/90=8/>8 and 0.12/>16 μg/mL, respectively). The MIC distributions against the S. aureus isolates are shown in FIG. 7, with SEQ ID NO:1 demonstrating a narrow distribution, mostly between 2 and 8 μg/mL.

TABLE 5

| | Drug | MIC range | Mode | MIC₅₀ | MIC₉₀ | % R¹ |
|---|---|---|---|---|---|---|
| Staphylococcus | SEQ ID NO: 1 | 1-16 | 4 | 4 | 8 | — |
| aureus | Linezolid | 1-8 | 2 | 2 | 2 | 1.9 |
| (n = 104) | Daptomycin | 0.25-4 | 0.5 | 0.5 | 1 | — |
| | Vancomycin | 0.5-2 | 1 | 1 | 1 | 0 |
| | Doxycycline | 0.06-8 | 0.12 | 0.12 | 1 | 0 |
| | Trimethoprim - | 0.03->8 | 0.06 | 0.06 | 0.12 | 3.8 |
| | Sulfamethoxazole | | | | | |
| | Clindamycin | 0.03->16 | >16 | 0.12 | >16 | 37.5 |
| | Levofloxacin | 0.12->8 | >8 | 8 | >8 | 82.7 |

Table 6 shows that SEQ ID NO:1 had an MIC range of 2 to >16 μg/mL and MIC50/90 of 8/>16 μg/mL against the panel of K. pneumoniae (n=101). As shown in the MIC distribution of FIG. 8, SEQ ID NO:1 had an MIC of 8 μg/mL against the majority of isolates. Over 80% of these isolates were resistant to ceftazidime, and over 60% were resistant to piperacillin-tazobactam, ceftolozane-tazobactam, tobramycin, or levofloxacin. Fifty percent were meropenem-resistant, whereas 27.7% were colistin-resistant. Meropenem and colistin had the best activity against this set of K. pneumoniae, though FIG. 8 shows that both drugs had a bimodal distribution due to the presence of resistant organisms within the set, including those producing extended-spectrum β-lactamases (ESBLs) or that were colistin-resistant. The comparator with the weakest activity by MIC50/90 was piperacillin-tazobactam (>128/>128 μg/mL).

TABLE 6

| | Drug | MIC range | Mode | MIC₅₀ | MIC₉₀ | % R¹ |
|---|---|---|---|---|---|---|
| Klebsiella | SEQ ID NO: 1 | 2->16 | 8 | 8 | >16 | — |
| pneumoniae | Ceftolozane- | 0.12->32 | >32 | >32 | >32 | 63.4 |
| (n = 101) | Tazobactam | | | | | |
| | Piperacillin - | 2->128 | >128 | >128 | >128 | 69.3 |
| | Tazobactam | | | | | |
| | Meropenem | 0.015->8 | >8 | 4 | >8 | 50.5 |
| | Tobramycin | 0.12->64 | 16 | 16 | 64 | 59.4 |
| | Ceftazidime | 0.25->32 | >32 | >32 | >32 | 80.2 |
| | Colistin | 0.06->32 | 0.12 | 0.12 | 32 | 27.7 |
| | Levofloxacin | 0.03->8 | >8 | >8 | >8 | 67.3 |

The 104 Acinetobacter isolates in this study were predominantly A. baumannii (80%), but also included A. pittii, A. radioresistens, A. lwoffii and A. junii. Levofloxacin- and ceftazidime-resistance among this set were 66.3 and 61.5%, respectively, and there were 65.4% that were meropenem-resistant. Colistin-resistance (20.2%) was fairly common as well, although this drug displayed good activity (MIC50/90 of 0.25/>32 μg/mL). Against this set of Acinetobacter (Table 7), SEQ ID NO:1 demonstrated an MIC50 of 4 μg/mL and an MIC90 of 16 μg/mL, with an MIC range of 0.5 to >16 μg/mL. By MIC50/90, piperacillin-tazobactam was the comparator with the weakest activity (MIC50/90=>128/>128 μg/mL); indeed, 72.1% of the isolates in this set were resistant to this drug combination. The MIC distributions for SEQ ID NO:1 and the comparator drugs against the Acinetobacter isolates are shown in FIG. 9.

TABLE 7

| | Drug | MIC range | Mode | MIC₅₀ | MIC₉₀ | % R¹ |
|---|---|---|---|---|---|---|
| Acinetobacter | SEQ ID NO: 1 | 0.5->16 | 4 | 4 | 16 | — |
| spp. (n = 104) | Ceftolozane- | ≤0.03->32 | >32 | 16 | >32 | — |
| | Tazobactam | | | | | |
| | Piperacillin - | ≤0.12->128 | >128 | >128 | >128 | 72.1 |
| | Tazobactam | | | | | |
| | Meropenem | 0.12->8 | >8 | >8 | >8 | 65.4 |
| | Tobramycin | 0.12->64 | >64 | 8 | >64 | 49.0 |
| | Ceftazidime | 2->32 | >32 | >32 | >32 | 61.5 |
| | Colistin | ≤0.03->32 | 0.25 | 0.25 | >32 | 20.2 |
| | Levofloxacin | 0.03->8 | >8 | 8 | >8 | 66.3 |

Against the 109 P. aeruginosa isolates evaluated in this study, SEQ ID NO:1 had an MIC range of 4->16 μg/mL (MIC50/90 of 8/16 μg/mL) (Table 8). Twenty-one percent of these isolates were resistant to levofloxacin and 20% were meropenem-resistant. Resistance to colistin was observed among 7.3% of the isolates in this set. Piperacillin-tazobactam had the weakest activity by MIC50/90 (16/>128 μg/mL) though only 13.8% of isolates were resistant to the combination; the best activity against these P. aeruginosa isolates was demonstrated by colistin, with an MIC50/90 value of 0.5/1 μg/mL. As shown in the MIC distributions in FIG. 10, SEQ ID NO:1 had an MIC value of 8 μg/mL against most of the P. aeruginosa isolates, with a narrow MIC range.

TABLE 8

| | Drug | MIC range | Mode | MIC₅₀ | MIC₉₀ | % R¹ |
|---|---|---|---|---|---|---|
| Pseudomonas | SEQ ID NO: 1 | 4->16 | 8 | 8 | 16 | — |
| aeruginosa | Ceftolozane - | 0.12-32 | 0.5 | 0.5 | 2 | 1.7 |
| (n = 109) | Tazobactam | | | | | |
| | Piperacillin - | 2->128 | 8 | 16 | >128 | 13.8 |
| | Tazobactam | | | | | |
| | Meropenem | 0.06->8 | 0.25 | 0.5 | >8 | 20.2 |
| | Tobramycin | 0.12->64 | 0.5 | 0.5 | 4 | 7.4 |
| | Ceftazidime | 0.5->32 | 2 | 4 | >32 | 14.6 |
| | Colistin | 0.12->32 | 0.5 | 0.5 | 1 | 7.3 |
| | Levofloxacin | 0.015->8 | 0.25 | 1 | >8 | 21.1 |

Table 9 shows the results of evaluating SEQ ID NO:1 and comparator agents against Enterobacter aerogenes and E. cloacae isolates (n=102). Resistance to ceftazidime, piperacillin-tazobactam or ceftolozane-tazobactam was observed among 36.3, 21.5 and 19.6% of the isolates in this group. SEQ ID NO:1 had an MIC range of 2 to >16 μg/mL, with MIC50/90 values of 16 and >16 μg/mL, respectively. Meropenem had the best activity against this group, with an MIC50/90 value of 0.03/0.12 μg/mL (MIC range of 0.015->8 μg/mL). Piperacillin-tazobactam appeared to be the least active comparator agent by MIC50/90 against this set, with a bimodal MIC distribution, as shown in FIG. 11.

TABLE 9

| | Drug | MIC range | Mode | MIC₅₀ | MIC₉₀ | % R¹ |
|---|---|---|---|---|---|---|
| Enterobacter | SEQ ID NO: 1 | 2->16 | 16 | 16 | >16 | — |
| spp. (n = 102) | Ceftolozane - | 0.12->32 | 0.25 | 0.5 | 16 | 19.6 |
| | Tazobactam | | | | | |
| | Piperacillin - | 1->128 | 2 | 4 | >128 | 21.5 |
| | Tazobactam | | | | | |
| | Meropenem | 0.015->8 | 0.03 | 0.03 | 0.12 | 3.9 |
| | Tobramycin | 0.12-64 | 0.5 | 0.5 | 1 | 3.9 |
| | Ceftazidime | 0.12->32 | >32 | 1 | >32 | 36.3 |
| | Colistin | ≤0.03->32 | 0.12 | 0.12 | 0.25 | 5.8 |
| | Levofloxacin | 0.015->8 | 0.03 | 0.06 | 0.5 | 4.9 |

When evaluated against the set of E. coli isolates (n=101; Table 10), SEQ ID NO:1 demonstrated an MIC range of 1 to 8 μg/mL, and an MIC50/90 value of 2/4 μg/mL. Approximately half of the isolates in this set were ESBL-producing. Nearly 50% were resistant to levofloxacin, 33.6% were ceftazidime-resistant, and 21.7% were tobramycin-resistant. Meropenem had the best overall activity against these isolates, with MIC50/90 values of 0.015/0.03 μg/mL (MIC range of <0.008-4 μg/mL), and piperacillin-tazobactam had the weakest activity against this set based on MIC50/90 (2/64 μg/mL), although only 8.9% of isolates were resistant to the combination. The MIC distributions of SEQ ID NO:1 and the comparators are shown in FIG. 12. For SEQ ID NO:1, the majority of the results fall within a narrow range, in contrast to those of the comparators.

TABLE 10

| | Drug | MIC range | Mode | MIC₅₀ | MIC₉₀ | % R¹ |
|---|---|---|---|---|---|---|
| Escherichia | SEQ ID NO: 1 | 1-8 | 2 | 2 | 4 | — |
| coli | Ceftolozane - | 0.06->32 | 0.12 | 0.25 | 1 | 4.9 |
| (n = 101) | Tazobactam | | | | | |
| | Piperacillin - | 0.25->128 | 2 | 2 | 64 | 8.9 |
| | Tazobactam | | | | | |
| | Meropenem | ≤0.008-4 | 0.015 | 0.015 | 0.03 | 1 |
| | Tobramycin | 0.25->64 | 0.5 | 0.5 | 32 | 21.7 |
| | Ceftazidime | 0.06->32 | 0.25 | 0.5 | >32 | 33.6 |
| | Colistin | ≤0.03-0.5 | 0.12 | 0.12 | 0.12 | 0 |
| | Levofloxacin | 0.015->8 | >8 | 4 | >8 | 46.5 |

Table 11 summarizes the activity of SEQ ID NO:1 against the various resistance phenotypes in this study. Fifty of the E. faecium isolates were vancomycin-resistant; against these, SEQ ID NO:1 demonstrated MIC50/90 values of 0.5/1 μg/mL and a range of 0.25-2 μg/mL. All the S. aureus isolates in this study were MRSA and as mentioned above, SEQ ID NO:1 had an MIC range against these of 1-16 μg/mL; the MIC50/90 was 4/8 μg/mL. When the K. pneumoniae isolates in this study were parsed into colistin-resistant (n=28), ESBL (n=41) and KPC (n=43) isolates, SEQ ID NO:1 had MIC ranges of 4-16, 2-16 and 2-16 μg/mL, respectively. The MIC50/90 of SEQ ID NO:1 against the colistin-resistant, ESBL and KPC K. pneumoniae isolates were 8/16, 8/16 and 8/>8 μg/mL, respectively. Out of the 104 Acinetobacter isolates, 68 were meropenem-resistant and 21 were colistin-resistant; SEQ ID NO:1 had MIC50/90 values of 4/16 and 8/16 μg/mL and ranges of 2-16 and 4-16 μg/mL against these, respectively. Twenty-two of the P. aeruginosa isolates were meropenem-resistant, and 8 were colistin-resistant; SEQ ID NO:1 had MIC50/90 values of 8/16 μg/mL and MIC ranges of 4-16 μg/mL against both sets of resistant isolates. There were 37 ceftazidime-resistant isolates among the Enterobacter isolates; SEQ ID NO:1 demonstrated an MIC50/90 of 8/16 μg/mL and a range of 2->32 μg/ml against these. The set of E. coli isolates contained 48 ESBL strains; SEQ ID NO:1 had an MIC range of 1-8 μg/mL with an MIC50 of 2 μg/mL and an MIC90 of 4 μg/mL against these resistant organisms.

TABLE 11

| | | n | MIC₅₀ | MIC₉₀ | MIC range |
|---|---|---|---|---|---|
| E. faecium | VRE | 50 | 0.5 | 1 | 0.25-2 |
| S. aureus | MRSA | 104 | 4 | 8 | 1-16 |
| K. pneumoniae | COL^{R} | 28 | 8 | 16 | 4-16 |
| | ESBL | 41 | 8 | 16 | 2-16 |
| | KPC | 43 | 8 | >8 | 2-16 |
| Acinetobacter | MER^{R} | 68 | 4 | 16 | 2-16 |
| | COL^{R} | 21 | 8 | 16 | 4-16 |
| P. aeruginosa | MER^{R} | 22 | 8 | 16 | 4-16 |
| | COL^{R} | 8 | 8 | 16 | 4-16 |
| Enterobacter | Ceftazidime^{R} | 37 | 8 | 16 | 2->32 |
| E. coli | ESBL | 48 | 2 | 4 | 1-8 |

In summary, SEQ ID NO:1 was evaluated against panels of at least 100 of each of the ESKAPE pathogens, including those with a variety of known resistance phenotypes. Overall, SEQ ID NO:1 had a fairly narrow MIC range against each set of pathogens, irrespective of the resistance profiles contained therein. The best activity observed for SEQ ID NO:1 was against E. faecium (MIC50/90 of 1/2 μg/mL), Acinetobacter (MIC50/90 of 2/4 μg/mL), and E. coli (MIC50/90 of 2/4 μg/mL).

### Example 3: Disruption of Biofilms

P. aeruginosa or S. aureus cells were grown in vinyl microtiter plates in Mueller-Hinton broth for 24 h at 37° C. to allow a mature biofilm to form. After about 24 hrs, an exemplary peptide of SEQ ID NO:1 diluted 2-fold in MHB was added to the biofilm containing wells and incubated for 1 hour. The well-associated biomass was stained with crystal violet and quantified by measuring absorbance at 550 nm following solubilization in 30% acetic acid.

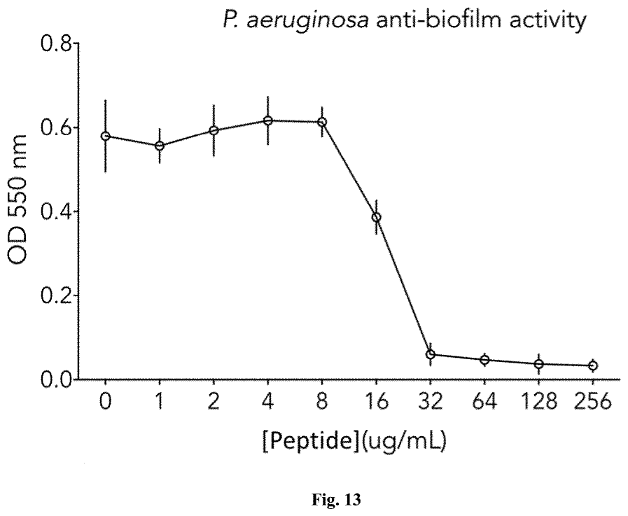

FIG. 13 shows the effect on biofilm produced by P. aeruginosa by contacting the biofilm with the exemplary peptide of SEQ ID NO:1, as determined by the absorbance at 550 nm. The exemplary peptide had a profound effect on the biofilm as a function of the concentration of the peptide.

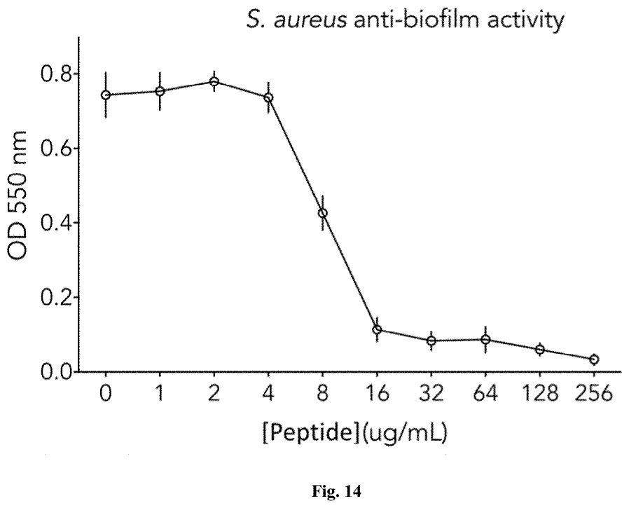

FIG. 14 shows the effect on biofilm produced by S. aureus by contacting the biofilm with the exemplary peptide of SEQ ID NO:1, as determined by the absorbance at 550 nm. The exemplary peptide had a profound effect on the biofilm as a function of the concentration of the peptide and was able to disrupt the biofilm at a lower concentration than for P. aeruginosa.

### Example 4: Chemical Peptide Synthesis of Arg-Ser-Arg-Val-Val-Arg-Ser-Trp-Ser-Arg-Val (SEQ ID NO:9)

A 1 L peptide reaction chamber will be charged 20.0 g 9-fluorenylmethyloxycarbonyl-(Fmoc)-Val-2-chlorotrityl resin. The resin will be conditioned in 200 mL (˜10 vol) of DCM with nitrogen agitation for about 15 minutes to swell the beads, and will then be drained.

Fmoc removal from the terminal amine will be carried out using 2×200 mL of a 20% solution of piperidine. The resin will then be washed with 200 mL of N-Methyl-2-pyrrolidone (NMP) to remove Fmoc by-products and residual piperidine, as determined by a negative chloranil test.

Meanwhile, Fmoc-4-methoxy-2,3,6-trimethylbenzenesulfonyl-(MTR)-Arg, will be activated for reaction at the carboxyl terminus. The Fmoc-protected amino acid (1.5 eq), the HOBT (1.5 eq), and the diisopropylethylamine (DIEA) (1.5 eq) will be dissolved in 150 mL (˜7.5 vol) of NMP at room temperature. The solution will be chilled to 0-5° C., then the HBTU (1.5 eq) will be added and stirred 5-15 minutes to dissolve. The solution of activated acid will be charged to the drained resin, and will be washed in with 50 mL of dichloromethane (DCM) (˜2.5 vol). The reaction will be agitated with nitrogen bubbling for 1 hr. Coupling completion will be monitored with the qualitative ninhydrin test. After the coupling reaction is deemed complete, the resin will be drained and washed with 3×200 mL (1 vol) of NMP.

The cycle will be repeated for subsequent amino acid residues of the peptide fragment using 1.5 equivalents each of Fmoc-protected amino acids Ser(tBu), Trp(Boc), Ser(tBu), Arg(MTR), Val, Val, Arg(MTR), Ser(tBu), and Arg(MTR). Following the final coupling reaction, the resin will be washed 4×200 mL (10 vol) of NMP, then with 4×200 mL (10 vol) of DCM. The resin will be dried with a nitrogen purge to give 42 g of resin-bound peptide.

The peptide will be cleaved from a 21 g quantity of the resin using 300 mL of 1% TFA in DCM for about 2 minutes, followed by 200 mL of 0.5% TFA in DCM. The cleavage fractions will be collected onto pyridine (1:1 volume ratio to TFA). The cleavage washes will be combined and concentrated under vacuum to a volume of ˜50 mL, then reconstituted with 110 mL of ethanol while the concentration will be continued to remove residual DCM to a final volume of ˜250 mL. Product will be precipitated with the addition of 200 mL of water. The slurry will be stirred at room temperature for 30 minutes. The solids will be collected by vacuum filtration and washed with ˜100 mL of water. The product will be air dried and purity will be assessed by HPLC.

The procedure can be repeated using various combinations of amino acids to chemically synthesize peptides as disclosed herein.

### Example 5: Synthesis in E. coli

A plasmid encoding an exemplary peptide of Formula D: Arg-Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg-Arg- Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg-Arg-Phe-Val-Arg-Arg-Val-Arg (SEQ ID NO:10) with an N terminal His6 tag (SEQ ID NO: 26) under control of a T7 promotor will be transformed into K12 Escherichia coli strain BL21(DE3). Colonies harboring the plasmid will be selected and used to produce a midlog phase culture for expression. Protein expression will be induced through addition of 0.1 mM isopropyl β-D-1-thiogalactopyranoside (IPTG).

Cells will be harvested and lysed using hen egg white lysozyme. The soluble fraction will be collected and will be subjected to Ni-NTA chromatography to purify the peptide. The peptide will then be further purified using gel filtration and ion exchange chromatography to produce a substantially pure peptide.

### Example 6: Formulation of a Composition

A peptide produced by the method described in Example 2 or 3 will be formulated as a composition for administration into an animal model. Briefly, a peptide will be diluted to an appropriate concentration in normal saline formulated with 0.01 μM EDTA. The formulation will be sterile filtered at 4° C. using a 0.2 micron filter. The formulation will be loaded into a syringe and stored at 4° C. for later use.

### Example 7: In Vitro Efficacy Against Bacteria

The following example illustrates a determination of in vitro efficacy of an exemplary peptide of Formula C: Arg-Arg-Thr-Tyr-Ser-Arg-Ser-Arg-Arg-Thr-Tyr-Ser-Arg-Ser-Arg-Arg-Thr-Tyr-Ser-Arg (SEQ ID NO:11).

Bacterial lysis assays will be conducted in a manner similar to that described previously (Lehrer, R. I., M. E. Selsted, D. Szklarek, and F. J. 1983. Infect. Immun. 42:10-4, 1983; Miller, M. A., R. F. Garry, J. M. Jaynes, and R. C. Montelaro, AIDS Res Hum Retroviruses 7:511-519, 1991). Bacterial suspensions will be grown to produce a midlog phase culture and will be washed with two cycles of centrifugation. The bacterial cells will be resuspended in 10 mM phosphate buffer and normalized to a concentration of 5×10⁵ cfu/mL. Bacteria will be incubated for 1 hour with two-fold dilutions of the peptide in 96-well plates using 10 mM phosphate buffer, pH 7.2, as a diluent. Ten-fold dilutions of bacteria will be produced; a 100 μL aliquot will be plated onto a surface of a tryptic soy agar plate and will be incubated overnight. Colonies of surviving bacteria will be quantified and compared to untreated controls to determine an amount of peptide-induced lysis. A minimal bactericidal concentration, MBC, defined as the peptide concentration at which 99.9% (three log) lysis is achieved, will be calculated.

### Example 8: In Vitro Efficacy Against Viruses

The following example illustrates a determination of in vitro efficacy of an exemplary peptide of Formula K: Lys-Val-Val-Ser-Ser-Ile-Ile-Glu-Ile-Ile-Ser-Ser-Val-Val-Lys-Val-Val-Ser-Ser-Ile-Ile-Glu-Ile-Ile-Ser-Ser-Val-Val (SEQ ID NO:12).

Human peripheral blood monocytes (PBMCs) will be obtained from healthy volunteers and maintained in culture at a concentration of 1×10⁵ viable cells per mL of medium. These cells will be stimulated by the addition of phytohemagglutinin (PHA). To this a standardized titer of purified HIV-1 (strain TIM) virions will be added to PBMCs to generate a p24 antigen signal of about 14,000 pg/mL five days post exposure to virus.

In order to test whether the peptide is able to suppress HIV-1 activity, the peptide at concentrations ranging between 0.1 and 1001.1M will be incubated with the standard virus titers for 30 min. Virions surviving peptide exposure will be isolated by ultracentrifugation at 100,000×g for 60 min. Viral pellets will be used to infect PHA stimulated PBMCs. Five days post infection, the level of p24 antigen will be determined and compared to a non-peptide treated control. The data will be expressed as the ratio of p24 antigen associated with peptide-treated vs. non-peptide treated HIV-1 infected cells to obtain a value referred to as percent suppression.

PK Studies

The following examples illustrate an administration of an exemplary peptide of SEQ ID NO:1 into various animal models.

### Example 9: PK in Macaques

Study Design

| Group | Number of | Test | Dose Level | Conc. | Dose Volume | | |
|---|---|---|---|---|---|---|---|
| Number | Animals | Article | (mg/kg) | (mg/mL) | (mL/kg) | Vehicle | Route |
| 1 | 3 | SEQ ID | 1.0 | 1.0 | 1 | SALINE | 30 MIN IV |
| 2 | 3 | NO: 1 | 3.0 | 3.0 | 1 | SALINE | INFUSION |
| 3 | 3 | | 10.0 | 10.0 | 1 | SALINE | |

Formulation Details

| | Compound Information | | |
|---|---|---|---|
| Test | (MW, FW, Correction | Test Article | Residual Formulation |
| Article | Factors, etc.) | Storage | Storage |
| SEQ ID | MW = 3399.14 FW = | −20° C. | −20° C. |
| NO: 1 | 4107.62 CF: 0.815 | | |

Formulation Instructions: NORMAL SALINE
Sampling and Storage: Residual dose formulation will be discarded following concentration analysis; results indicate administered formulation will be in the range of 100% ± 10% of the target dose concentration

Sample Collection

| Group Number | Serial Blood Collection Time |
|---|---|
| 1-3 | 0.0333, 0.25, 0.5, 1, 2, 4, 8, and 24 hours post |
| | dose (end of infusion) |
| Anticoagulant | K₂EDTA |
| Volume/Timepoint | ~1 mL |

Study Details

Animals will be obtained from the Test Facility's colony of adult male cynomolgous monkeys (Macaca fascicularis) of Chinese origin. Animals will be weighed prior to dosing.

Animals will be fasted a minimum of 2 hours prior to procedures requiring the administration of ketamine anesthesia. The diet will also be supplemented with other nutrients by feeding items such as raisins or fresh fruits that are presented to the animal as part of an environmental enrichment program. Offering items known to cause diarrhea will be avoided.

Intravenous administration will be via 30 minute infusion into a temporary percutaneous catheter placed into a saphenous vein. Prior to and following dose administration, the catheter will be flushed with 0.5 mL saline prior to removal. All dosing syringes will be weighed prior to and following dosing.

All animals will be observed at dosing and each scheduled collection. All abnormalities will be recorded.

Serial blood samples will be collected via femoral vein (cephalic or saphenous, as necessary). Approximately 1 mL blood samples will be collected into K₂EDTA tubes.

Blood samples will be collected into K₂EDTA tubes and stored on wet ice. Whole blood will be processed to plasma by centrifugation (2400-2700 rpm at 5° C.) within 30 minutes of collection. Plasma samples will be split into 2 equal aliquots and stored at −80° C. until analysis.

Administered doses will be determined gravimetrically.

Analysis

SEQ ID NO:1 will be purified from plasma samples from each macaque using a cation exchange-based solid-phase extraction process. Prior to extraction, a known concentration of a mass-adjusted internal standard (IS) will be added to assess recovery and allow for quantitation. Purified samples will be further subjected to high-performance liquid chromatography (HPLC) using C18 300 or 130 angstrom columns prior to MS/MS analysis. Multiple charge states will be observed by MS, so MS/MS will be employed to enable multiple reaction monitoring (MRM) analyses to determine the most MRM transitions with the highest signal-to-noise gain. Compound plasma concentrations will be determined by comparing to and compensating with co-purified IS peak intensity and validated calibrated standard curves.

Results

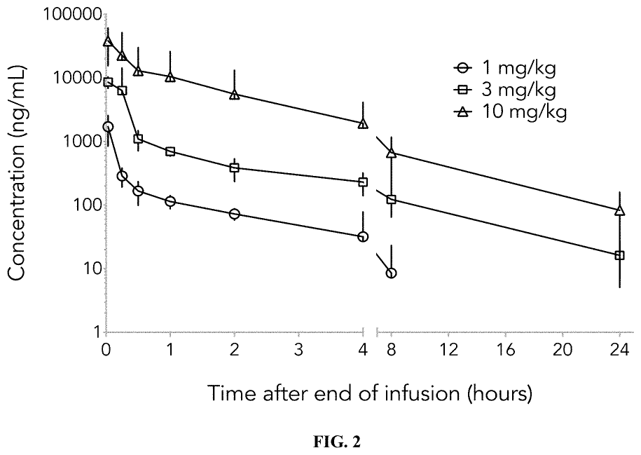

FIG. 2 depicts a plot of a mean serum concentration of the SEQ ID NO:1 peptide after administration to a cohort of macaques. Toxicokinetic parameters will be calculated using non-compartmental methods as implemented in the Phoenix® WinNonlin® ver. 6.3 comprehensive TK/PK analysis software program (Pharsight Corporation; Mountain View, Calif.) using an IV infusion model. Toxicokinetic analysis will be conducted using a 30-minute intravenous infusion time. Therefore, 30 minutes will be added to each sample time point postdose to obtain nominal time for TK analysis. Nominal times and dosage levels will be used for all calculations. Values below the lower limit of quantitation (BLQ) will be assigned a value of zero for the toxicokinetic calculations. Microsoft® Excel® 2013 will be used for receipt of bioanalytical data, minor formatting, including setting BLQ values to “0”, transfer into WinNonlin® for TK analysis, and calculation of the mean when an approximated value will be excluded. All plasma concentration data from all animals will be included in the analysis. Graphical presentations will be performed using Prism® for Macintosh ver. 7.0a (GraphPad, Inc.; La Jolla, Calif.).

PK profile parameters for IV infusion of a SEQ ID NO:1 formulation into macaques are recited below:

| | | | | | | | | | | | |
|---|---|---|---|---|---|---|---|---|---|---|---|
| | | | | Dose | | Dose | | | AUC_{inf} | | |
| | T_{1/2} | Tₘₐₓ | Cₘₐₓ | Normalized | AUCₗₐₛₜ | Normalized | AUC_{0-24 hr} | AUC_{inf} | Extrapolated | CL | Vₛₛ |
| Monkey | (hr) | (hr) | (ng/mL) | Cₘₐₓ | (hr*ng/mL) | AUCₗₐₛₜ | (hr*ng/mL) | (hr*ng/mL) | (%) | (mL/hr/kg) | (mL/kg) |

Group 1 IV Infusion Dose (1 mg/kg)

| | | | | | | | | | | | |
|---|---|---|---|---|---|---|---|---|---|---|---|
| 9912 | 1.7 * | 0.0333 | 1460 | 1460 | 740 | 740 | 821 | 938* | 21.0 | 1070* | 1530* |
| 9915 | 3.1 | 0.0333 | 2660 | 2660 | 1610 | 1610 | 1820 | 1730 | 6.5 | 579 | 1260 |
| 9946 | 0.94 | 0.0333 | 997 | 997 | 595 | 595 | 616 | 610 | 2.4 | 1640 | 1510 |
| Mean | 2 | 0.0333 | 1710 | 1710 | 983 | 983 | 1090 | 1170 | 10.0 | 1110 | 1380 |
| SD | NA | 0 | 858 | 858 | 551 | 551 | 643 | NA | 9.8 | NA | NA |

Group 2 IV Infusion Dose (3 mg/kg)

| | | | | | | | | | | | |
|---|---|---|---|---|---|---|---|---|---|---|---|
| 9912 | 4.4 | 0.0333 | 6830 | 2280 | 6650 | 2220 | 6650 | 6730 | 1.2 | 446 | 1580 |
| 9915 | 6.3 | 0.025 | 15500 | 5170 | 11000 | 3660 | 11000 | 11200 | 2.3 | 267 | 905 |
| 9946 | 5 | 0.0333 | 8690 | 2900 | 5120 | 1710 | 5120 | 5180 | 1.1 | 580 | 1370 |
| Mean | 5.2 | 0.11 | 10300 | 3450 | 7580 | 2530 | 7580 | 7710 | 1.5 | 431 | 1290 |
| SD | 0.95 | 0.13 | 4560 | 1520 | 3040 | 1010 | 3040 | 3150 | 0.7 | 157 | 344 |

Group 3 IV Infusion Dose (10 mg/kg)

| | | | | | | | | | | | |
|---|---|---|---|---|---|---|---|---|---|---|---|
| 9912 | 4.3 | 0.0333 | 26000 | 2600 | 22000 | 2200 | 22000 | 22200 | 1.0 | 450 | 1510 |
| 9915 | 4.9 | 0.0333 | 24300 | 2430 | 16400 | 1640 | 16400 | 16700 | 1.8 | 598 | 2010 |
| 9946 | 4.6 | 0.0333 | 64300 | 6430 | 112000 | 11200 | 112000 | 114000 | 0.99 | 88.1 | 256 |
| Mean | 4.6 | 0.0333 | 38200 | 3820 | 50300 | 5030 | 50300 | 50800 | 1.3 | 379 | 1260 |
| SD | 0.32 | 0 | 22600 | 2260 | 53900 | 5390 | 53900 | 54400 | 0.46 | 262 | 904 |

### Example 10: PK in Mice

Study Design

| Group | Number of | Test | Dose Level | Conc. | Dose Volume | | |
|---|---|---|---|---|---|---|---|
| Number | Animals | Article | (mg/kg) | (mg/mL) | (mL/kg) | Vehicle | Route* |
| 1 | 24 (3 per | SEQ ID | 3 | 0.6 | 5 | SALINE | IV |
| | timepoint) | NO: 1 | | | | | |
| 2 | 24 (3 per | | 15 | 3 | 5 | SALINE | IV |
| | timepoint) | | | | | | |

*Doses administered over ~30 seconds.

Formulation Details

| | Compound Information | | |
|---|---|---|---|
| Test | (MW, FW, Correction | Test Article | Residual Formulation |
| Article | Factors, etc.) | Storage | Storage |
| SEQ ID | MW = 3399.14: FW = | −20° C. | −20° C. |
| NO: 1 | 4107.62; CF: 0.815 | | |

Formulation Instructions: NORMAL SALINE
Sampling and Storage: Residual dose formulation will be stored at −80° C. until shipped to Sponsor.

Sample Collection

| Group Number | Terminal Blood Collection Time |
|---|---|
| 1-2 | 0.0333, 0.25, 0.5, 1, 2, 4, 8, and 24 hours post |
| | dose |
| | (3 animals/timepoint) |
| Anticoagulant | K₂EDTA |
| Volume/Timepoint | Maximum Obtainable |

Study Details

Male CD-1 mice will be received from an approved vendor and allowed to acclimate for a minimum of two days. Fasting will be not required.

All dosing syringes will be weighed prior to and following dosing. IV doses will be administered as a slow bolus (over ˜30 seconds) via direct venipuncture of a tail vein. All animals will be observed at dosing and each scheduled collection. All abnormalities will be recorded.

Terminal blood samples will be collected via cardiac puncture following inhalation anesthesia.

Sample Processing and Storage: Blood samples will be collected into K₂EDTA tubes and stored on wet ice. Whole blood will be processed to plasma by centrifugation (3500 rpm at 5° C.) within 30 minutes of collection. Plasma samples will be split into 2 equal aliquots and each transferred into 96 well plates (matrix tubes) and stored at −80° C. until analysis. Administered doses will be determined gravimetrically.

SEQ ID NO:1 will be purified from plasma samples from each mouse using a cation exchange-based solid-phase extraction process. Prior to extraction, a known concentration of a mass-adjusted internal standard (IS) will be added to assess recovery and allow for quantitation. Purified samples will be further subjected to high-performance liquid chromatography (HPLC) using C18 300 or 130 angstrom columns prior to MS/MS analysis. Multiple charge states will be observed by MS, so MS/MS will be employed to enable multiple reaction monitoring (MRM) analyses to determine the most MRM transitions with the highest signal-to-noise gain. Compound plasma concentrations will be determined by comparing to and compensating with co-purified IS peak intensity and validated calibrated standard curves.

Results

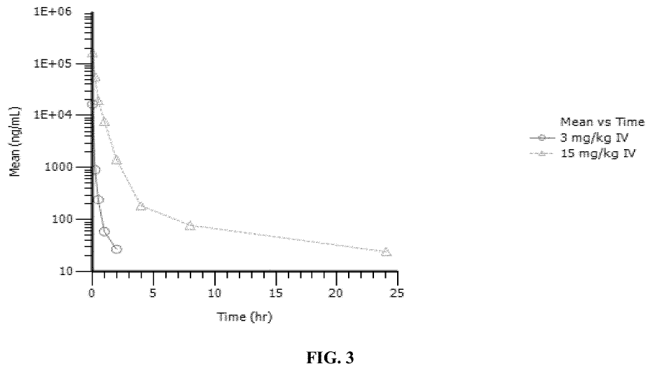

FIG. 3 depicts a plot of a mean serum concentration of SEQ ID NO:1 after administration to a cohort of mice. Pharmacokinetic parameters will be calculated using non-compartmental methods as implemented in the Phoenix® WinNonlin® ver. 6.3 comprehensive TK/PK analysis software program (Pharsight Corporation; Mountain View, Calif.) using an IV infusion model. Pharmacokinetic analysis will be conducted using a 30-minute intravenous infusion time. Therefore, 30 minutes will be added to each sample time point postdose to obtain nominal time for TK analysis. Nominal times and dosage levels will be used for all calculations. Values below the lower limit of quantitation (BLQ) will be assigned a value of zero for the pharmacokinetic calculations. Microsoft® Excel® 2013 will be used for receipt of bioanalytical data, minor formatting, including setting BLQ values to “0”, transfer into WinNonlin® for PK analysis, and calculation of the mean when an approximated value will be excluded. All plasma concentration data from all animals will be included in the analysis. Graphical presentations will be performed using Microsoft® Excel® 2013.

Exemplary PK profile parameters for IV infusion of a SEQ ID NO:1 formulation into mice are recited below:

| | | | | | | | | | | | | |
|---|---|---|---|---|---|---|---|---|---|---|---|---|
| | | | | | | | | Extrap- | | | | |
| | T_{1/2} | Tₘₐₓ | Cₘₐₓ | C₀ | AUCₗₐₛₜ | AUC_{inf} | AUCI_{inf}_{_{—}}_{D} | olated | Vz | CL | MRTₗₐₛₜ | Vₛₛ |
| Mouse | (hr) | (hr) | (ng/mL) | (ng/mL) | (hr*ng/mL) | (hr*ng/mL) | (hr*kg*ng/mL/ng) | (%) | (mL/kg) | (mL/hr/kg) | (hr) | (mL/kg) |

Group 1 IV Dose (3 mg/kg)

| | | | | | | | | | | | | |
|---|---|---|---|---|---|---|---|---|---|---|---|---|
| 1 | ~8 | 0.0333 | 16633 | 26008 | 2874 | <3500 | <3500 | ~0.5 | ~3000 | ~300 | ~0.5 | ~200 |

Group 2 IV Dose (15 mg/kg)

| | | | | | | | | | | | | |
|---|---|---|---|---|---|---|---|---|---|---|---|---|
| 2 | 7.45 | 0.0333 | 162667 | 191509 | 53578 | 53842 | 3589 | 0.49 | 2996 | 279 | 0.579 | 208 |

### Example 11: PK in Rats

Study Design

| Group | Number of | Test | Dose Level | Conc. | Dose Volume | | |
|---|---|---|---|---|---|---|---|
| Number | Animals | Article | (mg/kg) | (mg/mL) | (mL/kg) | Vehicle | Route |
| 1 | 5 | SEQ ID | 1 | 0.2 | 5 | SALINE | 30 MIN IV |
| 2 | 5 | NO: 1 | 5 | 1 | 5 | SALINE | INFUSION |
| 3 | 5 | | 15 | 3 | 5 | SALINE | |
| 4 | 2* | SEQ ID | 15* | 3 | 5 | SALINE | 60 MIN IV |
| | | NO: 1 | | | | | INFUSION |

*If not tolerated, dose level can be reduced to 10 mg/kg (2 mg/ml) for a 30 minute infusion in 5 additional rats. If tolerated, 3 additional rats can be dosed at this level.

Formulation Details

| | Compound Information | | |
|---|---|---|---|
| Test | (MW, FW, Correction | Test Article | Residual Formulation |
| Article | Factors, etc.) | Storage | Storage |
| SEQ ID | MW = 3399.14 FW = | −20° C. | −20° C. |
| NO: 1 | 4107.62 CF: 0.815 | | |

Formulation Instructions: NORMAL SALINE
Sampling and Storage: Residual dose formulation will be stored at −80° C. until final disposition.

Sample Collection

| Group Number | Serial Blood Collection Time |
|---|---|
| 1-3 | 0.0333, 0.25, 0.5, 1, 2, 4, 8, and 24 hours post |
| | dose |
| Anticoagulant | K₂EDTA |
| Volume/Timepoint | ~250 μL |

Study Details

Male Sprague-Dawley Rats will be received from an approved vendor with a single jugular vein cannula (JVC) and allowed to acclimate to the Test Facility for at least 2 days prior to study start. Animals will be weighed prior to dosing. No fasting will be required.

Intravenous administration will be via 30 minute infusion into a JVC. Following dosing, the catheter will be flushed with ˜0.5 mL saline and tied off to prevent re-access.

All animals will be observed at dosing and each scheduled collection. All abnormalities will be recorded.

Serial blood samples will be collected via jugular vein cannula (JVC) or by another approved method if patency is lost. The final blood samples will be obtained via direct cardiac puncture following inhalation anesthesia.

Blood samples will be collected into K₂EDTA tubes and stored on wet ice. Whole blood will be processed to plasma by centrifugation (3500 rpm at 5° C.) within 30 minutes of collection. Plasma samples will be transferred into 96 well plates (matrix tubes) and stored at −80° C. until analysis. Administered doses will be determined gravimetrically.

Analysis

SEQ ID NO:1 will be purified from plasma samples from each rat using a cation exchange-based solid-phase extraction process. Prior to extraction, a known concentration of a mass-adjusted internal standard (IS) will be added to assess recovery and allow for quantitation. Purified samples will be further subjected to high-performance liquid chromatography (HPLC) using C18 300 or 130 angstrom columns prior to MS/MS analysis. Multiple charge states will be observed by MS, so MS/MS will be employed to enable multiple reaction monitoring (MRM) analyses to determine the most MRM transitions with the highest signal-to-noise gain. Compound plasma concentrations will be determined by comparing to and compensating with co-purified IS peak intensity and validated calibrated standard curves.

Results

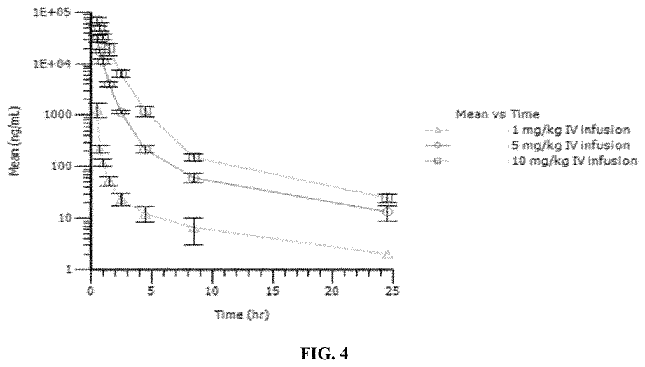

FIG. 4 depicts a plot of a mean serum concentration of SEQ ID NO:1 after administration to a cohort of rats. Pharmacokinetic parameters will be calculated using non-compartmental methods as implemented in the Phoenix® WinNonlin® ver. 6.3 comprehensive TK/PK analysis software program (Pharsight Corporation; Mountain View, Calif.) using an IV infusion model. Pharmacokinetic analysis will be conducted using a 30-minute intravenous infusion time. Therefore, 30 minutes will be added to each sample time point postdose to obtain nominal time for TK analysis. Nominal times and dosage levels will be used for all calculations. Values below the lower limit of quantitation (BLQ) will be assigned a value of zero for the pharmacokinetic calculations. Microsoft® Excel® 2013 will be used for receipt of bioanalytical data, minor formatting, including setting BLQ values to “0”, transfer into WinNonlin® for PK analysis, and calculation of the mean when an approximated value will be excluded. All plasma concentration data from all animals will be included in the analysis. Graphical presentations will be performed using Microsoft® Excel® 2013.

Exemplary PK profile parameters for IV infusion of a SEQ ID NO:1 formulation into rats are recited below:

| | | | | | | | | | | | |
|---|---|---|---|---|---|---|---|---|---|---|---|
| | | | | | | | | | | MRT | |
| | T _{1/2} | Tₘₐₓ | Cₘₐₓ | AUCₗₐₛₜ | AUC_{inf} | AUCI_{inf}_{_{—}}_{D} | Extrapolated | Vz | CL | ₗₐₛₜ | Vₛₛ |
| Rat | (hr) | (hr) | (ng/mL) | (hr*ng/mL) | (hr*ng/mL) | (hr*kg*ng/mL/ng) | (%) | (mL/kg) | (mL/hr/kg) | (hr) | (mL/kg) |

Group 1 IV Infusion Dose (1 mg/kg)

| | | | | | | | | | | | |
|---|---|---|---|---|---|---|---|---|---|---|---|
| 1 | 13.8 | 0.5333 | 1547 | 947 | 1051 | 1051 | 9.88 | 18997 | 951 | 2.52 | 6310 |
| 2 | 5.92 | 0.5333 | 795 | 595 | 606 | 606 | 1.86 | 14090 | 1650 | 2.21 | 4591 |
| 3 | 2.31 | 0.5333 | 1605 | 810 | 820 | 820 | 1.28 | 4061 | 1219 | 0.655 | 970 |
| 4 | 6.06 | 0.5333 | 897 | 710 | 724 | 724 | 1.91 | 12077 | 1381 | 2.39 | 4114 |
| 5 | 2.65 | 0.5333 | 1512 | 724 | 735 | 735 | 1.48 | 5195 | 1360 | 0.581 | 1021 |
| Mean | 6.16 | 0.5333 | 1271 | 757 | 787 | 787 | 3.28 | 10884 | 1312 | 1.67 | 3401 |
| SD | 4.64 | 0.00 | 391 | 131 | 166 | 166 | 3.70 | 6254 | 255 | 0.969 | 2343 |

Group 2 IV Infusion Dose (5 mg/kg)

| | | | | | | | | | | | |
|---|---|---|---|---|---|---|---|---|---|---|---|
| 6 | 5.76 | 0.5333 | 27625 | 25472 | 25629 | 5126 | 0.612 | 1622 | 195 | 1.18 | 268 |
| 7 | 5.98 | 0.5333 | 28112 | 24511 | 24665 | 4933 | 0.624 | 1748 | 203 | 1.12 | 267 |
| 8 | 5.14 | 0.5333 | 27948 | 25397 | 25479 | 5096 | 0.320 | 1455 | 196 | 1.02 | 219 |
| 9 | 4.91 | 0.5333 | 39516 | 29770 | 29831 | 5966 | 0.204 | 1186 | 168 | 0.877 | 157 |
| 10 | 5.36 | 0.5333 | 28844 | 23267 | 23351 | 4670 | 0.358 | 1656 | 214 | 0.979 | 233 |
| Mean | 5.43 | 0.5333 | 30409 | 25683 | 25791 | 5158 | 0.424 | 1533 | 195 | 1.03 | 229 |
| SD | 0.440 | 0.00 | 5110 | 2452 | 2432 | 486 | 0.186 | 221 | 17.2 | 0.119 | 45.2 |

Group 4 IV Infusion Dose (10 mg/kg)

| | | | | | | | | | | | |
|---|---|---|---|---|---|---|---|---|---|---|---|
| 21 | 4.22 | 0.5333 | 59822 | 70290 | 70458 | 7046 | 0.238 | 864 | 142 | 1.24 | 186 |
| 22 | 4.24 | 0.5333 | 53583 | 64076 | 64204 | 6420 | 0.199 | 954 | 156 | 1.16 | 190 |
| 23 | 3.96 | 0.5333 | 84201 | 85892 | 86014 | 8601 | 0.142 | 665 | 116 | 1.12 | 135 |
| 24 | 4.14 | 0.5333 | 64187 | 87269 | 87454 | 8745 | 0.211 | 683 | 114 | 1.27 | 152 |
| 25 | 3.80 | 0.5333 | 74164 | 94170 | 94302 | 9430 | 0.140 | 581 | 106 | 1.12 | 123 |
| Mean | 4.07 | 0.5333 | 67192 | 80339 | 80486 | 8049 | 0.186 | 749 | 127 | 1.18 | 157 |
| SD | 0.188 | 0.00 | 12109 | 12606 | 12606 | 1261 | 0.0435 | 154 | 21.0 | 0.0711 | 30.3 |

### Example 12: PK in Dogs

Study Design

| Group | Number of | Test | Dose Level | Conc. | Dose Volume | | |
|---|---|---|---|---|---|---|---|
| Number | Animals | Article | (mg/kg) | (mg/mL) | (mL/kg) | Vehicle | Route |
| 1 | 1 | SEQ ID | 0.1 | 0.1 | 1 | SALINE | 30 MIN IV |
| 2 | 1 | NO: 1 | 0.5 | 0.5 | 1 | SALINE | INFUSION |
| 3 | 3 | | 1 | 1 | 1 | SALINE | |

Formulation Details

| | Compound Information | | |
|---|---|---|---|
| Test | (MW, FW, Correction | Test Article | Residual Formulation |
| Article | Factors, etc.) | Storage | Storage |
| SEQ ID | MW = 3399.14 FW = | −20° C. | −20° C. |
| NO: 1 | 4107.62 CF: 0.815 | | |

Formulation Instructions: NORMAL SALINE
Sampling and Storage: Residual dose formulation will be stored at −80° C. until shipped to Sponsor.

Sample Collection

| Group Number | Serial Blood Collection Time |
|---|---|
| 1-3 | 0.0333, 0.25, 0.5, 1, 2, 4, 8, and 24 hours post |
| | dose (end of infusion) |
| Anticoagulant | K₂EDTA |
| Volume/Timepoint | ~1 mL |

Study Details

Animals will be obtained from the Test Facility's colony of protein-naïve animals. Animals will be weighed prior to dosing.

Intravenous administration will be via 30 minute infusion into a temporary percutaneous catheter placed into a saphenous vein. Following dose administration, the catheter will be flushed with 3 mL saline prior to removal. All dosing syringes will be weighed prior to and following dosing.

All animals will be observed at dosing and each scheduled collection. All abnormalities will be recorded.

Serial blood samples will be collected via cephalic vein, or other peripheral vein (jugular or saphenous), as necessary. Approximately 1 mL blood samples will be collected into K₂EDTA tubes.

Blood samples will be collected into K₂EDTA tubes and stored on wet ice. Whole blood will be processed to plasma by centrifugation (3500 rpm at 5° C.) within 30 minutes of collection. Plasma samples will be split into 2 equal aliquots and each transferred into 96 well plates (matrix tubes) and stored at −80° C. until analysis. The second set will be retained at the dosing facility until final disposition.

Administered doses will be determined gravimetrically.

Analysis

SEQ ID NO:1 will be purified from plasma samples from each dog using a cation exchange-based solid-phase extraction process. Prior to extraction, a known concentration of a mass-adjusted internal standard (IS) will be added to assess recovery and allow for quantitation. Purified samples will further subjected to high-performance liquid chromatography (HPLC) using C18 300 or 130 angstrom columns prior to MS/MS analysis. Multiple charge states will be observed by MS, MS/MS will employed to enable multiple reaction monitoring (MRM) analyses to determine the most MRM transitions with the highest signal-to-noise gain. Compound plasma concentrations will be determined by comparing to and compensating with co-purified IS peak intensity and validated calibrated standard curves.

### Example 13: PK in Macaques

The following example illustrates an administration of an peptide of Formula C (Lys-Lys-Thr-His-Thr-Lys-Thr-Lys-Lys-Thr-His-Thr-Lys-Thr-Lys-Lys-Thr-His-Thr-Lys; SEQ ID NO:13) into maques.

Study Design

| Group | Number of | Test | Dose Level | Conc. | Dose Volume | | |
|---|---|---|---|---|---|---|---|
| Number | Animals | Article | (mg/kg) | (mg/mL) | (mL/kg) | Vehicle | Route |
| 1 | 3 | SEQ ID | 1.0 | 1.0 | 1 | SALINE + | 30 MIN IV |
| | | NO: 13 | | | | 0.01% GLYCEROL | INFUSION |

Formulation Details

| | Compound Information | | |
|---|---|---|---|
| Test | (MW, FW, Correction | Test Article | Residual Formulation |
| Article | Factors, etc.) | Storage | Storage |
| SEQ ID | MW = 7429.27 | −20° C. | −20° C. |
| NO: 13 | | | |

Formulation Instructions: NORMAL SALINE + 0.01% GLYCEROL
Sampling and Storage: Residual dose formulation will be discarded following concentration analysis; results indicate administered formulation will be in the range of 100% ± 10% of the target dose concentration

Sample Collection

| Group Number | Serial Blood Collection Time |
|---|---|
| 1-3 | 0.0333, 0.25, 0.5, 1, 2, 4, 8, and 24 hours post |
| | dose (end of infusion) |
| Anticoagulant | K₂EDTA |
| Volume/Timepoint | ~1 mL |

Study Details

Animals will be obtained from the Test Facility's colony of adult male cynomolgous monkeys (Macaca fascicularis) of Chinese origin. Animals will be weighed prior to dosing.

Animals will be fasted a minimum of 2 hours prior to procedures requiring the administration of ketamine anesthesia. The diet will also be supplemented with other nutrients by feeding items such as raisins or fresh fruits that are presented to the animal as part of an environmental enrichment program. Offering items known to cause diarrhea will be avoided.

Intravenous administration will be via 30 minute infusion into a temporary percutaneous catheter placed into a saphenous vein. Prior to and following dose administration, the catheter will be flushed with 0.5 mL saline prior to removal. All dosing syringes will be weighed prior to and following dosing.

All animals will be observed at dosing and each scheduled collection. All abnormalities will be recorded.

Serial blood samples will be collected via femoral vein (cephalic or saphenous, as necessary). Approximately 1 mL blood samples will be collected into K₂EDTA tubes.

Blood samples will be collected into K₂EDTA tubes and stored on wet ice. Whole blood will be processed to plasma by centrifugation (2400-2700 rpm at 5° C.) within 30 minutes of collection. Plasma samples will be split into 2 equal aliquots and stored at −80° C. until analysis.

Administered doses will be determined gravimetrically.

Analysis

The peptide will be purified from plasma samples from each macaque using a cation exchange-based solid-phase extraction process. Prior to extraction, a known concentration of a mass-adjusted internal standard (IS) will be added to assess recovery and allow for quantitation. Purified samples will be further subjected to high-performance liquid chromatography (HPLC) using C18 300 or 130 angstrom columns prior to MS/MS analysis. Multiple charge states will be observed by MS, so MS/MS will be employed to enable multiple reaction monitoring (MRM) analyses to determine the most MRM transitions with the highest signal-to-noise gain. Compound plasma concentrations will be determined by comparing to and compensating with co-purified IS peak intensity and validated calibrated standard curves.

Results

Toxicokinetic parameters will be calculated using non-compartmental methods as implemented in the Phoenix® WinNonlin® ver. 6.3 comprehensive TK/PK analysis software program (Pharsight Corporation; Mountain View, Calif.) using an IV infusion model. Toxicokinetic analysis will be conducted using a 30-minute intravenous infusion time. Therefore, 30 minutes will be added to each sample time point postdose to obtain nominal time for TK analysis. Nominal times and dosage levels will be used for all calculations. Values below the lower limit of quantitation (BLQ) will be assigned a value of zero for the toxicokinetic calculations. Microsoft® Excel® 2013 will be used for receipt of bioanalytical data, minor formatting, including setting BLQ values to “0”, transfer into WinNonlin® for TK analysis, and calculation of the mean when an approximated value will be excluded. All plasma concentration data from all animals will be included in the analysis. Graphical presentations will be performed using Prism® for Macintosh ver. 7.0a (GraphPad, Inc.; La Jolla, Calif.).

PK profile parameters for IV infusion of the peptide formulation into macaques are recited below:

| | T_{1/2} | Tₘₐₓ | Cₘₐₓ | Dose Normalized | AUCₗₐₛₜ | Dose Normalized | AUC_{0-24 hr} | AUC_{inf} |
|---|---|---|---|---|---|---|---|---|
| Monkey | (hr) | (hr) | (ng/mL) | Cₘₐₓ | (hr*ng/mL) | AUCₗₐₛₜ | (hr*ng/mL) | (hr*ng/mL) |
| 4000 | 6.1 | 0.05 | 5000 | 5000 | 1200 | 1200 | 1400 | 1480 |
| 4001 | 8.1 | 0.05 | 7000 | 7000 | 4000 | 4000 | 4200 | 4238 |
| 4002 | 7.6 | 0.05 | 6000 | 6000 | 3200 | 3200 | 3400 | 3370 |

### Example 14: Pretreatment with a Biofilm Disrupter

P. aeruginosa or S. aureus cells will be grown in vinyl microtiter plates in Mueller-Hinton broth for 24 h at 37° C. to allow a mature biofilm to form. After about 24 hrs, a solution of approximately 0.1 mM benzalkonium chloride will be added to the biofilm containing wells and incubated for 1 hour. To this solution will be added an exemplary peptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13, diluted 2-fold in MHB. The resulting mixture will be incubated for about 1 hour. The well-associated biomass will be stained with crystal violet and quantified by measuring absorbance at 550 nm following solubilization in 30% acetic acid. Biofilm disruption will be quantitated by the absorbance at 550 nm as a function of the concentration of peptide.

### Example 15: Administration of Compositions with a Biofilm Disrupter

P. aeruginosa or S. aureus cells will be grown in vinyl microtiter plates in Mueller-Hinton broth for 24 h at 37° C. to allow a mature biofilm to form. After about 24 hrs, a composition containing 0.1% w/w cysteamine, an exemplary peptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13, and polysorbate 80 will be diluted 2-fold in MHB. The resulting mixture will be incubated for about 1 hour. The well-associated biomass will be stained with crystal violet and quantified by measuring absorbance at 550 nm following solubilization in 30% acetic acid. Biofilm disruption will be quantitated by the absorbance at 550 nm as a function of the concentration of peptide.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1.-43. (canceled)

44. A pharmaceutical formulation comprising:
a. a peptide or salt thereof comprising from about 70% to about 100% homology to a polypeptide of sequence:
(SEQ ID NO: 15)
Arg-Val-Val-Arg-Val-Val-Arg-Arg-Val-Val-Arg-Arg;
(SEQ ID NO: 16)
Arg-Val-Val-Arg-Val-Val-Arg-Arg-Trp-Val-Arg-Arg;
(SEQ ID NO: 17)
Arg-Trp-Trp-Arg-Trp-Trp-Arg-Arg-Trp-Trp-Arg-Arg;
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
or
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val;
b. at least one of: an excipient, a diluent, or a carrier;
wherein the pharmaceutical formulation is in unit dose form, wherein the peptide does not comprise 3 or more contiguous arginine or lysine residues; wherein the peptide is not a cyclic peptide; and wherein at least one of the following applies:
(i) the peptide, a metabolite thereof, or salt thereof exhibits antimicrobial activity against a bacteria with a minimum inhibitory concentration ranging from about 0.1 μg/mL to about 100 μg/mL in vitro;
(ii) the peptide, a metabolite thereof, or salt thereof exhibits antifungal activity against a fungus with a minimum inhibitory concentration ranging from about 0.1 μg/mL to about 100 μg/mL in vitro;
(iii) the peptide, a metabolite thereof, or salt thereof exhibits antiviral activity against a virus with a minimum inhibitory concentration ranging from about 0.1 μg/mL to about 100 μg/mL in vitro; or
(iv) the peptide, a metabolite thereof, or salt thereof exhibits antitumor activity against a tumor cell with an LD₅₀ of from about 0.01 μM to about 100 μM in vitro.

45.-53. (canceled)

54. The pharmaceutical formulation of claim 44, wherein when the pharmaceutical formulation is administered to a subject, the peptide or salt thereof has a Tₘₐₓ of from about 1 minute to about 1 hour, a Cₘₐₓ of at least about 100 ng/mL, an AUC_{0>24} hour of from about 0.1 μg·hr/L to about 1,000 μg·hr/L, or a combination thereof.

55. The pharmaceutical formulation of claim 44, wherein when the pharmaceutical formulation is administered to a subject, the peptide or salt thereof is substantially localized in a liver, a spleen, or a kidney of the subject.

56. The pharmaceutical formulation of claim 44, wherein when the pharmaceutical formulation is administered to a subject, the peptide or salt thereof has a half-life that is from about 2 hours to about 24 hours.

57.-152. (canceled)

153. The pharmaceutical formulation of claim 44, wherein the peptide, a metabolite thereof, or salt thereof exhibits antimicrobial activity against a Streptococcus agalactiae bacteria strain at a minimum inhibitory concentration that is at least two-fold lower than a minimum inhibitory concentration for an antimicrobial activity against a Streptococcus pneumoniae bacteria strain in vitro.

154. The pharmaceutical formulation of claim 44, wherein the peptide, a metabolite thereof, or salt thereof exhibits antimicrobial activity against a multidrug-resistant Acinetobacter baumannii bacteria strain at a minimum inhibitory concentration of from about 0.5 μg/mL to about 32 μg/mL in vitro.

155. The pharmaceutical formulation of claim 44, wherein the peptide, a metabolite thereof, or salt thereof exhibits antimicrobial activity against a vancomycin-resistant Enterococcus faecium bacteria strain at a minimum inhibitory concentration that is at least two-fold lower than a minimum inhibitory concentration for an antimicrobial activity against a vancomycin-sensitive Enterococcus faecium bacteria strain in vitro.

156. The pharmaceutical formulation of claim 44, wherein the peptide or salt thereof comprises at least about 95% homology to the polypeptide of sequence Arg Arg Trp Val Arg Arg Val Arg Arg Val Trp Arg Arg Val Val Arg Val Val Arg Arg Trp Val Arg Arg (SEQ ID NO: 1).

157. The pharmaceutical formulation of claim 44, wherein the peptide or salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1).

158. The pharmaceutical formulation of claim 44, comprising the excipient, wherein the excipient is a chelator.

159. The pharmaceutical formulation of claim 158, wherein the chelator is a fungicidal chelator.

160. The pharmaceutical formulation of claim 44, comprising the diluent, wherein the diluent is an aqueous acid.

161. The pharmaceutical formulation of claim 44, comprising the diluent, wherein the pharmaceutical formulation is formulated to physiological pH using the diluent.

162. The pharmaceutical formulation of claim 44, comprising the diluent, wherein the pharmaceutical formulation is formulated to a pH that is not physiological pH using the diluent.

163. The pharmaceutical formulation of claim 44, further comprising cysteamine.

164. The pharmaceutical formulation of claim 44, further comprising a surfactant.

165. The pharmaceutical formulation of claim 164, wherein the surfactant is selected from the group consisting of a polyoxyethylene sorbitan fatty acid ester, sodium lauryl sulphate, sodium stearyl fumarate, a polyoxyethylene alkyl ether, a sorbitan fatty acid ester, polyethylene glycols, a polyoxyethylene castor oil derivative, docusate sodium, a quaternary ammonium compound, a sugar ester of a fatty acid, a glyceride of a fatty acid, and any combination thereof.

166. The pharmaceutical formulation of claim 44, further comprising a small molecule selected from the group consisting of imidazole, indole, nitric oxide, a triazole, a phenol, a sulfide, a polysaccharide, a furanone, a bromopyrrole, a salt of any of these, and any combination thereof.

167. The pharmaceutical formulation of claim 44, that is in the form of a tablet, a liquid, a syrup, an oral formulation, an intravenous formulation, an intranasal formulation, an ocular formulation, an otic formulation, a subcutaneous formulation, an inhalable respiratory formulation, or a suppository.

168. The pharmaceutical formulation of claim 44, wherein at least about 80% by weight of the peptide or salt thereof is present at the end of a 2 year period, as determined by:
(a) loading a sample of the peptide or salt thereof on a high performance liquid chromatography (HPLC) equipped with a size exclusion column that is at least about 6 inches in length and comprises a silica gel; and
(b) performing mass spectroscopy on at least one sample eluted from the size exclusion column;
wherein said pharmaceutical formulation is stored in a closed container at 25° C. at 50% atmospheric relative humidity.

169. The pharmaceutical formulation of claim 44, further comprising an additional antibiotic.

170. The pharmaceutical formulation of claim 169, wherein the additional antibiotic is selected from the group consisting of Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof.

171. A kit comprising the pharmaceutical formulation of claim 44 in a container.

172. The kit of claim 171, further comprising a syringe.

173. A method of making a kit, comprising combining the pharmaceutical formulation of claim 44 with a container.
